SEQUENCE LISTING (16 sequences)

SEQ ID NO: 1 - protein, 883 aa, unidentified
  source 1..883: note = Bacteriophage T7
MNTINIAKNDFSDIELAAIPFNTLADHYGERLAREQLALEHESYEMGEARFRKMFERQLKAGEVADNAAAKPLITTLLPKMIARINDWFEEVKAKRGKRPTAFQFLQEIKPEAVAYITIKTTLACLTSADNTTVQAVASAIGRAIEDEARFGRIRDLEAKHFKKNVEEQLNKRVGHVYKKAFMQVVEADMLSKGLLGGEAWSSWHKEDSIHVGVRCIEMLIESTGMVSLHRQNAGVVGQDSETIELAPEYAEAIATRAGALAGISPMFQPCVVPPKPWTGITGGGYWANGRRPLALVRTHSKKALMRYEDVYMPEVYKAINIAQNTAWKINKKVLAVANVITKWKHCPVEDIPAIEREELPMKPEDIDMNPEALTAWKRAAAAVYRKDKARKSRRISLEFMLEQANKFANHKAIWFPYNMDWRGRVYAVSMFNPQGNDMTKGLLTLAKGKPIGKEGYYWLKIHGANCAGVDKVPFPERIKFIEENHENIMACAKSPLENTWWAEQDSPFCFLAFCFEYAGVQHHGLSYNCSLPLAFDGSCSGIQHFSAMLRDEVGGRAVNLLPSETVQDIYGIVAKKVNEILQADAINGTDNEVVTVTDENTGEISEKVKLGTKALAGQWLAYGVTRSVTKRSVMTLAYGSKEFGFRQQVLEDTIQPAIDSGKGLMFTQPNQAAGYMAKLIWESVSVTVVAAVEAMNWLKSAAKLLAAEVKDKKTGEILRKRCAVHWVTPDGFPVWQEYKKPIQTRLNLMFLGQFRLQPTINTNKDSEIDAHKQESGIAPNFVHSQDGSHLRKTVVWAHEKYGIESFALIHDSFGTIPADAANLFKAVRETMVDTYESCDVLADFYDQFADQLHESQLDKMPALPAKGNLNLRDILESDFAFA

SEQ ID NO: 2 - protein, 883 aa, synthetic construct
  VARIANT 350: note = X may be W, Y, or F
  VARIANT 351: note = X may be A, G, I, L, P, or V
  VARIANT 387: note = X may be N, C, Q, M, S, or T
  VARIANT 437: note = X may be W, Y, or F
MNTINIAKNDFSDIELAAIPFNTLADHYGERLAREQLALEHESYEMGEARFRKMFERQLKAGEVADNAAAKPLITTLLPKMIARINDWFEEVKAKRGKRPTAFQFLQEIKPEAVAYITIKTTLACLTSADNTTVQAVASAIGRAIEDEARFGRIRDLEAKHFKKNVEEQLNKRVGHVYKKAFMQVVEADMLSKGLLGGEAWSSWHKEDSIHVGVRCIEMLIESTGMVSLHRQNAGVVGQDSETIELAPEYAEAIATRAGALAGISPMFQPCVVPPKPWTGITGGGYWANGRRPLALVRTHSKKALMRYEDVYMPEVYKAINIAQNTAWKINKKVLAVANVITKWKHCPVXXIPAIEREELPMKPEDIDMNPEALTAWKRAAAAVYRXDKARKSRRISLEFMLEQANKFANHKAIWFPYNMDWRGRVYAVSMFNPQGXDMTKGLLTLAKGKPIGKEGYYWLKIHGANCAGVDKVPFPERIKFIEENHENIMACAKSPLENTWWAEQDSPFCFLAFCFEYAGVQHHGLSYNCSLPLAFDGSCSGIQHFSAMLRDEVGGRAVNLLPSETVQDIYGIVAKKVNEILQADAINGTDNEVVTVTDENTGEISEKVKLGTKALAGQWLAYGVTRSVTKRSVMTLAYGSKEFGFRQQVLEDTIQPAIDSGKGLMFTQPNQAAGYMAKLIWESVSVTVVAAVEAMNWLKSAAKLLAAEVKDKKTGEILRKRCAVHWVTPDGFPVWQEYKKPIQTRLNLMFLGQFRLQPTINTNKDSEIDAHKQESGIAPNFVHSQDGSHLRKTVVWAHEKYGIESFALIHDSFGTIPADAANLFKAVRETMVDTYESCDVLADFYDQFADQLHESQLDKMPALPAKGNLNLRDILESDFAFA

SEQ ID NO: 3 - protein, 883 aa, synthetic construct
  VARIANT 350: note = X may be W, Y, or F
  VARIANT 351: note = X may be A, G, I, L, P, or V
  VARIANT 437: note = X may be W, Y, or F
MNTINIAKNDFSDIELAAIPFNTLADHYGERLAREQLALEHESYEMGEARFRKMFERQLKAGEVADNAAAKPLITTLLPKMIARINDWFEEVKAKRGKRPTAFQFLQEIKPEAVAYITIKTTLACLTSADNTTVQAVASAIGRAIEDEARFGRIRDLEAKHFKKNVEEQLNKRVGHVYKKAFMQVVEADMLSKGLLGGEAWSSWHKEDSIHVGVRCIEMLIESTGMVSLHRQNAGVVGQDSETIELAPEYAEAIATRAGALAGISPMFQPCVVPPKPWTGITGGGYWANGRRPLALVRTHSKKALMRYEDVYMPEVYKAINIAQNTAWKINKKVLAVANVITKWKHCPVXXIPAIEREELPMKPEDIDMNPEALTAWKRAAAAVYRKDKARKSRRISLEFMLEQANKFANHKAIWFPYNMDWRGRVYAVSMFNPQGXDMTKGLLTLAKGKPIGKEGYYWLKIHGANCAGVDKVPFPERIKFIEENHENIMACAKSPLENTWWAEQDSPFCFLAFCFEYAGVQHHGLSYNCSLPLAFDGSCSGIQHFSAMLRDEVGGRAVNLLPSETVQDIYGIVAKKVNEILQADAINGTDNEVVTVTDENTGEISEKVKLGTKALAGQWLAYGVTRSVTKRSVMTLAYGSKEFGFRQQVLEDTIQPAIDSGKGLMFTQPNQAAGYMAKLIWESVSVTVVAAVEAMNWLKSAAKLLAAEVKDKKTGEILRKRCAVHWVTPDGFPVWQEYKKPIQTRLNLMFLGQFRLQPTINTNKDSEIDAHKQESGIAPNFVHSQDGSHLRKTVVWAHEKYGIESFALIHDSFGTIPADAANLFKAVRETMVDTYESCDVLADFYDQFADQLHESQLDKMPALPAKGNLNLRDILESDFAFA

SEQ ID NO: 4 - protein, 883 aa, synthetic construct
  VARIANT 350: note = X may be W, Y, or F
  VARIANT 351: note = X may be A, G, I, L, P, or V
  VARIANT 387: note = X may be N, C, Q, M, S, or T
MNTINIAKNDFSDIELAAIPFNTLADHYGERLAREQLALEHESYEMGEARFRKMFERQLKAGEVADNAAAKPLITTLLPKMIARINDWFEEVKAKRGKRPTAFQFLQEIKPEAVAYITIKTTLACLTSADNTTVQAVASAIGRAIEDEARFGRIRDLEAKHFKKNVEEQLNKRVGHVYKKAFMQVVEADMLSKGLLGGEAWSSWHKEDSIHVGVRCIEMLIESTGMVSLHRQNAGVVGQDSETIELAPEYAEAIATRAGALAGISPMFQPCVVPPKPWTGITGGGYWANGRRPLALVRTHSKKALMRYEDVYMPEVYKAINIAQNTAWKINKKVLAVANVITKWKHCPVXXIPAIEREELPMKPEDIDMNPEALTAWKRAAAAVYRXDKARKSRRISLEFMLEQANKFANHKAIWFPYNMDWRGRVYAVSMFNPQGNDMTKGLLTLAKGKPIGKEGYYWLKIHGANCAGVDKVPFPERIKFIEENHENIMACAKSPLENTWWAEQDSPFCFLAFCFEYAGVQHHGLSYNCSLPLAFDGSCSGIQHFSAMLRDEVGGRAVNLLPSETVQDIYGIVAKKVNEILQADAINGTDNEVVTVTDENTGEISEKVKLGTKALAGQWLAYGVTRSVTKRSVMTLAYGSKEFGFRQQVLEDTIQPAIDSGKGLMFTQPNQAAGYMAKLIWESVSVTVVAAVEAMNWLKSAAKLLAAEVKDKKTGEILRKRCAVHWVTPDGFPVWQEYKKPIQTRLNLMFLGQFRLQPTINTNKDSEIDAHKQESGIAPNFVHSQDGSHLRKTVVWAHEKYGIESFALIHDSFGTIPADAANLFKAVRETMVDTYESCDVLADFYDQFADQLHESQLDKMPALPAKGNLNLRDILESDFAFA

SEQ ID NO: 5 - protein, 883 aa, synthetic construct
  VARIANT 350: note = X may be W, Y, or F
  VARIANT 351: note = X may be A, G, I, L, P, or V
  VARIANT 653: note = X may be W, Y, or F
MNTINIAKNDFSDIELAAIPFNTLADHYGERLAREQLALEHESYEMGEARFRKMFERQLKAGEVADNAAAKPLITTLLPKMIARINDWFEEVKAKRGKRPTAFQFLQEIKPEAVAYITIKTTLACLTSADNTTVQAVASAIGRAIEDEARFGRIRDLEAKHFKKNVEEQLNKRVGHVYKKAFMQVVEADMLSKGLLGGEAWSSWHKEDSIHVGVRCIEMLIESTGMVSLHRQNAGVVGQDSETIELAPEYAEAIATRAGALAGISPMFQPCVVPPKPWTGITGGGYWANGRRPLALVRTHSKKALMRYEDVYMPEVYKAINIAQNTAWKINKKVLAVANVITKWKHCPVXXIPAIEREELPMKPEDIDMNPEALTAWKRAAAAVYRKDKARKSRRISLEFMLEQANKFANHKAIWFPYNMDWRGRVYAVSMFNPQGNDMTKGLLTLAKGKPIGKEGYYWLKIHGANCAGVDKVPFPERIKFIEENHENIMACAKSPLENTWWAEQDSPFCFLAFCFEYAGVQHHGLSYNCSLPLAFDGSCSGIQHFSAMLRDEVGGRAVNLLPSETVQDIYGIVAKKVNEILQADAINGTDNEVVTVTDENTGEISEKVKLGTKALAGQWLAYGVTRSVTKRSVMTLAYGSKEFGFRQQVLEXTIQPAIDSGKGLMFTQPNQAAGYMAKLIWESVSVTVVAAVEAMNWLKSAAKLLAAEVKDKKTGEILRKRCAVHWVTPDGFPVWQEYKKPIQTRLNLMFLGQFRLQPTINTNKDSEIDAHKQESGIAPNFVHSQDGSHLRKTVVWAHEKYGIESFALIHDSFGTIPADAANLFKAVRETMVDTYESCDVLADFYDQFADQLHESQLDKMPALPAKGNLNLRDILESDFAFA

SEQ ID NO: 6 - protein, 883 aa, synthetic construct
MNTINIAKNDFSDIELAAIPFNTLADHYGERLAREQLALEHESYEMGEARFRKMFERQLKAGEVADNAAAKPLITTLLPKMIARINDWFEEVKAKRGKRPTAFQFLQEIKPEAVAYITIKTTLACLTSADNTTVQAVASAIGRAIEDEARFGRIRDLEAKHFKKNVEEQLNKRVGHVYKKAFMQVVEADMLSKGLLGGEAWSSWHKEDSIHVGVRCIEMLIESTGMVSLHRQNAGVVGQDSETIELAPEYAEAIATRAGALAGISPMFQPCVVPPKPWTGITGGGYWANGRRPLALVRTHSKKALMRYEDVYMPEVYKAINIAQNTAWKINKKVLAVANVITKWKHCPVWVIPAIEREELPMKPEDIDMNPEALTAWKRAAAAVYRSDKARKSRRISLEFMLEQANKFANHKAIWFPYNMDWRGRVYAVSMFNPQGYDMTKGLLTLAKGKPIGKEGYYWLKIHGANCAGVDKVPFPERIKFIEENHENIMACAKSPLENTWWAEQDSPFCFLAFCFEYAGVQHHGLSYNCSLPLAFDGSCSGIQHFSAMLRDEVGGRAVNLLPSETVQDIYGIVAKKVNEILQADAINGTDNEVVTVTDENTGEISEKVKLGTKALAGQWLAYGVTRSVTKRSVMTLAYGSKEFGFRQQVLEDTIQPAIDSGKGLMFTQPNQAAGYMAKLIWESVSVTVVAAVEAMNWLKSAAKLLAAEVKDKKTGEILRKRCAVHWVTPDGFPVWQEYKKPIQTRLNLMFLGQFRLQPTINTNKDSEIDAHKQESGIAPNFVHSQDGSHLRKTVVWAHEKYGIESFALIHDSFGTIPADAANLFKAVRETMVDTYESCDVLADFYDQFADQLHESQLDKMPALPAKGNLNLRDILESDFAFA

SEQ ID NO: 7 - protein, 883 aa, synthetic construct
MNTINIAKNDFSDIELAAIPFNTLADHYGERLAREQLALEHESYEMGEARFRKMFERQLKAGEVADNAAAKPLITTLLPKMIARINDWFEEVKAKRGKRPTAFQFLQEIKPEAVAYITIKTTLACLTSADNTTVQAVASAIGRAIEDEARFGRIRDLEAKHFKKNVEEQLNKRVGHVYKKAFMQVVEADMLSKGLLGGEAWSSWHKEDSIHVGVRCIEMLIESTGMVSLHRQNAGVVGQDSETIELAPEYAEAIATRAGALAGISPMFQPCVVPPKPWTGITGGGYWANGRRPLALVRTHSKKALMRYEDVYMPEVYKAINIAQNTAWKINKKVLAVANVITKWKHCPVWVIPAIEREELPMKPEDIDMNPEALTAWKRAAAAVYRKDKARKSRRISLEFMLEQANKFANHKAIWFPYNMDWRGRVYAVSMFNPQGYDMTKGLLTLAKGKPIGKEGYYWLKIHGANCAGVDKVPFPERIKFIEENHENIMACAKSPLENTWWAEQDSPFCFLAFCFEYAGVQHHGLSYNCSLPLAFDGSCSGIQHFSAMLRDEVGGRAVNLLPSETVQDIYGIVAKKVNEILQADAINGTDNEVVTVTDENTGEISEKVKLGTKALAGQWLAYGVTRSVTKRSVMTLAYGSKEFGFRQQVLEDTIQPAIDSGKGLMFTQPNQAAGYMAKLIWESVSVTVVAAVEAMNWLKSAAKLLAAEVKDKKTGEILRKRCAVHWVTPDGFPVWQEYKKPIQTRLNLMFLGQFRLQPTINTNKDSEIDAHKQESGIAPNFVHSQDGSHLRKTVVWAHEKYGIESFALIHDSFGTIPADAANLFKAVRETMVDTYESCDVLADFYDQFADQLHESQLDKMPALPAKGNLNLRDILESDFAFA

SEQ ID NO: 8 - protein, 883 aa, synthetic construct
MNTINIAKNDFSDIELAAIPFNTLADHYGERLAREQLALEHESYEMGEARFRKMFERQLKAGEVADNAAAKPLITTLLPKMIARINDWFEEVKAKRGKRPTAFQFLQEIKPEAVAYITIKTTLACLTSADNTTVQAVASAIGRAIEDEARFGRIRDLEAKHFKKNVEEQLNKRVGHVYKKAFMQVVEADMLSKGLLGGEAWSSWHKEDSIHVGVRCIEMLIESTGMVSLHRQNAGVVGQDSETIELAPEYAEAIATRAGALAGISPMFQPCVVPPKPWTGITGGGYWANGRRPLALVRTHSKKALMRYEDVYMPEVYKAINIAQNTAWKINKKVLAVANVITKWKHCPVWVIPAIEREELPMKPEDIDMNPEALTAWKRAAAAVYRSDKARKSRRISLEFMLEQANKFANHKAIWFPYNMDWRGRVYAVSMFNPQGNDMTKGLLTLAKGKPIGKEGYYWLKIHGANCAGVDKVPFPERIKFIEENHENIMACAKSPLENTWWAEQDSPFCFLAFCFEYAGVQHHGLSYNCSLPLAFDGSCSGIQHFSAMLRDEVGGRAVNLLPSETVQDIYGIVAKKVNEILQADAINGTDNEVVTVTDENTGEISEKVKLGTKALAGQWLAYGVTRSVTKRSVMTLAYGSKEFGFRQQVLEDTIQPAIDSGKGLMFTQPNQAAGYMAKLIWESVSVTVVAAVEAMNWLKSAAKLLAAEVKDKKTGEILRKRCAVHWVTPDGFPVWQEYKKPIQTRLNLMFLGQFRLQPTINTNKDSEIDAHKQESGIAPNFVHSQDGSHLRKTVVWAHEKYGIESFALIHDSFGTIPADAANLFKAVRETMVDTYESCDVLADFYDQFADQLHESQLDKMPALPAKGNLNLRDILESDFAFA

SEQ ID NO: 9 - protein, 883 aa, synthetic construct
MNTINIAKNDFSDIELAAIPFNTLADHYGERLAREQLALEHESYEMGEARFRKMFERQLKAGEVADNAAAKPLITTLLPKMIARINDWFEEVKAKRGKRPTAFQFLQEIKPEAVAYITIKTTLACLTSADNTTVQAVASAIGRAIEDEARFGRIRDLEAKHFKKNVEEQLNKRVGHVYKKAFMQVVEADMLSKGLLGGEAWSSWHKEDSIHVGVRCIEMLIESTGMVSLHRQNAGVVGQDSETIELAPEYAEAIATRAGALAGISPMFQPCVVPPKPWTGITGGGYWANGRRPLALVRTHSKKALMRYEDVYMPEVYKAINIAQNTAWKINKKVLAVANVITKWKHCPVWVIPAIEREELPMKPEDIDMNPEALTAWKRAAAAVYRKDKARKSRRISLEFMLEQANKFANHKAIWFPYNMDWRGRVYAVSMFNPQGNDMTKGLLTLAKGKPIGKEGYYWLKIHGANCAGVDKVPFPERIKFIEENHENIMACAKSPLENTWWAEQDSPFCFLAFCFEYAGVQHHGLSYNCSLPLAFDGSCSGIQHFSAMLRDEVGGRAVNLLPSETVQDIYGIVAKKVNEILQADAINGTDNEVVTVTDENTGEISEKVKLGTKALAGQWLAYGVTRSVTKRSVMTLAYGSKEFGFRQQVLEWTIQPAIDSGKGLMFTQPNQAAGYMAKLIWESVSVTVVAAVEAMNWLKSAAKLLAAEVKDKKTGEILRKRCAVHWVTPDGFPVWQEYKKPIQTRLNLMFLGQFRLQPTINTNKDSEIDAHKQESGIAPNFVHSQDGSHLRKTVVWAHEKYGIESFALIHDSFGTIPADAANLFKAVRETMVDTYESCDVLADFYDQFADQLHESQLDKMPALPAKGNLNLRDILESDFAFA

SEQ ID NO: 10 - protein, 892 aa, synthetic construct
MHHHHHHVNSNTINIAKNDFSDIELAAIPFNTLADHYGERLAREQLALEHESYEMGEARFRKMFERQLKAGEVADNAAAKPLITTLLPKMIARINDWFEEVKAKRGKRPTAFQFLQEIKPEAVAYITIKTTLACLTSADNTTVQAVASAIGRAIEDEARFGRIRDLEAKHFKKNVEEQLNKRVGHVYKKAFMQVVEADMLSKGLLGGEAWSSWHKEDSIHVGVRCIEMLIESTGMVSLHRQNAGVVGQDSETIELAPEYAEAIATRAGALAGISPMFQPCVVPPKPWTGITGGGYWANGRRPLALVRTHSKKALMRYEDVYMPEVYKAINIAQNTAWKINKKVLAVANVITKWKHCPVWVIPAIEREELPMKPEDIDMNPEALTAWKRAAAAVYRSDKARKSRRISLEFMLEQANKFANHKAIWFPYNMDWRGRVYAVSMFNPQGYDMTKGLLTLAKGKPIGKEGYYWLKIHGANCAGVDKVPFPERIKFIEENHENIMACAKSPLENTWWAEQDSPFCFLAFCFEYAGVQHHGLSYNCSLPLAFDGSCSGIQHFSAMLRDEVGGRAVNLLPSETVQDIYGIVAKKVNEILQADAINGTDNEVVTVTDENTGEISEKVKLGTKALAGQWLAYGVTRSVTKRSVMTLAYGSKEFGFRQQVLEDTIQPAIDSGKGLMFTQPNQAAGYMAKLIWESVSVTVVAAVEAMNWLKSAAKLLAAEVKDKKTGEILRKRCAVHWVTPDGFPVWQEYKKPIQTRLNLMFLGQFRLQPTINTNKDSEIDAHKQESGIAPNFVHSQDGSHLRKTVVWAHEKYGIESFALIHDSFGTIPADAANLFKAVRETMVDTYESCDVLADFYDQFADQLHESQLDKMPALPAKGNLNLRDILESDFAFA

SEQ ID NO: 11 - protein, 892 aa, synthetic construct
MHHHHHHVNSNTINIAKNDFSDIELAAIPFNTLADHYGERLAREQLALEHESYEMGEARFRKMFERQLKAGEVADNAAAKPLITTLLPKMIARINDWFEEVKAKRGKRPTAFQFLQEIKPEAVAYITIKTTLACLTSADNTTVQAVASAIGRAIEDEARFGRIRDLEAKHFKKNVEEQLNKRVGHVYKKAFMQVVEADMLSKGLLGGEAWSSWHKEDSIHVGVRCIEMLIESTGMVSLHRQNAGVVGQDSETIELAPEYAEAIATRAGALAGISPMFQPCVVPPKPWTGITGGGYWANGRRPLALVRTHSKKALMRYEDVYMPEVYKAINIAQNTAWKINKKVLAVANVITKWKHCPVWVIPAIEREELPMKPEDIDMNPEALTAWKRAAAAVYRKDKARKSRRISLEFMLEQANKFANHKAIWFPYNMDWRGRVYAVSMFNPQGYDMTKGLLTLAKGKPIGKEGYYWLKIHGANCAGVDKVPFPERIKFIEENHENIMACAKSPLENTWWAEQDSPFCFLAFCFEYAGVQHHGLSYNCSLPLAFDGSCSGIQHFSAMLRDEVGGRAVNLLPSETVQDIYGIVAKKVNEILQADAINGTDNEVVTVTDENTGEISEKVKLGTKALAGQWLAYGVTRSVTKRSVMTLAYGSKEFGFRQQVLEDTIQPAIDSGKGLMFTQPNQAAGYMAKLIWESVSVTVVAAVEAMNWLKSAAKLLAAEVKDKKTGEILRKRCAVHWVTPDGFPVWQEYKKPIQTRLNLMFLGQFRLQPTINTNKDSEIDAHKQESGIAPNFVHSQDGSHLRKTVVWAHEKYGIESFALIHDSFGTIPADAANLFKAVRETMVDTYESCDVLADFYDQFADQLHESQLDKMPALPAKGNLNLRDILESDFAFA

SEQ ID NO: 12 - protein, 892 aa, synthetic construct
MHHHHHHVNSNTINIAKNDFSDIELAAIPFNTLADHYGERLAREQLALEHESYEMGEARFRKMFERQLKAGEVADNAAAKPLITTLLPKMIARINDWFEEVKAKRGKRPTAFQFLQEIKPEAVAYITIKTTLACLTSADNTTVQAVASAIGRAIEDEARFGRIRDLEAKHFKKNVEEQLNKRVGHVYKKAFMQVVEADMLSKGLLGGEAWSSWHKEDSIHVGVRCIEMLIESTGMVSLHRQNAGVVGQDSETIELAPEYAEAIATRAGALAGISPMFQPCVVPPKPWTGITGGGYWANGRRPLALVRTHSKKALMRYEDVYMPEVYKAINIAQNTAWKINKKVLAVANVITKWKHCPVWVIPAIEREELPMKPEDIDMNPEALTAWKRAAAAVYRSDKARKSRRISLEFMLEQANKFANHKAIWFPYNMDWRGRVYAVSMFNPQGNDMTKGLLTLAKGKPIGKEGYYWLKIHGANCAGVDKVPFPERIKFIEENHENIMACAKSPLENTWWAEQDSPFCFLAFCFEYAGVQHHGLSYNCSLPLAFDGSCSGIQHFSAMLRDEVGGRAVNLLPSETVQDIYGIVAKKVNEILQADAINGTDNEVVTVTDENTGEISEKVKLGTKALAGQWLAYGVTRSVTKRSVMTLAYGSKEFGFRQQVLEDTIQPAIDSGKGLMFTQPNQAAGYMAKLIWESVSVTVVAAVEAMNWLKSAAKLLAAEVKDKKTGEILRKRCAVHWVTPDGFPVWQEYKKPIQTRLNLMFLGQFRLQPTINTNKDSEIDAHKQESGIAPNFVHSQDGSHLRKTVVWAHEKYGIESFALIHDSFGTIPADAANLFKAVRETMVDTYESCDVLADFYDQFADQLHESQLDKMPALPAKGNLNLRDILESDFAFA

SEQ ID NO: 13 - protein, 892 aa, synthetic construct
MHHHHHHVNSNTINIAKNDFSDIELAAIPFNTLADHYGERLAREQLALEHESYEMGEARFRKMFERQLKAGEVADNAAAKPLITTLLPKMIARINDWFEEVKAKRGKRPTAFQFLQEIKPEAVAYITIKTTLACLTSADNTTVQAVASAIGRAIEDEARFGRIRDLEAKHFKKNVEEQLNKRVGHVYKKAFMQVVEADMLSKGLLGGEAWSSWHKEDSIHVGVRCIEMLIESTGMVSLHRQNAGVVGQDSETIELAPEYAEAIATRAGALAGISPMFQPCVVPPKPWTGITGGGYWANGRRPLALVRTHSKKALMRYEDVYMPEVYKAINIAQNTAWKINKKVLAVANVITKWKHCPVWVIPAIEREELPMKPEDIDMNPEALTAWKRAAAAVYRKDKARKSRRISLEFMLEQANKFANHKAIWFPYNMDWRGRVYAVSMFNPQGNDMTKGLLTLAKGKPIGKEGYYWLKIHGANCAGVDKVPFPERIKFIEENHENIMACAKSPLENTWWAEQDSPFCFLAFCFEYAGVQHHGLSYNCSLPLAFDGSCSGIQHFSAMLRDEVGGRAVNLLPSETVQDIYGIVAKKVNEILQADAINGTDNEVVTVTDENTGEISEKVKLGTKALAGQWLAYGVTRSVTKRSVMTLAYGSKEFGFRQQVLEWTIQPAIDSGKGLMFTQPNQAAGYMAKLIWESVSVTVVAAVEAMNWLKSAAKLLAAEVKDKKTGEILRKRCAVHWVTPDGFPVWQEYKKPIQTRLNLMFLGQFRLQPTINTNKDSEIDAHKQESGIAPNFVHSQDGSHLRKTVVWAHEKYGIESFALIHDSFGTIPADAANLFKAVRETMVDTYESCDVLADFYDQFADQLHESQLDKMPALPAKGNLNLRDILESDFAFA

SEQ ID NO: 14 - protein, 6 aa, synthetic construct
HHHHHH

SEQ ID NO: 15 - protein, 7 aa, synthetic construct
HHHHHHV

SEQ ID NO: 16 - protein, 7 aa, synthetic construct
DYKDDDK

(19) United States
(12) Patent Application Publication
(10) Pub. No.: US 2025/0250551 A1
(43) Pub. Date: Aug. 7, 2025

(54) RNA POLYMERASE VARIANTS

(71) Applicant: ModernaTX, Inc., Cambridge, MA (US)

(72) Inventors: Amy E. Rabideau, Cambridge, MA (US); Margaret Franklin, Cambridge, MA (US); Athanasios Dousis, Cambridge, MA (US)

(73) Assignee: ModernaTX, Inc., Cambridge, MA (US)

(21) Appl. No.: 18/856,588

(22) PCT Filed: Apr. 13, 2023
(86) PCT No.: PCT/US2023/065719

§ 371 Date: Oct. 11, 2024

Related U.S. Application Data
(60) Provisional application No. 63/331,145, filed on Apr. 14, 2022.

(51) Int. Cl. C12N 9/12

(52) U.S. Cl. CPC C12N 9/1247; C12Y 207/07006

(57) ABSTRACT

RNA polymerase variants enable high efficiency transcription of RNA. In Yield some embodiments, the RNA polymerase variants enable RNA transcription with high capping efficiency and/or low levels of double-stranded RNA contamination.

### RELATED APPLICATION

This application claims the benefit under 35 U.S.C. § 119(e) of U.S. provisional application No. 63/331,145, filed Apr. 14, 2022, the contents of which are incorporated by reference herein in their entirety.

### REFERENCE TO AN ELECTRONIC SEQUENCE LISTING

The contents of the electronic Sequence Listing (M137870217WO00-SEQ-HJD.xml; Size: 31,370 bytes; and Date of Creation: Apr. 12, 2023) are herein incorporated by reference in their entirety.

### BACKGROUND

The emergence of ribonucleic acid (RNA)-based therapeutics requires a polymerase that produces RNA with few byproducts from aberrant activity. Transcripts resulting from in vitro transcription using the bacteriophage T7 RNA polymerase exhibit an immune-stimulatory activity that is often undesirable and uncontrollable. This immune-stimulatory activity of T7 transcript is contributed by its aberrant activity to initiate transcription from a promoter-less deoxyribonucleic acid (DNA) end. This activity results in the production of an antisense RNA that is fully complementary to the intended sense RNA product, and consequently a long double-stranded RNA (dsRNA) that can robustly stimulate an unintended immune response. Furthermore, the bacteriophage T7 RNA polymerase produces T7 transcripts having low 5′ end capping efficiency in the presence of cap analog(s), in part because the polymerase has low binding affinity for the cap analog(s).

### SUMMARY

Some aspects comprise T7 RNA polymerase variants and in vitro transcription methods using these variants, which have been shown to reduce dsRNA contaminant and/or improve co-transcriptional 5′ end capping efficiency, relative to a control (e.g., wild-type T7 RNA polymerase comprising the amino acid sequence of SEQ ID NO: 1).

Some aspects provide an ribonucleic acid (RNA) polymerase variant comprising an amino acid sequence having at least 90% sequence identity to the amino acid sequence of any one of SEQ ID NOs: 2-9, wherein the amino acid sequence comprises an amino acid substitution at position D351 and at least two additional amino acid substitutions, relative to a RNA polymerase comprising the amino acid sequence of SEQ ID NO: 1.

Some aspects provide an RNA polymerase variant comprising an amino acid sequence that comprises at least one, at least two, at least three, or at least four amino acid substitutions, relative to a wild-type T7 RNA polymerase (e.g., wild-type T7 RNA polymerase comprising the amino acid sequence of SEQ ID NO: 1).

Some aspects provide an RNA polymerase variant comprising an amino acid sequence having at least 90%, at least 95%, at least 98%, or 100% sequence identity to the amino acid sequence of any one of SEQ ID NOs: 2-9.

Some aspects provide an RNA polymerase variant comprising: an amino acid sequence comprising (i) an amino acid substitution at position E350, (ii) an amino acid substitution at D351, and (iii) an amino acid substitution at position K387, position N437, or at position K387 and position N437, relative to a wild-type T7 RNA polymerase comprising the amino acid sequence of SEQ ID NO: 1.

In some embodiments, the amino acid sequence of the variant comprises an amino acid substitution at position K387.

In some embodiments, the amino acid sequence of the variant comprises an amino acid substitution at position N437.

In some embodiments, the amino acid sequence of the variant comprises an amino acid substitution at position K387 and at position N437.

In some embodiments, the amino acid substitution at position K387 is a polar, neutral amino acid.

In some embodiments, the polar, neutral amino acid is selected from asparagine (N), cysteine (C), glutamine (Q), methionine (M), serine (S), and threonine (T).

In some embodiments, the polar, neutral amino acid is asparagine (K387N).

In some embodiments, the polar, neutral amino acid is cysteine (K387C).

In some embodiments, the polar, neutral amino acid is glutamine (K387Q).

In some embodiments, the polar, neutral amino acid is methionine (K387M).

In some embodiments, the polar, neutral amino acid is serine (K387S).

In some embodiments, the polar, neutral amino acid is threonine (K387T).

In some embodiments, the amino acid substitution at position N437 is an aromatic amino acid.

In some embodiments, the aromatic amino acid is selected from tryptophan (W), tyrosine (Y), and phenylalanine (F).

In some embodiments, the aromatic amino acid is tryptophan (N437W).

In some embodiments, the aromatic amino acid is tyrosine (N437Y).

In some embodiments, the aromatic amino acid is phenylalanine (N437F).

Other aspects provide an RNA polymerase variant comprising an amino acid sequence that comprises (i) an amino acid substitution at position E350, (ii) an amino acid substitution at D351, and (iii) an amino acid substitution at position D653, relative to a wild-type T7 RNA polymerase comprising the amino acid sequence of SEQ ID NO: 1.

In some embodiments, the amino acid substitution at position D653 is an aromatic amino acid.

In some embodiments, the aromatic amino acid is selected from tryptophan (W), tyrosine (Y), and phenylalanine (F).

In some embodiments, the aromatic amino acid is tryptophan (D653W).

In some embodiments, the aromatic amino acid is tyrosine (D653Y).

In some embodiments, the aromatic amino acid is phenylalanine (D653F).

In some embodiments, the amino acid substitution at position E350 is an aromatic amino acid.

In some embodiments, the aromatic amino acid is selected from tryptophan (W), tyrosine (Y), and phenylalanine (F).

In some embodiments, the aromatic amino acid is tryptophan (E350W).

In some embodiments, the aromatic amino acid is tyrosine (E350Y).

In some embodiments, the aromatic amino acid is phenylalanine (E350F).

In some embodiments, the amino acid substitution at position D351 is a non-polar, aliphatic amino acid.

In some embodiments, the non-polar, aliphatic amino acid is selected from alanine (A), glycine (G), isoleucine (I), leucine (L), proline (P), and valine (V).

In some embodiments, the non-polar, aliphatic amino acid is alanine (D351A).

In some embodiments, the non-polar, aliphatic amino acid is glycine (D351G).

In some embodiments, the non-polar, aliphatic amino acid is isoleucine (D351I).

In some embodiments, the non-polar, aliphatic amino acid is leucine (D351L).

In some embodiments, the non-polar, aliphatic amino acid is proline (D351P).

In some embodiments, the non-polar, aliphatic amino acid is valine (D351V).

Yet other aspects provide an RNA polymerase variant comprising: an amino acid sequence having at least 70% identity to the amino acid sequence of SEQ ID NO: 1, wherein the amino acid sequence of the variant comprises (i) an amino acid substitution at position E350, (ii) an amino acid substitution at D351, and (iii) an amino acid substitution at position K387, position N437, or at position K387 and position N437, relative to a wild-type T7 RNA polymerase comprising the amino acid sequence of SEQ ID NO: 1.

In some embodiments, the amino acid sequence has at least 75%, at least 80%, at least 85%, at least 95%, or at least 98% identity to the amino acid sequence of SEQ ID NO: 1.

In some embodiments, the amino acid sequence of the variant comprises an amino acid substitution at position K387.

In some embodiments, the amino acid sequence of the variant comprises an amino acid substitution at position N437.

In some embodiments, the amino acid sequence of the variant comprises an amino acid substitution at position K387 and at position N437.

In some embodiments, the amino acid substitution at position K387 is a polar, neutral amino acid.

In some embodiments, the polar, neutral amino acid is selected from asparagine (K387N), cysteine (K387C), glutamine (K387Q), methionine (K387M), serine (K387S), and threonine (K387T).

In some embodiments, the amino acid substitution at position N437 is an aromatic amino acid.

In some embodiments, the aromatic amino acid is selected from tryptophan (N437W), tyrosine (N437Y), and phenylalanine (N437F).

Still other aspects provide an RNA polymerase variant comprising: an amino acid sequence having at least 70% identity to the amino acid sequence of SEQ ID NO: 1, wherein the amino acid sequence of the variant comprises (i) an amino acid substitution at position E350, (ii) an amino acid substitution at D351, and (iii) an amino acid substitution at position D653, relative to a wild-type T7 RNA polymerase comprising the amino acid sequence of SEQ ID NO: 1.

In some embodiments, the amino acid sequence has at least 75%, at least 80%, at least 85%, at least 95%, or at least 98% identity to the amino acid sequence of SEQ ID NO: 1.

In some embodiments, the amino acid substitution at position D653 is an aromatic amino acid.

In some embodiments, the aromatic amino acid is selected from tryptophan (D653W), tyrosine (D653Y), and phenylalanine (D653F).

In some embodiments, the amino acid substitution at position E350 is an aromatic amino acid.

In some embodiments, the aromatic amino acid is selected from tryptophan (E350W), tyrosine (E350Y), and phenylalanine (E350F).

In some embodiments, the amino acid substitution at position D351 is a non-polar, aliphatic amino acid.

In some embodiments, the non-polar, aliphatic amino acid is selected from alanine (D351A), glycine (D351G), isoleucine (D351I), leucine (D351L), proline (D351P), and valine (D351V).

Some aspects provide an RNA polymerase variant comprising the amino acid sequence of SEQ ID NO: 2, wherein X¹ is an aromatic amino acid, optionally selected from W, Y, and F; X² is selected from a non-polar, aliphatic amino acid, optionally selected from A, G, I, L, P, and V; X³ is a polar, neutral amino acid, optionally selected from N, C, Q, M, S, and T; and X⁴ is an aromatic amino acid, optionally selected from W, Y, and F. In some embodiments, an RNA polymerase variant comprises the amino acid sequence of SEQ ID NO: 6.

Some aspects provide an RNA polymerase variant comprising the amino acid sequence of SEQ ID NO: 3, wherein X¹ is an aromatic amino acid, optionally selected from W, Y, and F; X² is selected from a non-polar, aliphatic amino acid, optionally selected from A, G, I, L, P, and V; and X⁴ is an aromatic amino acid, optionally selected from W, Y, and F. In some embodiments, an RNA polymerase variant comprises the amino acid sequence of SEQ ID NO: 7.

Some aspects provide an RNA polymerase variant comprising the amino acid sequence of SEQ ID NO: 4, wherein X¹ is an aromatic amino acid, optionally selected from W, Y, and F; X² is selected from a non-polar, aliphatic amino acid, optionally selected from A, G, I, L, P, and V; and X³ is a polar, neutral amino acid, optionally selected from N, C, Q, M, S, and T. In some embodiments, an RNA polymerase variant comprises the amino acid sequence of SEQ ID NO: 8.

Some aspects provide an RNA polymerase variant comprising the amino acid sequence of SEQ ID NO: 5, wherein X¹ is an aromatic amino acid, optionally selected from W, Y, and F; X² is selected from a non-polar, aliphatic amino acid, optionally selected from A, G, I, L, P, and V; and X⁵ is an aromatic amino acid, optionally selected from W, Y, and F. In some embodiments, an RNA polymerase variant comprises the amino acid sequence of SEQ ID NO: 9.

Some aspects provide a method comprising: producing a messenger RNA (mRNA) in an in vitro transcription reaction that comprises a DNA, nucleoside triphosphates, the RNA polymerase variant of any one of the preceding paragraphs, and optionally a cap analog.

In some embodiments, the reaction comprises the cap analog.

In some embodiments, the cap analog is a dinucleotide cap analog, a trinucleotide cap analog, or a tetranucleotide cap analog. In some embodiments, the cap analog is a tetranucleotide cap analog.

In some embodiments, the cap analog is a trinucleotide cap analog comprising a GAG sequence. In some embodiments, the GAG cap analog comprises a compound selected from:

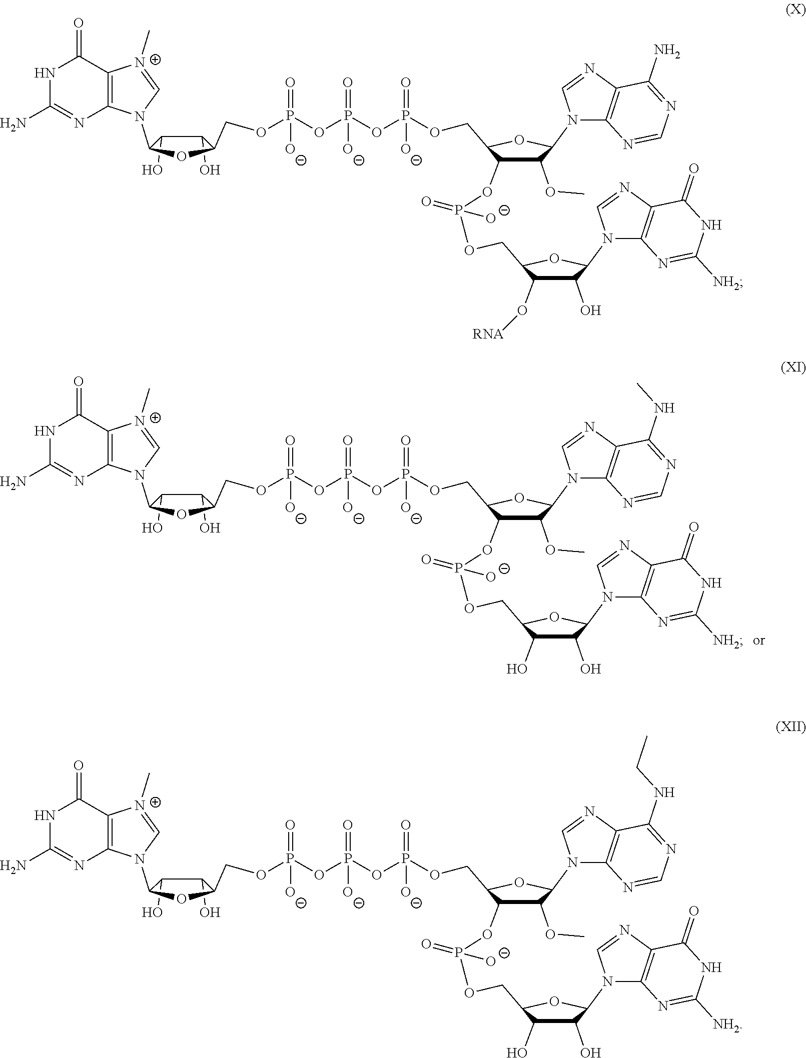

In some embodiments, the tetranucleotide cap analog comprises a GGAG sequence. In some embodiments, the tetranucleotide cap analog comprises a compound selected from:

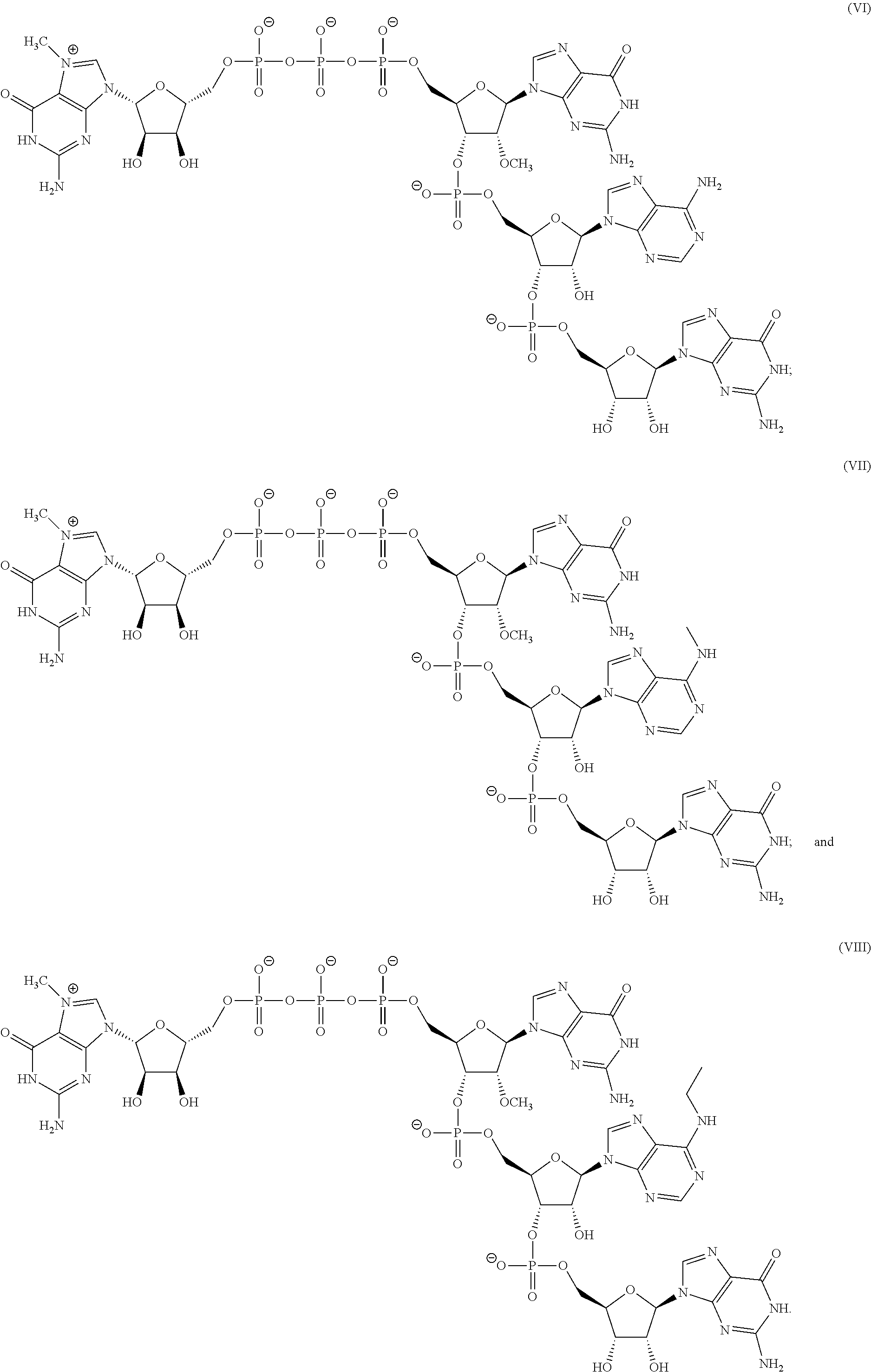

In some embodiments, the DNA includes a 2′-deoxythymidine residue or a 2′-deoxycytidine residue at position +1.

Some aspects include a composition or kit comprising the RNA polymerase variant of any one of the preceding paragraphs and an in vitro transcription (IVT) reagent selected from the group consisting of a DNA, nucleoside triphosphates, and a cap analog.

Some aspects include a nucleic acid encoding the RNA polymerase variant of any one of the preceding paragraphs.

### BRIEF DESCRIPTION OF THE DRAWINGS

FIGS. 1A-1D show graphs depicting the functional characteristics of transcribed RNA products resulting from in vitro transcription (IVT) reactions involving exemplary RNA polymerase variants. Following an oligo dT purification, transcribed RNA products were analyzed for yield (FIG. 1A), percent capped RNA (FIG. 1B), percent tailed (i.e., percent of RNA comprising a polyA tail) according to a Tris RP (reverse-phase) method (FIG. 1C), and amount of dsRNA (FIG. 1D).

FIGS. 2A-2C show graphs depicting the functional characteristics of transcribed RNA products resulting from in vitro transcription (IVT) reactions involving exemplary RNA polymerase variants in the presence of varying levels of GGAG cap analog. Following an oligo dT purification, transcribed RNA products were analyzed for percent capped RNA (FIG. 2A), yield (FIG. 2B), and percent tailed (i.e., percent of RNA comprising a polyA tail) according to a Tris RP (reverse-phase) method (FIG. 2C).

### DETAILED DESCRIPTION

RNA polymerase (e.g., DNA-dependent RNA polymerase) is an enzyme that catalyzes the sequential addition of a ribonucleotide to the 3′ end of a growing RNA chain (transcription of RNA in the 5′→3′ direction), with nucleoside triphosphates (NTPs) acting as substrates for the enzyme and with the sequence of nucleotides specified by a DNA template. Transcription relies on the complementary pairing of bases. The two strands of a double helix separate locally, and one of the separated strands serves as a template (DNA template). RNA polymerase then catalyzes the alignment of free nucleotides on the DNA template by their complementary bases in the template. Thus, an RNA polymerase is considered to have RNA polymerase activity if the polymerase catalyzes the sequential addition of a ribonucleotide to the 3′ end of a growing RNA chain.

DNA-directed RNA polymerases are capable of initiating synthesis of RNA without primers; the first catalytic stage of initiation is referred to as de novo RNA synthesis. De novo synthesis is a unique phase in the transcription cycle where the RNA polymerase binds two nucleotides rather than a nascent RNA polymer and a single nucleotide. For bacteriophage T7 RNA polymerase, transcription begins with a marked preference for GTP at the +1 and +2 positions. Initiating nucleotides bind RNA polymerase in locations distinct from those described for elongation complexes (Kennedy W P et al. J Mol Biol. 2007; 370(2): 256-68). Selection bias in favor of GTP as an initiating nucleotide is achieved by shape complementarity, extensive protein side-chain, and strong base-stacking interactions for the guanine moiety in the enzyme active site. Thus, an initiating GTP provides the largest stabilization force for the open promoter conformation (Kennedy et al. 2007). The RNA polymerase variants, in some embodiments, comprise one or more amino acid substitution(s) at one or more binding site residue(s) for de novo RNA synthesis, which, without being bound by theory, alters RNA polymerase affinity to the cap analog of an in vitro transcription reaction, for example, such that there is an improvement in capping efficiency at low cap analog concentrations.

Thus, in some aspects, RNA polymerase variants comprise an RNA polymerase that includes two or more amino acid substitutions at binding site residues for de novo RNA synthesis. An RNA polymerase variant is an enzyme having RNA polymerase activity and at least one substitution and/or modification relative to the counterpart wild-type RNA polymerase. In some embodiments, the amino acid substitution is at a position selected from positions 350, 351, 387, 437, and 653, relative to the wild-type RNA polymerase, wherein the wild-type RNA polymerase comprises the amino acid sequence of SEQ ID NO: 1.

Structural studies of T7 RNA polymerase have shown that the conformation of the N-terminal domain changes substantially between the initiation phase and elongation phase of transcription. The N-terminal domain comprises a C-helix subdomain and the promoter binding domain, which includes two segments separated by subdomain H. The promoter binding domain and the bound promoter rotate by approximately 45 degrees upon synthesis of an 8-nt RNA transcript, allowing the promoter contacts to be maintained while the active site is expanded to accommodate a growing heteroduplex. The C-helix subdomain moves modestly toward its elongation conformation, whereas subdomain H remains in its initiation—rather than its elongation-phase location, more than 70 angstroms away. Comparison of the structures of the T7 RNA polymerase initiation and elongation complexes reveal extensive conformational changes within the N-terminal 267 residues (N-terminal domain) and little change in the rest of the RNA polymerase. A rigid body rotation of the promoter binding domain as well as the refolding of the N-terminal C-helix (residues 28-71) and H (residues 151-190) subdomains are responsible for abolishing the promoter binding site, enlarging the active site and creating an exit tunnel for the RNA transcript. In particular, residues E42-G47 of T7 RNA polymerase, which exist as a j-loop structure in the initiation complex, adopt an α-helical structure in the elongation complex. The structural changes within the N-terminal domain account for the increased stability and the processivity of the elongation complex (see, e.g., Durniak, K. J. et al., Science 322(5901): 553-557, 2008, incorporated herein by reference). T7 RNA polymerase also comprises an ‘N helix’ (residues 374-409) that functions to divert the direction of the 5′ end of RNA transcript as it separates from template and influences the stability and processivity of the elongation complex (e.g., through the interactions between residues 385-395 and the ribose backbone). The ‘O helix’ of the RNA polymerase (residues 627-640) functions to stabilize the incoming NTP during insertion and prevent backtracking during synthesis of the RNA transcript. Finally, the ‘Y helix’ (residues 644-661) functions to stabilize the template base at the n+1 position of the growing RNA transcript.

In some aspects are RNA polymerase variants (e.g., T7 RNA polymerase variants) that facilitate the conformational change from the RNA polymerase initiation complex to the RNA polymerase elongation complex. In some embodiments, an RNA polymerase variant comprises at least one, at least two, at least three, or at least four amino acid modifications, relative to wild-type RNA polymerase, that causes at least one three-dimensional loop structure of the RNA polymerase variant to undergo a conformational change to a helix structure as the RNA polymerase variant transitions from an initiation complex to an elongation complex. Thus, in some embodiments, at least one amino acid modification has a high-helix propensity, relative to wild-type amino acid.

Furthermore, in some aspects are RNA polymerase variants (e.g., T7 RNA polymerase variants) that increase stability and processivity of the elongation complex, prevent backtracking and stabilize the incoming NTPs and template, relative to wild-type T7 RNA polymerase. In some embodiments, an RNA polymerase variant comprises at least one, at least two, at least three, or at least four amino acid modifications, relative to wild-type RNA polymerase, that increase stability and processivity of the elongation complex, prevent backtracking and stabilize the incoming NTPs and template. In some embodiments, an RNA polymerase variant comprises at least one, at least two, at least three, or at least four amino acid modifications, relative to wild-type RNA polymerase, in the ‘N helix’ (residues 374-409) (e.g., to increase stability and processivity of the elongation complex). In some embodiments, an RNA polymerase variant comprises at least one, at least two, at least three, or at least four amino acid modifications, relative to wild-type RNA polymerase, in the ‘O helix’ (residues 627-640) (e.g., to stabilize the incoming NTP during insertion and prevent backtracking). In some embodiments, an RNA polymerase variant comprises at least one, at least two, at least three, or at least four amino acid modifications, relative to wild-type RNA polymerase, in the ‘Y helix’ (residues 644-661) (e.g., to stabilize the growing RNA transcript).

Thus, some aspects provide RNA polymerase variants that comprise multiple amino acid substitutions and/or modifications, relative to wild-type RNA polymerase. In some embodiments, an RNA polymerase variant comprises an amino acid sequence that includes (a) an amino acid substitution at a binding site residue for de novo RNA synthesis, and (b) an amino acid substitution that facilitates the conformational change from the RNA polymerase initiation complex to the RNA polymerase elongation complex.

Use of the RNA polymerase variants in an in vitro transcription reaction, in some embodiments, increases transcription efficiency, relative to a control RNA polymerase. For example, use of an RNA polymerase variant may increase the transcription efficiency (e.g., RNA yield and/or rate of transcription) by at least 20%. In some embodiments, use of an RNA polymerase variant increases the transcription efficiency (e.g., RNA yield and/or rate of transcription) by at least 30%, at least 40%, at least 50%, at least 60%, at least 70%, at least 80%, at least 90%, or at least 10%. In some embodiments, use of an RNA polymerase variant increases the transcription efficiency by 20-100%, 20-90%, 20-80%, 20-70%, 20-60%, 20-50%, 30-100%, 30-90%, 30-80%, 30-70%, 30-60%, 30-50%, 40-100%, 40-90%, 40-80%, 40-70%, 40-60%, 40-50%, 50-100%, 50-90%, 50-80%, 50-70%, or 50-60%. In some embodiments, use of an RNA polymerase variant increases the total RNA yield by at least 30%, at least 40%, at least 50%, at least 60%, at least 70%, at least 80%, at least 90%, or at least 10%. In some embodiments, use of an RNA polymerase variant increases the total RNA yield by 20-100%, 20-90%, 20-80%, 20-70%, 20-60%, 20-50%, 30-100%, 30-90%, 30-80%, 30-70%, 30-60%, 30-50%, 40-100%, 40-90%, 40-80%, 40-70%, 40-60%, 40-50%, 50-100%, 50-90%, 50-80%, 50-70%, or 50-60%. In some embodiments, use of an RNA polymerase variant increases the rate of transcription by at least 30%, at least 40%, at least 50%, at least 60%, at least 70%, at least 80%, at least 90%, or at least 10%. In some embodiments, use of an RNA polymerase variant increases the rate of transcription by 20-100%, 20-90%, 20-80%, 20-70%, 20-60%, 20-50%, 30-100%, 30-90%, 30-80%, 30-70%, 30-60%, 30-50%, 40-100%, 40-90%, 40-80%, 40-70%, 40-60%, 40-50%, 50-100%, 50-90%, 50-80%, 50-70%, or 50-60%. In some embodiments, the control RNA polymerase is a wild-type RNA polymerase comprising the amino acid sequence of SEQ ID NO: 1 (“wild-type T7 RNA polymerase”).

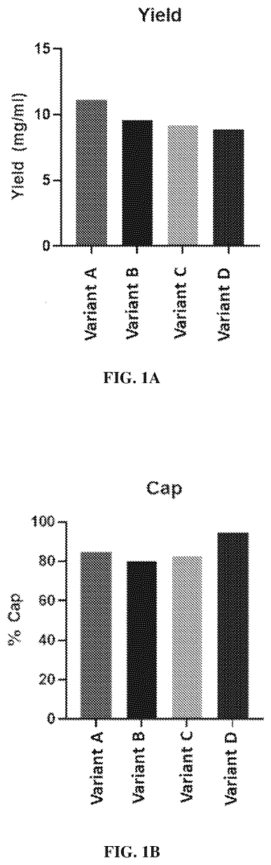

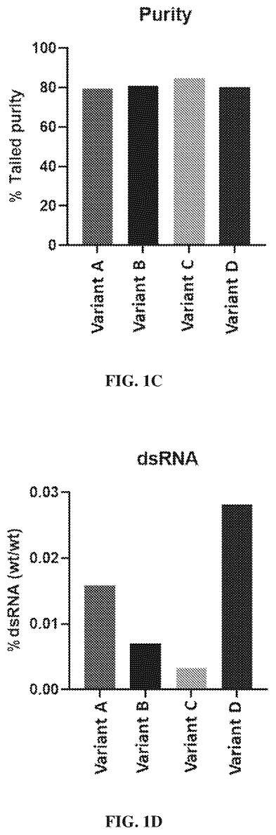

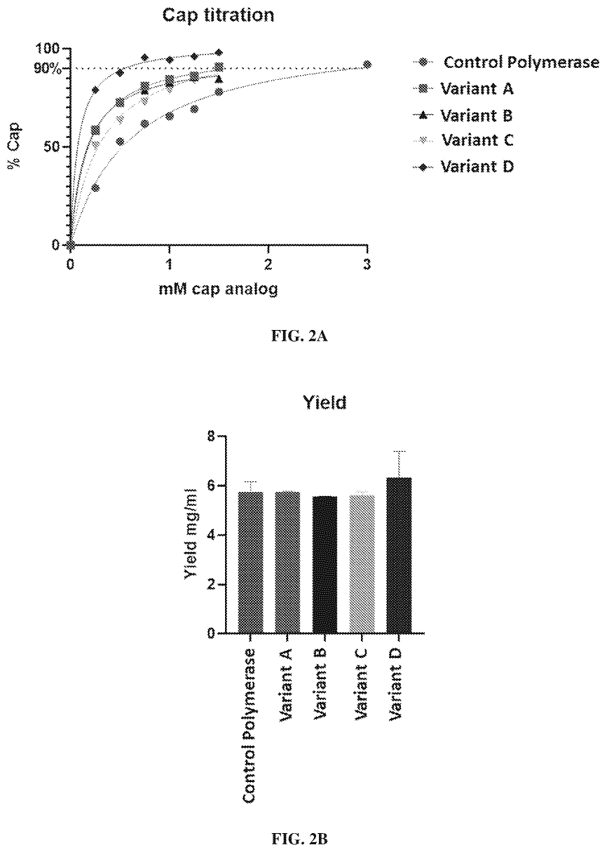

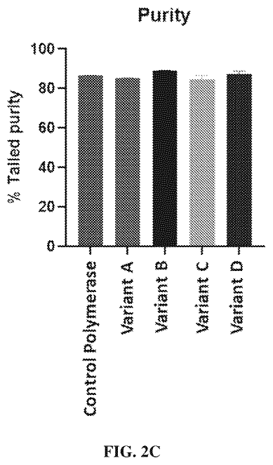

Surprisingly, RNA polymerase variants enable the use of a much lower concentration (amount) of cap analog in an in vitro transcription reaction to produce an amount of capped RNA equivalent to that produced using the wild-type T7 RNA polymerase. See, for example, FIGS. 1A-2C and Examples 1-2. In some embodiments, use of the RNA polymerase variants in an in vitro transcription reaction increases the yield of capped RNA when half the concentration of a cap analog is used in the in vitro transcription reaction. In some embodiments, use of the RNA polymerase variants in an in vitro transcription reaction increases the yield of capped RNA when only 25%, 50%, or 75% of the concentration of a cap analog is used in the in vitro transcription reaction. For example, use of an RNA polymerase variant may increase the yield of capped RNA by at least 20%, when only 25%, 50%, or 75% of the concentration of a cap analog is used in the in vitro transcription reaction. In some embodiments, use of an RNA polymerase variant increases the yield of capped RNA by at least 30%, at least 40%, at least 50%, at least 60%, at least 70%, at least 80%, at least 90%, or at least 100%, when only 25%, 50%, or 75% of the concentration of a cap analog is used in the in vitro transcription reaction. In some embodiments, use of an RNA polymerase variant increases the yield of capped RNA by 20-100%, 20-90%, 20-80%, 20-70%, 20-60%, 20-50%, 30-100%, 30-90%, 30-80%, 30-70%, 30-60%, 30-50%, 40-100%, 40-90%, 40-80%, 40-70%, 40-60%, 40-50%, 50-100%, 50-90%, 50-80%, 50-70%, or 50-60%, when only 25%, 50%, or 75% of the concentration of a cap analog is used in the in vitro transcription reaction. In some embodiments, the control RNA polymerase is a wild-type T7 RNA polymerase.

In some embodiments, use of an RNA polymerase variant increases the total yield of capped RNA by at least 30%, at least 40%, at least 50%, at least 60%, at least 70%, at least 80%, at least 90%, or at least 10%. In some embodiments, use of an RNA polymerase variant increases the total yield of capped RNA by 20-100%, 20-90%, 20-80%, 20-70%, 20-60%, 20-50%, 30-100%, 30-90%, 30-80%, 30-70%, 30-60%, 30-50%, 40-100%, 40-90%, 40-80%, 40-70%, 40-60%, 40-50%, 50-100%, 50-90%, 50-80%, 50-70%, or 50-60%.

In some embodiments, use of the RNA polymerase variants in an in vitro transcription reaction increases the co-transcriptional capping efficiency. For example, use of an RNA polymerase variant may increase the co-transcriptional capping efficiency (e.g., percentage of transcript comprising cap analog) by at least 20%. In some embodiments, use of an RNA polymerase variant increases the co-transcriptional capping efficiency (e.g., percentage of transcript comprising cap analog) by at least 30%, at least 40%, at least 50%, at least 60%, at least 70%, at least 80%, at least 90%, or at least 100%. In some embodiments, use of an RNA polymerase variant increases the co-transcriptional capping efficiency by 20-100%, 20-90%, 20-80%, 20-70%, 20-60%, 20-50%, 30-100%, 30-90%, 30-80%, 30-70%, 30-60%, 30-50%, 40-100%, 40-90%, 40-80%, 40-70%, 40-60%, 40-50%, 50-100%, 50-90%, 50-80%, 50-70%, or 50-60%. In some embodiments, the control RNA polymerase is a wild-type T7 RNA polymerase.

In some embodiments, at least 50% of the mRNA comprises a functional cap analog. For example, at least 50%, at least 55%, at least 60%, at least 65%, at least 70%, at least 75%, at least 80%, at least 85%, at least 95%, or 100% of the mRNA may comprise a cap analog. In some embodiments, 50%-100%, 50%-90%, 50%-80%, or 50%-70% of the mRNA comprises a cap analog.

In some embodiments, use of the RNA polymerase variants in an in vitro transcription reaction improves 3′ homogeneity of RNA at half the concentration of a cap analog used in the in vitro transcription reaction. For example, use of an RNA polymerase variant may improve 3′ homogeneity of RNA by at least 20%, when only 25%, 50%, or 75% of the concentration of a cap analog is used in the in vitro transcription reaction. In some embodiments, use of an RNA polymerase variant improves 3′ homogeneity by at least 30%, at least 40%, at least 50%, at least 60%, at least 70%, at least 80%, at least 90%, or at least 100%, when only 25%, 50%, or 75% of the concentration of a cap analog is used in the in vitro transcription reaction. In some embodiments, use of an RNA polymerase variant improves 3′ homogeneity by 20-100%, 20-90%, 20-80%, 20-70%, 20-60%, 20-50%, 30-100%, 30-90%, 30-80%, 30-70%, 30-60%, 30-50%, 40-100%, 40-90%, 40-80%, 40-70%, 40-60%, 40-50%, 50-100%, 50-90%, 50-80%, 50-70%, or 50-60%, when only 25%, 50%, or 75% of the concentration of a cap analog is used in the in vitro transcription reaction. In some embodiments, the control RNA polymerase is a wild-type T7 RNA polymerase.

In some embodiments, at least 50% of the mRNA produced in an in vitro transcription reaction that comprises an RNA polymerase variant exhibits 3′ homogeneity. For example, at least 55%, at least 60%, at least 65%, at least 70%, at least 75%, at least 80%, at least 85%, at least 95%, or 100% of the mRNA exhibits 3′ homogeneity. In some embodiments, 50%-100%, 50%-90%, 50%-80%, or 50%-70% of the mRNA exhibits 3′ homogeneity.

In some embodiments, the mRNA produced in an in vitro transcription reaction that comprises an RNA polymerase variant has greater than a threshold 3′ homogeneity. In some embodiments, the threshold is 50% or at least 50%. For example, the threshold may be 55%, 60%, 65%, 70%, 75%, 80%, 85%, or 90%.

In some embodiments, use of the RNA polymerase variants in an in vitro transcription reaction improves fidelity (e.g., mutation rate) of transcription. For example, use of an RNA polymerase variant may improve fidelity of transcription by at least 20%. In some embodiments, use of an RNA polymerase variant improves fidelity of transcription by at least 30%, at least 40%, at least 50%, at least 60%, at least 70%, at least 80%, at least 90%, or at least 100%. In some embodiments, use of an RNA polymerase variant improves fidelity of transcription by 20-100%, 20-90%, 20-80%, 20-70%, 20-60%, 20-50%, 30-100%, 30-90%, 30-80%, 30-70%, 30-60%, 30-50%, 40-100%, 40-90%, 40-80%, 40-70%, 40-60%, 40-50%, 50-100%, 50-90%, 50-80%, 50-70%, or 50-60%. An RNA polymerase variant that improves fidelity of transcription will produce RNA transcript (e.g., mRNA transcript) with a lower rate or total number of mutations than a control RNA polymerase. In some embodiments, the control RNA polymerase is a wild-type T7 RNA polymerase.

In some embodiments, the mRNA produced using an RNA polymerase variant has less than 1 mutation per 100 nucleotides relative to the DNA template. For example, the mRNA produced may have less than 1 mutation per 200, 300, 400, 500, 600, 700, 800, 900 or 1000 nucleotides relative to the DNA template.

In some embodiments, use of the RNA polymerase variants in an in vitro transcription reaction lowers the amount of double-stranded RNA (dsRNA) contamination in the in vitro transcription reaction. For example, use of an RNA polymerase variant may lower the amount of dsRNA contamination in the in vitro transcription reaction by at least 20%. In some embodiments, use of an RNA polymerase variant lowers the amount of dsRNA contamination in the in vitro transcription reaction by at least 30%, at least 40%, at least 50%, at least 60%, at least 70%, at least 80%, at least 90%, or at least 100%. In some embodiments, use of an RNA polymerase variant lowers the amount of dsRNA contamination in the in vitro transcription reaction by 20-100%, 20-90%, 20-80%, 20-70%, 20-60%, 20-50%, 30-100%, 30-90%, 30-80%, 30-70%, 30-60%, 30-50%, 40-100%, 40-90%, 40-80%, 40-70%, 40-60%, 40-50%, 50-100%, 50-90%, 50-80%, 50-70%, or 50-60%. In some embodiments, the control RNA polymerase is a wild-type T7 RNA polymerase.

In some embodiments, the concentration of dsRNA contamination is less than 10 ng per g of mRNA product. In some embodiments, the concentration of dsRNA contamination is less than 5 ng per 25 g of mRNA product. For example, the concentration of dsRNA contamination may be less than 4 ng per 25 g of mRNA product, less than 3 ng per 25 g of mRNA product, less than 2 ng per 25 g of mRNA product, or less than less than 1 ng per 25 g of mRNA product. In some embodiments, the concentration of dsRNA contamination is 0.5-1, 0.5-2, 0.5-3, 0-0.4, or 0.5-5 ng per 25 g of mRNA product.

In some embodiments, the mRNA produced in an in vitro transcription reaction that comprises an RNA polymerase variant has lower than a threshold quantity of dsRNA. In some embodiments, the threshold is 10 ng. In some embodiments, the threshold is 5 ng. In some embodiments, the threshold is 4 ng, 3 ng, 2 ng, or 1 ng.

### Amino Acid Substitutions and Modifications

RNA polymerase variants include at least one amino acid substitution, preferably at least two amino acid substitutions, relative to the wild type (WT) RNA polymerase. For example, with reference to WT T7 RNA polymerase having an amino acid sequence of SEQ ID NO:1, the glutamic acid (E) at position 350 is considered a “wild-type amino acid,” whereas a substitution of the glutamic acid for tryptophan at position 350 is considered an “amino acid substitution.” In some embodiments, the RNA polymerase variant is a T7 RNA polymerase variant comprising at least one (one or more) amino acid substitution relative to WT RNA polymerase (e.g., WT T7 RNA polymerase having an amino acid sequence of SEQ ID NO:1).

In some embodiments, RNA T7 polymerase variants comprise at least two amino acid substitutions. In some embodiments, an RNA T7 polymerase variant comprises at least three amino acid substitutions. In some embodiments, an RNA T7 polymerase variant comprises at least four amino acid substitutions. In some embodiments, an RNA T7 polymerase variant comprises at least five amino acid substitutions.

In some embodiments, an RNA polymerase variant comprises an amino acid sequence that includes an (at least one) amino acid modification that causes a loop structure of the RNA polymerase variant to undergo a conformational change to a helix structure as the RNA polymerase variant transitions from an initiation complex to an elongation complex. The amino acid substitution, in some embodiments, is a high propensity amino acid substitution. Examples of high-helix propensity amino acids include alanine, isoleucine, leucine, arginine, methionine, lysine, glutamine, and/or glutamate.

In some embodiments, an RNA polymerase variant comprises an amino acid sequence that includes an (at least one) amino acid substitution to introduce a polar, neutral amino acid. In some embodiments, a polar, neutral amino acid is selected from asparagine (N), cysteine (C), glutamine (Q), methionine (M), serine (S), and threonine (T). In some embodiments, an RNA polymerase variant comprises an amino acid sequence that includes an (at least one) amino acid substitution to introduce an aromatic amino acid. In some embodiments, an aromatic amino acid is selected from tryptophan (W), tyrosine (Y), and phenylalanine (F). In some embodiments, an RNA polymerase variant comprises an amino acid sequence that includes an (at least one) amino acid substitution to introduce a non-polar, aliphatic amino acid. In some embodiments, a non-polar, aliphatic amino acid is selected from alanine (A), glycine (G), isoleucine (I), leucine (L), proline (P), and valine (V). In some embodiments, an RNA polymerase variant comprises an amino acid sequence that includes an (at least one) amino acid substitution to introduce a positively charged amino acid. In some embodiments, a positively charged amino acid is selected from lysine (K), arginine (R), and histidine (H). In some embodiments, an RNA polymerase variant comprises an amino acid sequence that includes an (at least one) amino acid substitution to introduce a negatively charged amino acid. In some embodiments, a negatively charged amino acid is selected from aspartic acid (D) and glutamic acid (E).

In some embodiments, an RNA polymerase variant comprises an amino acid sequence that includes an (at least one) amino acid modification at a position that is not a conserved amino acid residue. Conserved amino acid residues are amino acids or amino acid types (e.g., individual amino acids such as Gly or Ser, or groups of amino acids that share similar properties such as amino acids with acidic functional groups) that are generally shared across multiple homologous sequences of the same protein. Conserved amino acid residues can be identified using sequence alignments of homologous amino acid sequences. A sequence alignment of approximately 1000 RNA polymerase sequences obtained using a Basic Local Alignment search allowed for a determination of the 240 positions of SEQ ID NO: 1 that are most likely to be conserved across RNA polymerase sequences. These 240 positions of SEQ ID NO: 1 that are most likely to be conserved across RNA polymerase sequences are at positions 5-6, 39, 269-277, 279, 281-282, 323-333, 411-448, 454-470, 472-474, 497-516, 532-560, 562-573, 626-646, 691, 693-702, 724-738, 775-794, 805-820, 828-833, 865-867, and 877-879. Accordingly, in some embodiments, an RNA polymerase variant comprises an RNA polymerase that includes an (at least one) amino acid modification at a position that is not one of positions 5-6, 39, 269-277, 279, 281-282, 323-333, 411-448, 454-470, 472-474, 497-516, 532-560, 562-573, 626-646, 691, 693-702, 724-738, 775-794, 805-820, 828-833, 865-867, and 877-879 of SEQ ID NO: 1. In some embodiments, an RNA polymerase variant may further comprise any number of amino acid modifications at any number of positions that are not one of positions 5-6, 39, 269-277, 279, 281-282, 323-333, 411-448, 454-470, 472-474, 497-516, 532-560, 562-573, 626-646, 691, 693-702, 724-738, 775-794, 805-820, 828-833, 865-867, and 877-879 of SEQ ID NO: 1. In some embodiments, an RNA polymerase variant comprising an amino acid sequence of any one of SEQ ID NO: 2-9 may further comprise an (at least one) additional amino acid modification at a position that is not one of positions 5-6, 39, 269-277, 279, 281-282, 323-333, 411-448, 454-470, 472-474, 497-516, 532-560, 562-573, 626-646, 691, 693-702, 724-738, 775-794, 805-820, 828-833, 865-867, and 877-879. Conversely, the amino acid positions that are not conserved are most likely to be modified or mutated. Accordingly, in some embodiments, an RNA polymerase variant comprises an RNA polymerase that includes an (at least one) amino acid modification at positions 1-4, 7-38, 40-268, 278, 280, 283-322, 334-410, 449-453, 471, 475-496, 517-531, 561, 574-625, 647-690, 692, 703-723, 739-774, 795-804, 821-827, 834-864, 868-876, and 880-883. In some embodiments, an RNA polymerase variant comprising an amino acid sequence of any one of SEQ ID NO: 2-9 may further comprise an (at least one) additional amino acid modification at positions 1-4, 7-38, 40-268, 278, 280, 283-322, 334-410, 449-453, 471, 475-496, 517-531, 561, 574-625, 647-690, 692, 703-723, 739-774, 795-804, 821-827, 834-864, 868-876, and 880-883.

In some embodiments, an RNA polymerase variant comprising an amino acid sequence of any one of SEQ ID NO: 2-9 may further comprise an (at least one) amino acid modification at any amino acid position that does not disrupt the secondary or tertiary structure of the RNA polymerase protein. In some embodiments, an RNA polymerase variant comprising amino acid sequence of any one of SEQ ID NO: 2-9 may further comprise an (at least one) amino acid modification at any amino acid position that does not disrupt the ability of the RNA polymerase protein to fold. In some embodiments, an RNA polymerase variant comprising an amino acid sequence of any one of SEQ ID NO: 2-9 may further comprise an (at least one) amino acid modification at any amino acid position that does not disrupt the ability of the RNA polymerase protein to bind to nucleic acids (e.g., DNA).

In some embodiments, an RNA polymerase variant comprises an amino acid sequence that includes an amino acid substitution at position 437 (e.g., N437Y), an amino acid substitution at position 387 (e.g., K387S), an amino acid substitution at position 350 (e.g., E350W), and an amino acid substitution at position 351 (e.g., D351V), relative to wild-type RNA polymerase, wherein the wild-type RNA polymerase comprises the amino acid sequence of SEQ ID NO: 1. In some embodiments, an RNA polymerase variant comprises N437Y, K387S, E350W, and D351V substitutions, relative to wild-type RNA polymerase, wherein the wild-type RNA polymerase comprises the amino acid sequence of SEQ ID NO: 1.

In some embodiments, an RNA polymerase variant comprises an amino acid sequence that includes an amino acid substitution at position 437 (e.g., N437Y), an amino acid substitution at position (e.g., E350W), and an amino acid substitution at position 351 (e.g., D351V), relative to wild-type RNA polymerase, wherein the wild-type RNA polymerase comprises the amino acid sequence of SEQ ID NO: 1. In some embodiments, an RNA polymerase variant comprises N437Y, E350W, and D351V substitutions, relative to wild-type RNA polymerase, wherein the wild-type RNA polymerase comprises the amino acid sequence of SEQ ID NO: 1.

In some embodiments, an RNA polymerase variant comprises an amino acid sequence that includes an amino acid substitution at position 387 (e.g., K387S), an amino acid substitution at position (e.g., E350W), and an amino acid substitution at position 351 (e.g., D351V) relative to wild-type RNA polymerase, wherein the wild-type RNA polymerase comprises the amino acid sequence of SEQ ID NO: 1. In some embodiments, an RNA polymerase variant comprises K387S, E350W, and D351V substitutions, relative to wild-type RNA polymerase, wherein the wild-type RNA polymerase comprises the amino acid sequence of SEQ ID NO: 1.

In some embodiments, an RNA polymerase variant comprises an amino acid sequence that includes an amino acid substitution at position 653 (e.g., D653W), an amino acid substitution at position 350 (e.g., E350W), and an amino acid substitution at position 351 (e.g., D351V) relative to wild-type RNA polymerase, wherein the wild-type RNA polymerase comprises the amino acid sequence of SEQ ID NO: 1. In some embodiments, an RNA polymerase variant comprises D653W, E350W, and D351V substitutions, relative to wild-type RNA polymerase, wherein the wild-type RNA polymerase comprises the amino acid sequence of SEQ ID NO: 1.

In some embodiments, an amino acid substitution at position K387 is a polar, neutral amino acid. In some embodiments, the polar, neutral amino acid is selected from asparagine (N), cysteine (C), glutamine (Q), methionine (M), serine (S), and threonine (T). Thus, in some embodiments, an amino acid substitution at position K387 is K387N, K387C, K387Q, K387M, K387S, or K387T.

In some embodiments, an amino acid substitution at position N437 is an aromatic amino acid. In some embodiments, the aromatic amino acid is selected from tryptophan (W), tyrosine (Y), and phenylalanine (F). Thus, in some embodiments, an amino acid substitution at position N437 is N437W, N437Y, or N437F.

In some embodiments, an amino acid substitution at position D653 is an aromatic amino acid. In some embodiments, the aromatic amino acid is selected from tryptophan (W), tyrosine (Y), and phenylalanine (F). Thus, in some embodiments, an amino acid substitution at position D653 is D653W, D653Y, or D653F.

In some embodiments, an amino acid substitution at position E350 is an aromatic amino acid. In some embodiments, the aromatic amino acid is selected from tryptophan (W), tyrosine (Y), and phenylalanine (F). Thus, in some embodiments, an amino acid substitution at position E350 is E350W, E350Y, or E350F.

In some embodiments, an amino acid substitution at position D351 is a non-polar, aliphatic amino acid. In some embodiments, the non-polar, aliphatic amino acid is selected from alanine (A), glycine (G), isoleucine (I), leucine (L), proline (P), and valine (V). Thus, in some embodiments, an amino acid substitution at position D351 is D351A, D351G, D351I, D351L, D351P, or D351V.

In some embodiments, an RNA polymerase variant comprises an amino acid sequence that includes an amino acid substitution at position R379 (e.g., R379A), relative to wild-type RNA polymerase, wherein the wild-type RNA polymerase comprises the amino acid sequence of SEQ ID NO: 1. In some embodiments, an amino acid substitution at position R379 is a non-polar, aliphatic amino acid; polar, neutral amino acid; aromatic amino acid; or charged amino acid amino acid. In some embodiments, the non-polar, aliphatic amino acid is selected from alanine (A), glycine (G), isoleucine (I), leucine (L), proline (P), and valine (V). In some embodiments, the aromatic amino acid is selected from tryptophan (W), tyrosine (Y), and phenylalanine (F). In some embodiments, the charged amino acid is a positively charged amino acid (e.g., lysine (K) or histidine (H)) or a negatively charged amino acid (e.g., glutamic acid (E) or aspartic acid (D)). In some embodiments, an amino acid substitution at position R379 is R379A, R379K, R379E, or R379W.

In some embodiments, an RNA polymerase variant comprises an amino acid sequence that includes an amino acid substitution at position Y385 (e.g., Y385A), relative to wild-type RNA polymerase, wherein the wild-type RNA polymerase comprises the amino acid sequence of SEQ ID NO: 1. In some embodiments, an amino acid substitution at position Y385 is a non-polar, aliphatic amino acid; polar, neutral amino acid; aromatic amino acid; or charged amino acid amino acid. In some embodiments, the non-polar, aliphatic amino acid is selected from alanine (A), glycine (G), isoleucine (I), leucine (L), proline (P), and valine (V). In some embodiments, the aromatic amino acid is selected from tryptophan (W) and phenylalanine (F). In some embodiments, the charged amino acid is a positively charged amino acid (e.g., lysine (K), histidine (H), or arginine (R)) or a negatively charged amino acid (e.g., glutamic acid (E) or aspartic acid (D)). In some embodiments, an amino acid substitution at position Y385 is Y385A, Y385K, Y385W, or Y385V.

In some embodiments, an RNA polymerase variant comprises an amino acid sequence that includes an amino acid substitution at position R386 (e.g., R386A), relative to wild-type RNA polymerase, wherein the wild-type RNA polymerase comprises the amino acid sequence of SEQ ID NO: 1. In some embodiments, an amino acid substitution at position R386 is a non-polar, aliphatic amino acid; polar, neutral amino acid; aromatic amino acid; or charged amino acid amino acid. In some embodiments, the non-polar, aliphatic amino acid is selected from alanine (A), glycine (G), isoleucine (I), leucine (L), proline (P), and valine (V). In some embodiments, the charged amino acid is a positively charged amino acid (e.g., lysine (K) or histidine (H)) or a negatively charged amino acid (e.g., glutamic acid (E) or aspartic acid (D)). In some embodiments, the aromatic amino acid is selected from tryptophan (W), tyrosine (Y), and phenylalanine (F). In some embodiments, an amino acid substitution at position R386 is R386A, R386K, or R386Y.

In some embodiments, an RNA polymerase variant comprises an amino acid sequence that includes an amino acid substitution at position D388 (e.g., D388A), relative to wild-type RNA polymerase, wherein the wild-type RNA polymerase comprises the amino acid sequence of SEQ ID NO: 1. In some embodiments, an amino acid substitution at position D388 is a non-polar, aliphatic amino acid; polar, neutral amino acid; aromatic amino acid; or charged amino acid amino acid. In some embodiments, the non-polar, aliphatic amino acid is selected from alanine (A), glycine (G), isoleucine (I), leucine (L), proline (P), and valine (V). In some embodiments, the polar, neutral amino acid is selected from asparagine (N), cysteine (C), glutamine (Q), methionine (M), serine (S), and threonine (T). In some embodiments, the aromatic amino acid is selected from tryptophan (W), tyrosine (Y), and phenylalanine (F). In some embodiments, an amino acid substitution at position D388 is D388A, D388N, or D388Y.

In some embodiments, an RNA polymerase variant comprises an amino acid sequence that includes an amino acid substitution at position K389 (e.g., K389A), relative to wild-type RNA polymerase, wherein the wild-type RNA polymerase comprises the amino acid sequence of SEQ ID NO: 1. In some embodiments, an amino acid substitution at position K389 is a non-polar, aliphatic amino acid; polar, neutral amino acid; aromatic amino acid; or charged amino acid amino acid. In some embodiments, the non-polar, aliphatic amino acid is selected from alanine (A), glycine (G), isoleucine (I), leucine (L), proline (P), and valine (V). In some embodiments, the polar, neutral amino acid is selected from asparagine (N), cysteine (C), glutamine (Q), methionine (M), serine (S), and threonine (T). In some embodiments, the aromatic amino acid is selected from tryptophan (W), tyrosine (Y), and phenylalanine (F). In some embodiments, an amino acid substitution at position K389 is K389A, K389S, or K389Y.

In some embodiments, an RNA polymerase variant comprises an amino acid sequence that includes an amino acid substitution at position R391 (e.g., R391A), relative to wild-type RNA polymerase, wherein the wild-type RNA polymerase comprises the amino acid sequence of SEQ ID NO: 1. In some embodiments, an amino acid substitution at position R391 is a non-polar, aliphatic amino acid; polar, neutral amino acid; aromatic amino acid; or charged amino acid amino acid. In some embodiments, the non-polar, aliphatic amino acid is selected from alanine (A), glycine (G), isoleucine (I), leucine (L), proline (P), and valine (V). In some embodiments, an amino acid substitution at position R391 is R391A.

In some embodiments, an RNA polymerase variant comprises an amino acid sequence that includes an amino acid substitution at position R394 (e.g., R394A), relative to wild-type RNA polymerase, wherein the wild-type RNA polymerase comprises the amino acid sequence of SEQ ID NO: 1. In some embodiments, an amino acid substitution at position R394 is a non-polar, aliphatic amino acid; polar, neutral amino acid; aromatic amino acid; or charged amino acid amino acid. In some embodiments, the non-polar, aliphatic amino acid is selected from alanine (A), glycine (G), isoleucine (I), leucine (L), proline (P), and valine (V). In some embodiments, the polar, neutral amino acid is selected from asparagine (N), cysteine (C), glutamine (Q), methionine (M), serine (S), and threonine (T). In some embodiments, the aromatic amino acid is selected from tryptophan (W), tyrosine (Y), and phenylalanine (F). In some embodiments, an amino acid substitution at position R394 is R394A, R394Q, or R394Y.

In some embodiments, an RNA polymerase variant comprises an amino acid sequence that includes an amino acid substitution at position R395 (e.g., R395A), relative to wild-type RNA polymerase, wherein the wild-type RNA polymerase comprises the amino acid sequence of SEQ ID NO: 1. In some embodiments, an amino acid substitution at position R395 is a non-polar, aliphatic amino acid; polar, neutral amino acid; aromatic amino acid; or charged amino acid amino acid. In some embodiments, the non-polar, aliphatic amino acid is selected from alanine (A), glycine (G), isoleucine (I), leucine (L), proline (P), and valine (V). In some embodiments, an amino acid substitution at position R395 is R395A.

In some embodiments, an RNA polymerase variant comprises an amino acid sequence that includes an amino acid substitution at position D471 (e.g., D471A), relative to wild-type RNA polymerase, wherein the wild-type RNA polymerase comprises the amino acid sequence of SEQ ID NO: 1. In some embodiments, an amino acid substitution at position D471 is a non-polar, aliphatic amino acid; polar, neutral amino acid; aromatic amino acid; or charged amino acid amino acid. In some embodiments, the non-polar, aliphatic amino acid is selected from alanine (A), glycine (G), isoleucine (I), leucine (L), proline (P), and valine (V). In some embodiments, the charged amino acid is a positively charged amino acid (e.g., lysine (K), histidine (H), or arginine (R)) or a negatively charged amino acid (e.g., glutamic acid). In some embodiments, the aromatic amino acid is selected from tryptophan (W), tyrosine (Y), and phenylalanine (F). In some embodiments, an amino acid substitution at position D471 is D471A, D471E, or D471Y.

In some embodiments, an RNA polymerase variant comprises an amino acid sequence that includes an amino acid substitution at position R627 (e.g., R627A), relative to wild-type RNA polymerase, wherein the wild-type RNA polymerase comprises the amino acid sequence of SEQ ID NO: 1. In some embodiments, an amino acid substitution at position R627 is a non-polar, aliphatic amino acid; polar, neutral amino acid; aromatic amino acid; or charged amino acid amino acid. In some embodiments, the non-polar, aliphatic amino acid is selected from alanine (A), glycine (G), isoleucine (I), leucine (L), proline (P), and valine (V). In some embodiments, an amino acid substitution at position R627 is R627A.

In some embodiments, an RNA polymerase variant comprises an amino acid sequence that includes an amino acid substitution at position R631 (e.g., R631A), relative to wild-type RNA polymerase, wherein the wild-type RNA polymerase comprises the amino acid sequence of SEQ ID NO: 1. In some embodiments, an amino acid substitution at position R631 is a non-polar, aliphatic amino acid; polar, neutral amino acid; aromatic amino acid; or charged amino acid amino acid. In some embodiments, the non-polar, aliphatic amino acid is selected from alanine (A), glycine (G), isoleucine (I), leucine (L), proline (P), and valine (V). In some embodiments, an amino acid substitution at position R631 is R631A.

In some embodiments, an RNA polymerase variant comprises an amino acid sequence that includes an amino acid substitution at position R632 (e.g., R632A), relative to wild-type RNA polymerase, wherein the wild-type RNA polymerase comprises the amino acid sequence of SEQ ID NO: 1. In some embodiments, an amino acid substitution at position R632 is a non-polar, aliphatic amino acid; polar, neutral amino acid; aromatic amino acid; or charged amino acid amino acid. In some embodiments, the non-polar, aliphatic amino acid is selected from alanine (A), glycine (G), isoleucine (I), leucine (L), proline (P), and valine (V). In some embodiments, the aromatic amino acid is selected from tryptophan (W), tyrosine (Y), and phenylalanine (F). In some embodiments, the polar, neutral amino acid is selected from asparagine (N), cysteine (C), glutamine (Q), methionine (M), serine (S), and threonine (T). In some embodiments, an amino acid substitution at position R632 is R632D, R632A, R632Q, or R632Y.

In some embodiments, an RNA polymerase variant comprises an amino acid sequence that includes an amino acid substitution at position G640 (e.g., G640A), relative to wild-type RNA polymerase, wherein the wild-type RNA polymerase comprises the amino acid sequence of SEQ ID NO: 1. In some embodiments, an amino acid substitution at position G640 is a non-polar, aliphatic amino acid; polar, neutral amino acid; aromatic amino acid; or charged amino acid amino acid. In some embodiments, the non-polar, aliphatic amino acid is selected from alanine (A), glycine (G), isoleucine (I), leucine (L), proline (P), and valine (V). In some embodiments, an amino acid substitution at position G640 is G640A.

In some embodiments, an RNA polymerase variant comprises an amino acid sequence that includes an amino acid substitution at position G645 (e.g., G645A), relative to wild-type RNA polymerase, wherein the wild-type RNA polymerase comprises the amino acid sequence of SEQ ID NO: 1. In some embodiments, an amino acid substitution at position G645 is a non-polar, aliphatic amino acid; polar, neutral amino acid; aromatic amino acid; or charged amino acid amino acid. In some embodiments, the non-polar, aliphatic amino acid is selected from alanine (A), glycine (G), isoleucine (I), leucine (L), proline (P), and valine (V). In some embodiments, an amino acid substitution at position G645 is G645A.

In some embodiments, an RNA polymerase variant comprises an amino acid sequence that includes an amino acid substitution at position Q648 (e.g., Q648A), relative to wild-type RNA polymerase, wherein the wild-type RNA polymerase comprises the amino acid sequence of SEQ ID NO: 1. In some embodiments, an amino acid substitution at position Q648 is a non-polar, aliphatic amino acid; polar, neutral amino acid; aromatic amino acid; or charged amino acid amino acid. In some embodiments, the non-polar, aliphatic amino acid is selected from alanine (A), glycine (G), isoleucine (I), leucine (L), proline (P), and valine (V). In some embodiments, the aromatic amino acid is selected from tryptophan (W), tyrosine (Y), and phenylalanine (F). In some embodiments, the charged amino acid is a positively charged amino acid (e.g., lysine (K), histidine (H), or arginine (R)) or a negatively charged amino acid (e.g., glutamic acid (E) or aspartic acid (D)). In some embodiments, an amino acid substitution at position Q648 is Q648A, Q648R, Q648D, Q648E, or Q648Y.

In some embodiments, an RNA polymerase variant comprises an amino acid sequence that includes an amino acid substitution at position Q649 (e.g., Q649A), relative to wild-type RNA polymerase, wherein the wild-type RNA polymerase comprises the amino acid sequence of SEQ ID NO: 1. In some embodiments, an amino acid substitution at position Q649 is a non-polar, aliphatic amino acid; polar, neutral amino acid; aromatic amino acid; or charged amino acid amino acid. In some embodiments, the non-polar, aliphatic amino acid is selected from alanine (A), glycine (G), isoleucine (I), leucine (L), proline (P), and valine (V). In some embodiments, an amino acid substitution at position Q649 is Q649A.

In some embodiments, an RNA polymerase variant comprises an amino acid sequence that includes an amino acid substitution at position E652 (e.g., E652A), relative to wild-type RNA polymerase, wherein the wild-type RNA polymerase comprises the amino acid sequence of SEQ ID NO: 1. In some embodiments, an amino acid substitution at position E652 is a non-polar, aliphatic amino acid; polar, neutral amino acid; aromatic amino acid; or charged amino acid amino acid. In some embodiments, the non-polar, aliphatic amino acid is selected from alanine (A), glycine (G), isoleucine (I), leucine (L), proline (P), and valine (V). In some embodiments, the aromatic amino acid is selected from tryptophan (W), tyrosine (Y), and phenylalanine (F). In some embodiments, the charged amino acid is a positively charged amino acid (e.g., lysine (K), histidine (H), or arginine (R)). In some embodiments, an amino acid substitution at position E652 is E652R, E652A, or E652Y.

In some embodiments, an RNA polymerase variant comprises an amino acid sequence that includes an amino acid substitution at position D653 (e.g., D653A), relative to wild-type RNA polymerase, wherein the wild-type RNA polymerase comprises the amino acid sequence of SEQ ID NO: 1. In some embodiments, an amino acid substitution at position D653 is a non-polar, aliphatic amino acid; polar, neutral amino acid; aromatic amino acid; or charged amino acid amino acid. In some embodiments, the non-polar, aliphatic amino acid is selected from alanine (A), glycine (G), isoleucine (I), leucine (L), proline (P), and valine (V). In some embodiments, the aromatic amino acid is selected from tryptophan (W), tyrosine (Y), and phenylalanine (F). In some embodiments, the charged amino acid is a positively charged amino acid (e.g., lysine (K), histidine (H), or arginine (R)). In some embodiments, an amino acid substitution at position D653 is D653K, D653A, or D653Y.

In some embodiments, an RNA polymerase variant comprises an amino acid sequence that includes an amino acid substitution at position Q656 (e.g., Q656A), relative to wild-type RNA polymerase, wherein the wild-type RNA polymerase comprises the amino acid sequence of SEQ ID NO: 1. In some embodiments, an amino acid substitution at position Q656 is a non-polar, aliphatic amino acid; polar, neutral amino acid; aromatic amino acid; or charged amino acid amino acid. In some embodiments, the non-polar, aliphatic amino acid is selected from alanine (A), glycine (G), isoleucine (I), leucine (L), proline (P), and valine (V). In some embodiments, the aromatic amino acid is selected from tryptophan (W), tyrosine (Y), and phenylalanine (F). In some embodiments, the charged amino acid is a positively charged amino acid (e.g., lysine (K), histidine (H), or arginine (R)) or a negatively charged amino acid (e.g., glutamic acid (E) or aspartic acid (D)). In some embodiments, an amino acid substitution at position Q656 is Q656K, Q656A, Q656E, or Q656Y.

In some embodiments, an RNA polymerase variant comprises an amino acid sequence that includes an amino acid substitution at position P657 (e.g., P657A), relative to wild-type RNA polymerase, wherein the wild-type RNA polymerase comprises the amino acid sequence of SEQ ID NO: 1. In some embodiments, an amino acid substitution at position P657 is a non-polar, aliphatic amino acid; polar, neutral amino acid; aromatic amino acid; or charged amino acid amino acid. In some embodiments, the non-polar, aliphatic amino acid is selected from alanine (A), glycine (G), isoleucine (I), leucine (L), proline (P), and valine (V). In some embodiments, the aromatic amino acid is selected from tryptophan (W), tyrosine (Y), and phenylalanine (F). In some embodiments, the charged amino acid is a positively charged amino acid (e.g., lysine (K), histidine (H), or arginine (R)) or a negatively charged amino acid (e.g., glutamic acid (E) or aspartic acid (D)). In some embodiments, an amino acid substitution at position P657 is P657G, P657A, P657E, or P657Y.

TABLE 1

| RNA | | |
|---|---|---|
| Polymerase | | SEQ ID |
| Variants | Amino Acid Sequence | NO |
| E350X¹ | MNTINIAKNDFSDIELAAIPFNTLADHYGERLAREQLALEHESYEMGEARFRK | 2 |
| D351X² | MFERQLKAGEVADNAAAKPLITTLLPKMIARINDWFEEVKAKRGKRPTAFQFL | |
| K387X³ | QEIKPEAVAYITIKTTLACLTSADNTTVQAVASAIGRAIEDEARFGRIRDLEA | |
| N437X⁴ | KHFKKNVEEQLNKRVGHVYKKAFMQVVEADMLSKGLLGGEAWSSWHKEDSIHV | |
| | GVRCIEMLIESTGMVSLHRQNAGVVGQDSETIELAPEYAEAIATRAGALAGIS | |
| | PMFQPCVVPPKPWTGITGGGYWANGRRPLALVRTHSKKALMRYEDVYMPEVYK | |
| | AINIAQNTAWKINKKVLAVANVITKWKHCPVX¹X²IPAIEREELPMKPEDIDM | |
| | NPEALTAWKRAAAAVYRX³DKARKSRRISLEFMLEQANKFANHKAIWFPYNMD | |
| | WRGRVYAVSMFNPQGX⁴DMTKGLLTLAKGKPIGKEGYYWLKIHGANCAGVDKV | |
| | PFPERIKFIEENHENIMACAKSPLENTWWAEQDSPFCFLAFCFEYAGVQHHGL | |
| | SYNCSLPLAFDGSCSGIQHFSAMLRDEVGGRAVNLLPSETVQDIYGIVAKKVN | |
| | EILQADAINGTDNEVVTVTDENTGEISEKVKLGTKALAGQWLAYGVTRSVTKR | |
| | SVMTLAYGSKEFGFRQQVLEDTIQPAIDSGKGLMFTQPNQAAGYMAKLIWESV | |
| | SVTVVAAVEAMNWLKSAAKLLAAEVKDKKTGEILRKRCAVHWVTPDGFPVWQE | |
| | YKKPIQTRLNLMFLGQFRLQPTINTNKDSEIDAHKQESGIAPNFVHSQDGSHL | |
| | RKTVVWAHEKYGIESFALIHDSFGTIPADAANLFKAVRETMVDTYESCDVLAD | |
| | FYDQFADQLHESQLDKMPALPAKGNLNLRDILESDFAFA, wherein X¹ is an | |
| | aromatic amino acid, optionally selected from W, Y, and F; X² is | |
| | selected from a non-polar, aliphatic amino acid, optionally selected | |
| | from A, G, I, L, P, and V; X³ is a polar, neutral amino acid, optionally | |
| | selected from N, C, Q, M, S, and T; and X⁴ is an aromatic amino acid, | |
| | optionally selected from W, Y, and F | |
| E350X¹ | MNTINIAKNDFSDIELAAIPFNTLADHYGERLAREQLALEHESYEMGEARFRK | 3 |
| D351X² | MFERQLKAGEVADNAAAKPLITTLLPKMIARINDWFEEVKAKRGKRPTAFQFL | |
| N437X⁴ | QEIKPEAVAYITIKTTLACLTSADNTTVQAVASAIGRAIEDEARFGRIRDLEA | |
| | KHFKKNVEEQLNKRVGHVYKKAFMQVVEADMLSKGLLGGEAWSSWHKEDSIHV | |
| | GVRCIEMLIESTGMVSLHRQNAGVVGODSETIELAPEYAEAIATRAGALAGIS | |
| | PMFQPCVVPPKPWTGITGGGYWANGRRPLALVRTHSKKALMRYEDVYMPEVYK | |
| | AINIAQNTAWKINKKVLAVANVITKWKHCPVX¹X²IPAIEREELPMKPEDIDM | |
| | NPEALTAWKRAAAAVYRKDKARKSRRISLEFMLEQANKFANHKAIWFPYNMDW | |
| | RGRVYAVSMFNPQGX⁴DMTKGLLTLAKGKPIGKEGYYWLKIHGANCAGVDKVP | |
| | FPERIKFIEENHENIMACAKSPLENTWWAEQDSPFCFLAFCFEYAGVQHHGLS | |
| | YNCSLPLAFDGSCSGIQHFSAMLRDEVGGRAVNLLPSETVQDIYGIVAKKVNE | |
| | ILQADAINGTDNEVVTVTDENTGEISEKVKLGTKALAGQWLAYGVTRSVTKRS | |
| | VMTLAYGSKEFGFRQQVLEDTIQPAIDSGKGLMFTQPNQAAGYMAKLIWESVS | |
| | VTVVAAVEAMNWLKSAAKLLAAEVKDKKTGEILRKRCAVHWVTPDGFPVWQEY | |
| | KKPIQTRLNLMFLGQFRLQPTINTNKDSEIDAHKQESGIAPNFVHSQDGSHLR | |
| | KTVVWAHEKYGIESFALIHDSFGTIPADAANLFKAVRETMVDTYESCDVLADF | |
| | YDQFADQLHESQLDKMPALPAKGNLNLRDILESDFAFA, wherein X¹ is an | |
| | aromatic amino acid, optionally selected from W, Y, and F; X² is | |
| | selected from a non-polar, aliphatic amino acid, optionally selected | |
| | from A, G, I, L, P, and V; and X⁴ is an aromatic amino acid, optionally | |
| | selected from W, Y, and F | |
| E350X¹ | MNTINIAKNDFSDIELAAIPFNTLADHYGERLAREQLALEHESYEMGEARFRK | 4 |
| D351X² | MFERQLKAGEVADNAAAKPLITTLLPKMIARINDWFEEVKAKRGKRPTAFQFL | |
| K387X³ | QEIKPEAVAYITIKTTLACLTSADNTTVQAVASAIGRAIEDEARFGRIRDLEA | |
| | KHFKKNVEEQLNKRVGHVYKKAFMQVVEADMLSKGLLGGEAWSSWHKEDSIHV | |
| | GVRCIEMLIESTGMVSLHRQNAGVVGQDSETIELAPEYAEAIATRAGALAGIS | |
| | PMFQPCVVPPKPWTGITGGGYWANGRRPLALVRTHSKKALMRYEDVYMPEVYK | |
| | AINIAQNTAWKINKKVLAVANVITKWKHCPVX¹X²IPAIEREELPMKPEDIDM | |
| | NPEALTAWKRAAAAVYRX³DKARKSRRISLEFMLEQANKFANHKAIWFPYNMD | |
| | WRGRVYAVSMFNPQGNDMTKGLLTLAKGKPIGKEGYYWLKIHGANCAGVDKVP | |
| | FPERIKFIEENHENIMACAKSPLENTWWAEQDSPFCFLAFCFEYAGVQHHGLS | |
| | YNCSLPLAFDGSCSGIQHFSAMLRDEVGGRAVNLLPSETVQDIYGIVAKKVNE | |
| | ILQADAINGTDNEVVTVTDENTGEISEKVKLGTKALAGQWLAYGVTRSVTKRS | |
| | VMTLAYGSKEFGFRQQVLEDTIQPAIDSGKGLMFTQPNQAAGYMAKLIWESVS | |
| | VTVVAAVEAMNWLKSAAKLLAAEVKDKKTGEILRKRCAVHWVTPDGFPVWQEY | |
| | KKPIQTRLNLMFLGQFRLQPTINTNKDSEIDAHKQESGIAPNFVHSQDGSHLR | |
| | KTVVWAHEKYGIESFALIHDSFGTIPADAANLFKAVRETMVDTYESCDVLADE | |
| | YDQFADQLHESQLDKMPALPAKGNLNLRDILESDFAFA, wherein X¹ is an | |
| | aromatic amino acid, optionally selected from W, Y, and F; X² is | |
| | selected from a non-polar, aliphatic amino acid, optionally selected | |
| | from A, G, I, L, P, and V; and X³ is a polar, neutral amino acid, | |
| | optionally selected from N, C, Q, M, S, and T | |
| E350X¹ | MNTINIAKNDFSDIELAAIPFNTLADHYGERLAREQLALEHESYEMGEARFRK | 5 |
| D351X² | MFERQLKAGEVADNAAAKPLITTLLPKMIARINDWFEEVKAKRGKRPTAFQFL | |
| D653X⁵ | QEIKPEAVAYITIKTTLACLTSADNTTVQAVASAIGRAIEDEARFGRIRDLEA | |
| | KHFKKNVEEQLNKRVGHVYKKAFMQVVEADMLSKGLLGGEAWSSWHKEDSIHV | |
| | GVRCIEMLIESTGMVSLHRQNAGVVGQDSETIELAPEYAEAIATRAGALAGIS | |
| | PMFQPCVVPPKPWTGITGGGYWANGRRPLALVRTHSKKALMRYEDVYMPEVYK | |
| | AINIAQNTAWKINKKVLAVANVITKWKHCPVX¹X²IPAIEREELPMKPEDIDM | |
| | NPEALTAWKRAAAAVYRKDKARKSRRISLEFMLEQANKFANHKAIWFPYNMDW | |
| | RGRVYAVSMFNPQGNDMTKGLLTLAKGKPIGKEGYYWLKIHGANCAGVDKVPF | |
| | PERIKFIEENHENIMACAKSPLENTWWAEQDSPFCFLAFCFEYAGVQHHGLSY | |
| | NCSLPLAFDGSCSGIQHFSAMLRDEVGGRAVNLLPSETVQDIYGIVAKKVNEI | |
| | LQADAINGTDNEVVTVTDENTGEISEKVKLGTKALAGQWLAYGVTRSVTKRSV | |
| | MTLAYGSKEFGFRQQVLEX⁵TIQPAIDSGKGLMFTQPNQAAGYMAKLIWESVS | |
| | VTVVAAVEAMNWLKSAAKLLAAEVKDKKTGEILRKRCAVHWVTPDGFPVWQEY | |
| | KKPIQTRLNLMFLGQFRLOPTINTNKDSEIDAHKQESGIAPNFVHSQDGSHLR | |
| | KTVVWAHEKYGIESFALIHDSFGTIPADAANLFKAVRETMVDTYESCDVLADE | |
| | YDQFADQLHESQLDKMPALPAKGNLNLRDILESDFAFA, wherein X¹ is an | |
| | aromatic amino acid, optionally selected from W, Y, and F; X² is | |
| | selected from a non-polar, aliphatic amino acid, optionally selected | |
| | from A, G, I, L, P, and V; and X⁵ is an aromatic amino acid, optionally | |
| | selected from W, Y, and F | |
| E350W | MNTINIAKNDFSDIELAAIPFNTLADHYGERLAREQLALEHESYEMGEARFRK | 6 |
| D351V | MFERQLKAGEVADNAAAKPLITTLLPKMIARINDWFEEVKAKRGKRPTAFQFL | |
| K387S | QEIKPEAVAYITIKTTLACLTSADNTTVQAVASAIGRAIEDEARFGRIRDLEA | |
| N437Y | KHFKKNVEEQLNKRVGHVYKKAFMQVVEADMLSKGLLGGEAWSSWHKEDSIHV | |
| | GVRCIEMLIESTGMVSLHRQNAGVVGODSETIELAPEYAEAIATRAGALAGIS | |
| | PMFQPCVVPPKPWTGITGGGYWANGRRPLALVRTHSKKALMRYEDVYMPEVYK | |
| | AINIAQNTAWKINKKVLAVANVITKWKHCPVWVIPAIEREELPMKPEDIDMNP | |
| | EALTAWKRAAAAVYRSDKARKSRRISLEFMLEQANKFANHKAIWFPYNMDWRG | |
| | RVYAVSMFNPQGYDMTKGLLTLAKGKPIGKEGYYWLKIHGANCAGVDKVPFPE | |
| | RIKFIEENHENIMACAKSPLENTWWAEQDSPFCFLAFCFEYAGVQHHGLSYNC | |
| | SLPLAFDGSCSGIQHFSAMLRDEVGGRAVNLLPSETVQDIYGIVAKKVNEILQ | |
| | ADAINGTDNEVVTVTDENTGEISEKVKLGTKALAGQWLAYGVTRSVTKRSVMT | |
| | LAYGSKEFGFRQQVLEDTIQPAIDSGKGLMFTQPNQAAGYMAKLIWESVSVTV | |
| | VAAVEAMNWLKSAAKLLAAEVKDKKTGEILRKRCAVHWVTPDGFPVWQEYKKP | |
| | IQTRLNLMFLGQFRLQPTINTNKDSEIDAHKQESGIAPNFVHSQDGSHLRKTV | |
| | VWAHEKYGIESFALIHDSFGTIPADAANLFKAVRETMVDTYESCDVLADFYDQ | |
| | FADQLHESQLDKMPALPAKGNLNLRDILESDFAFA | |
| E350W | MNTINIAKNDFSDIELAAIPFNTLADHYGERLAREQLALEHESYEMGEARFRK | 7 |
| D351V | MFERQLKAGEVADNAAAKPLITTLLPKMIARINDWFEEVKAKRGKRPTAFQFL | |
| N437Y | QEIKPEAVAYITIKTTLACLTSADNTTVQAVASAIGRAIEDEARFGRIRDLEA | |
| | KHFKKNVEEQLNKRVGHVYKKAFMQVVEADMLSKGLLGGEAWSSWHKEDSIHV | |
| | GVRCIEMLIESTGMVSLHRQNAGVVGODSETIELAPEYAEAIATRAGALAGIS | |
| | PMFQPCVVPPKPWTGITGGGYWANGRRPLALVRTHSKKALMRYEDVYMPEVYK | |
| | AINIAQNTAWKINKKVLAVANVITKWKHCPVWVIPAIEREELPMKPEDIDMNP | |
| | EALTAWKRAAAAVYRKDKARKSRRISLEFMLEQANKFANHKAIWFPYNMDWRG | |
| | RVYAVSMFNPQGYDMTKGLLTLAKGKPIGKEGYYWLKIHGANCAGVDKVPFPE | |
| | RIKFIEENHENIMACAKSPLENTWWAEQDSPFCFLAFCFEYAGVQHHGLSYNC | |
| | SLPLAFDGSCSGIQHFSAMLRDEVGGRAVNLLPSETVQDIYGIVAKKVNEILQ | |
| | ADAINGTDNEVVTVTDENTGEISEKVKLGTKALAGQWLAYGVTRSVTKRSVMT | |
| | LAYGSKEFGFRQQVLEDTIQPAIDSGKGLMFTQPNQAAGYMAKLIWESVSVTV | |
| | VAAVEAMNWLKSAAKLLAAEVKDKKTGEILRKRCAVHWVTPDGFPVWQEYKKP | |
| | IQTRLNLMFLGQFRLQPTINTNKDSEIDAHKQESGIAPNFVHSQDGSHLRKTV | |
| | VWAHEKYGIESFALIHDSFGTIPADAANLFKAVRETMVDTYESCDVLADFYDQ | |
| | FADQLHESQLDKMPALPAKGNLNLRDILESDFAFA | |
| E350W | MNTINIAKNDFSDIELAAIPFNTLADHYGERLAREQLALEHESYEMGEARFRK | 8 |
| D351V | MFERQLKAGEVADNAAAKPLITTLLPKMIARINDWFEEVKAKRGKRPTAFQFL | |
| K387S | QEIKPEAVAYITIKTTLACLTSADNTTVQAVASAIGRAIEDEARFGRIRDLEA | |
| | KHFKKNVEEQLNKRVGHVYKKAFMQVVEADMLSKGLLGGEAWSSWHKEDSIHV | |
| | GVRCIEMLIESTGMVSLHRQNAGVVGODSETIELAPEYAEAIATRAGALAGIS | |
| | PMFQPCVVPPKPWTGITGGGYWANGRRPLALVRTHSKKALMRYEDVYMPEVYK | |
| | AINIAQNTAWKINKKVLAVANVITKWKHCPVWVIPAIEREELPMKPEDIDMNP | |
| | EALTAWKRAAAAVYRSDKARKSRRISLEFMLEQANKFANHKAIWFPYNMDWRG | |
| | RVYAVSMFNPQGNDMTKGLLTLAKGKPIGKEGYYWLKIHGANCAGVDKVPFPE | |
| | RIKFIEENHENIMACAKSPLENTWWAEQDSPFCFLAFCFEYAGVQHHGLSYNC | |
| | SLPLAFDGSCSGIQHFSAMLRDEVGGRAVNLLPSETVQDIYGIVAKKVNEILQ | |
| | ADAINGTDNEVVTVTDENTGEISEKVKLGTKALAGQWLAYGVTRSVTKRSVMT | |
| | LAYGSKEFGFRQQVLEDTIQPAIDSGKGLMFTQPNQAAGYMAKLIWESVSVTV | |
| | VAAVEAMNWLKSAAKLLAAEVKDKKTGEILRKRCAVHWVTPDGFPVWQEYKKP | |
| | IQTRLNLMFLGQFRLQPTINTNKDSEIDAHKQESGIAPNFVHSQDGSHLRKTV | |
| | VWAHEKYGIESFALIHDSFGTIPADAANLFKAVRETMVDTYESCDVLADFYDQ | |
| | FADQLHESQLDKMPALPAKGNLNLRDILESDFAFA | |
| E350W | MNTINIAKNDFSDIELAAIPFNTLADHYGERLAREQLALEHESYEMGEARFRK | 9 |
| D351V | MFERQLKAGEVADNAAAKPLITTLLPKMIARINDWFEEVKAKRGKRPTAFQFL | |
| D653W | QEIKPEAVAYITIKTTLACLTSADNTTVQAVASAIGRAIEDEARFGRIRDLEA | |
| | KHFKKNVEEQLNKRVGHVYKKAFMQVVEADMLSKGLLGGEAWSSWHKEDSIHV | |
| | GVRCIEMLIESTGMVSLHRQNAGVVGODSETIELAPEYAEAIATRAGALAGIS | |
| | PMFQPCVVPPKPWTGITGGGYWANGRRPLALVRTHSKKALMRYEDVYMPEVYK | |
| | AINIAQNTAWKINKKVLAVANVITKWKHCPVWVIPAIEREELPMKPEDIDMNP | |
| | EALTAWKRAAAAVYRKDKARKSRRISLEFMLEQANKFANHKAIWFPYNMDWRG | |
| | RVYAVSMFNPQGNDMTKGLLTLAKGKPIGKEGYYWLKIHGANCAGVDKVPFPE | |
| | RIKFIEENHENIMACAKSPLENTWWAEQDSPFCFLAFCFEYAGVQHHGLSYNC | |
| | SLPLAFDGSCSGIQHFSAMLRDEVGGRAVNLLPSETVQDIYGIVAKKVNEILQ | |
| | ADAINGTDNEVVTVTDENTGEISEKVKLGTKALAGQWLAYGVTRSVTKRSVMT | |
| | LAYGSKEFGFRQQVLEWTIQPAIDSGKGLMFTQPNQAAGYMAKLIWESVSVTV | |
| | VAAVEAMNWLKSAAKLLAAEVKDKKTGEILRKRCAVHWVTPDGFPVWQEYKKP | |
| | IQTRLNLMFLGQFRLQPTINTNKDSEIDAHKQESGIAPNFVHSQDGSHLRKTV | |
| | VWAHEKYGIESFALIHDSFGTIPADAANLFKAVRETMVDTYESCDVLADFYDQ | |
| | FADQLHESQLDKMPALPAKGNLNLRDILESDFAFA | |

In some embodiments, an RNA polymerase variant further comprises one or more purification tags. For example, an RNA polymerase variant may comprise a histidine purification tag (e.g., an amino acid sequence of -HHHHHH- (SEQ ID NO: 14)) or any other sequence of amino acids useful for purification. A histidine purification tag or similarly charged amino acid sequence is capable of binding to Ni²⁺ resin. In some embodiments, a histidine purification tag comprises the amino acid sequence of -HHHHHHV- (SEQ ID NO: 15). In some embodiments, a purification tag is an N-terminal purification tag that is covalently attached to the N-terminus of an RNA polymerase variant. In some embodiments, a purification tag is a C-terminal purification tag that is covalently attached to the C-terminus of an RNA polymerase variant. In some embodiments, a protein purification tag is a FLAG tag (e.g., an amino acid sequence of -5 DYKDDDK- (SEQ ID NO: 16)) or a hemagglutinin tag. In some embodiments, an RNA polymerase variant comprising an N-terminal His tag comprises any one of SEQ ID NOs: 10-13.

TABLE 2
RNA Polymerase Variants comprising an N-terminal His tag

| RNA | | |
|---|---|---|
| Polymerase | | SEQ ID |
| Variants | Amino Acid Sequence | NO |
| E350W | MHHHHHHVNSNTINIAKNDFSDIELAAIPFNTLADHYGERLAREQLALEHESY | 10 |
| D351V | EMGEARFRKMFERQLKAGEVADNAAAKPLITTLLPKMIARINDWFEEVKAKRG | |
| K387S | KRPTAFQFLQEIKPEAVAYITIKTTLACLTSADNTTVQAVASAIGRAIEDEAR | |
| N437Y | FGRIRDLEAKHFKKNVEEQLNKRVGHVYKKAFMQVVEADMLSKGLLGGEAWSS | |
| | WHKEDSIHVGVRCIEMLIESTGMVSLHRQNAGVVGQDSETIELAPEYAEAIAT | |
| | RAGALAGISPMFQPCVVPPKPWTGITGGGYWANGRRPLALVRTHSKKALMRYE | |
| | DVYMPEVYKAINIAQNTAWKINKKVLAVANVITKWKHCPVWVIPAIEREELPM | |
| | KPEDIDMNPEALTAWKRAAAAVYRSDKARKSRRISLEFMLEQANKFANHKAIW | |
| | FPYNMDWRGRVYAVSMFNPQGYDMTKGLLTLAKGKPIGKEGYYWLKIHGANCA | |
| | GVDKVPFPERIKFIEENHENIMACAKSPLENTWWAEQDSPFCFLAFCFEYAGV | |
| | QHHGLSYNCSLPLAFDGSCSGIQHFSAMLRDEVGGRAVNLLPSETVQDIYGIV | |
| | AKKVNEILQADAINGTDNEVVTVTDENTGEISEKVKLGTKALAGQWLAYGVTR | |
| | SVTKRSVMTLAYGSKEFGFRQQVLEDTIQPAIDSGKGLMFTQPNQAAGYMAKL | |
| | IWESVSVTVVAAVEAMNWLKSAAKLLAAEVKDKKTGEILRKRCAVHWVTPDGF | |
| | PVWQEYKKPIQTRLNLMFLGQFRLQPTINTNKDSEIDAHKQESGIAPNFVHSQ | |
| | DGSHLRKTVVWAHEKYGIESFALIHDSFGTIPADAANLFKAVRETMVDTYESC | |
| | DVLADFYDQFADQLHESQLDKMPALPAKGNLNLRDILESDFAFA | |
| N437Y | MHHHHHHVNSNTINIAKNDFSDIELAAIPFNTLADHYGERLAREQLALEHESY | 11 |
| E350W | EMGEARFRKMFERQLKAGEVADNAAAKPLITTLLPKMIARINDWFEEVKAKRG | |
| D351V | KRPTAFQFLQEIKPEAVAYITIKTTLACLTSADNTTVQAVASAIGRAIEDEAR | |
| | FGRIRDLEAKHFKKNVEEQLNKRVGHVYKKAFMQVVEADMLSKGLLGGEAWSS | |
| | WHKEDSIHVGVRCIEMLIESTGMVSLHRQNAGVVGQDSETIELAPEYAEAIAT | |
| | RAGALAGISPMFQPCVVPPKPWTGITGGGYWANGRRPLALVRTHSKKALMRYE | |
| | DVYMPEVYKAINIAQNTAWKINKKVLAVANVITKWKHCPVWVIPAIEREELPM | |
| | KPEDIDMNPEALTAWKRAAAAVYRKDKARKSRRISLEFMLEQANKFANHKAIW | |
| | FPYNMDWRGRVYAVSMFNPQGYDMTKGLLTLAKGKPIGKEGYYWLKIHGANCA | |
| | GVDKVPFPERIKFIEENHENIMACAKSPLENTWWAEQDSPFCFLAFCFEYAGV | |
| | QHHGLSYNCSLPLAFDGSCSGIQHFSAMLRDEVGGRAVNLLPSETVQDIYGIV | |
| | AKKVNEILQADAINGTDNEVVTVTDENTGEISEKVKLGTKALAGQWLAYGVTR | |
| | SVTKRSVMTLAYGSKEFGFRQQVLEDTIQPAIDSGKGLMFTQPNQAAGYMAKL | |
| | IWESVSVTVVAAVEAMNWLKSAAKLLAAEVKDKKTGEILRKRCAVHWVTPDGF | |
| | PVWQEYKKPIQTRLNLMFLGQFRLQPTINTNKDSEIDAHKQESGIAPNFVHSQ | |
| | DGSHLRKTVVWAHEKYGIESFALIHDSFGTIPADAANLFKAVRETMVDTYESC | |
| | DVLADFYDQFADQLHESQLDKMPALPAKGNLNLRDILESDFAFA | |
| K387S | MHHHHHHVNSNTINIAKNDESDIELAAIPFNTLADHYGERLAREQLALEHESY | 12 |
| E350W | EMGEARFRKMFERQLKAGEVADNAAAKPLITTLLPKMIARINDWFEEVKAKRG | |
| D351V | KRPTAFQFLQEIKPEAVAYITIKTTLACLTSADNTTVQAVASAIGRAIEDEAR | |
| | FGRIRDLEAKHFKKNVEEQLNKRVGHVYKKAFMQVVEADMLSKGLLGGEAWSS | |
| | WHKEDSIHVGVRCIEMLIESTGMVSLHRQNAGVVGQDSETIELAPEYAEAIAT | |
| | RAGALAGISPMFQPCVVPPKPWTGITGGGYWANGRRPLALVRTHSKKALMRYE | |
| | DVYMPEVYKAINIAQNTAWKINKKVLAVANVITKWKHCPVWVIPAIEREELPM | |
| | KPEDIDMNPEALTAWKRAAAAVYRSDKARKSRRISLEFMLEQANKFANHKAIW | |
| | FPYNMDWRGRVYAVSMFNPQGNDMTKGLLTLAKGKPIGKEGYYWLKIHGANCA | |
| | GVDKVPFPERIKFIEENHENIMACAKSPLENTWWAEQDSPFCFLAFCFEYAGV | |
| | QHHGLSYNCSLPLAFDGSCSGIQHFSAMLRDEVGGRAVNLLPSETVQDIYGIV | |
| | AKKVNEILQADAINGTDNEVVTVTDENTGEISEKVKLGTKALAGQWLAYGVTR | |
| | SVTKRSVMTLAYGSKEFGFRQQVLEDTIQPAIDSGKGLMFTQPNQAAGYMAKL | |
| | IWESVSVTVVAAVEAMNWLKSAAKLLAAEVKDKKTGEILRKRCAVHWVTPDGF | |
| | PVWQEYKKPIQTRLNLMFLGQFRLQPTINTNKDSEIDAHKQESGIAPNFVHSQ | |
| | DGSHLRKTVVWAHEKYGIESFALIHDSFGTIPADAANLFKAVRETMVDTYESC | |
| | DVLADFYDQFADQLHESQLDKMPALPAKGNLNLRDILESDFAFA | |
| D653W | MHHHHHHVNSNTINIAKNDESDIELAAIPFNTLADHYGERLAREQLALEHESY | 13 |
| E350W | EMGEARFRKMFERQLKAGEVADNAAAKPLITTLLPKMIARINDWFEEVKAKRG | |
| D351V | KRPTAFQFLQEIKPEAVAYITIKTTLACLTSADNTTVQAVASAIGRAIEDEAR | |
| | FGRIRDLEAKHFKKNVEEQLNKRVGHVYKKAFMQVVEADMLSKGLLGGEAWSS | |
| | WHKEDSIHVGVRCIEMLIESTGMVSLHRQNAGVVGQDSETIELAPEYAEAIAT | |
| | RAGALAGISPMFQPCVVPPKPWTGITGGGYWANGRRPLALVRTHSKKALMRYE | |
| | DVYMPEVYKAINIAQNTAWKINKKVLAVANVITKWKHCPVWVIPAIEREELPM | |
| | KPEDIDMNPEALTAWKRAAAAVYRKDKARKSRRISLEFMLEQANKFANHKAIW | |
| | FPYNMDWRGRVYAVSMFNPQGNDMTKGLLTLAKGKPIGKEGYYWLKIHGANCA | |
| | GVDKVPFPERIKFIEENHENIMACAKSPLENTWWAEQDSPFCFLAFCFEYAGV | |
| | QHHGLSYNCSLPLAFDGSCSGIQHFSAMLRDEVGGRAVNLLPSETVQDIYGIV | |
| | AKKVNEILQADAINGTDNEVVTVTDENTGEISEKVKLGTKALAGQWLAYGVTR | |
| | SVTKRSVMTLAYGSKEFGFRQQVLEWTIQPAIDSGKGLMFTQPNQAAGYMAKL | |
| | IWESVSVTVVAAVEAMNWLKSAAKLLAAEVKDKKTGEILRKRCAVHWVTPDGF | |
| | PVWQEYKKPIQTRLNLMFLGQFRLQPTININKDSEIDAHKQESGIAPNFVHSQ | |
| | DGSHLRKTVVWAHEKYGIESFALIHDSFGTIPADAANLFKAVRETMVDTYESC | |
| | DVLADFYDQFADQLHESQLDKMPALPAKGNLNLRDILESDFAFA | |

In some embodiments, RNA polymerase variants have at least 90%, at least 91%, at least 92%, at least 93%, at least 94%, at least 95%, at least 96%, at least 97%, at least 98%, or at least 99% identity with an RNA polymerase comprising the amino acid sequence of any one of SEQ ID NOs: 2-13. In some embodiments, RNA polymerase variants may share at least 90%, at least 91%, at least 92%, at least 93%, at least 94%, at least 95% identity with an RNA polymerase comprising the amino acid sequence of SEQ ID NO: 1.

The term “identity” refers to a relationship between the sequences of two or more polypeptides (e.g. enzymes) or polynucleotides (nucleic acids), as determined by comparing the sequences. Identity also refers to the degree of sequence relatedness between or among sequences as determined by the number of matches between strings of two or more amino acid residues or nucleic acid residues. Identity measures the percent of identical matches between the smaller of two or more sequences with gap alignments (if any) addressed by a particular mathematical model or computer program (e.g., “algorithms”). Identity of related proteins or nucleic acids can be readily calculated by known methods. “Percent (%) identity” as it applies to polypeptide or polynucleotide sequences is defined as the percentage of residues (amino acid residues or nucleic acid residues) in the candidate amino acid or nucleic acid sequence that are identical with the residues in the amino acid sequence or nucleic acid sequence of a second sequence after aligning the sequences and introducing gaps, if necessary, to achieve the maximum percent identity. Methods and computer programs for the alignment are well known in the art. It is understood that identity depends on a calculation of percent identity but may differ in value due to gaps and penalties introduced in the calculation. Generally, variants of a particular polynucleotide or polypeptide (e.g., antigen) have at least 40%, 45%, 50%, 55%, 60%, 65%, 70%, 75%, 80%, 85%, 90%, 91%, 92%, 93%, 94%, 95%, 96%, 97%, 98%, 99% but less than 100% sequence identity to that particular reference polynucleotide or polypeptide as determined by sequence alignment programs and parameters described herein and known to those skilled in the art. Such tools for alignment include those of the BLAST suite (Stephen F. Altschul, et al (1997), “Gapped BLAST and PSI-BLAST: a new generation of protein database search programs”, Nucleic Acids Res. 25:3389-3402). Another popular local alignment technique is based on the Smith-Waterman algorithm (Smith, T.F. & Waterman, M.S. (1981) “Identification of common molecular subsequences.” J. Mol. Biol. 147:195-197). A general global alignment technique based on dynamic programming is the Needleman-Wunsch algorithm (Needleman, S.B. & Wunsch, C.D. (1970) “A general method applicable to the search for similarities in the amino acid sequences of two proteins.” J. Mol. Biol. 48:443-453). More recently a Fast Optimal Global Sequence Alignment Algorithm (FOGSAA) has been developed that purportedly produces global alignment of nucleotide and protein sequences faster than other optimal global alignment methods, including the Needleman-Wunsch algorithm.

### RNA Capping

Multivalent RNA compositions may comprise one or more mRNAs having open reading frames that encode proteins or peptides. Each of these mRNAs may have a 5′ Cap. The 5′ Cap may be added during the co-IVT reaction (e.g., transcriptional co-capping) or after the IVT reaction.

Some aspects also include a polynucleotide that comprises both a 5′ Cap and a polynucleotide (e.g., a polynucleotide comprising a nucleotide sequence encoding a polypeptide to be expressed).

The 5′ cap structure of a natural mRNA is involved in nuclear export, increasing mRNA stability and binds the mRNA Cap Binding Protein (CBP), for example eIF4E, which is responsible for mRNA stability in the cell and translation competency through the association of CBP with poly(A) binding protein to form the mature cyclic mRNA species. The cap further assists the removal of 5′ proximal introns during mRNA splicing.

Endogenous mRNA molecules can be 5′-end capped generating a 5′-ppp-5′-triphosphate linkage between a terminal guanosine cap residue and the 5′-terminal transcribed sense nucleotide of the mRNA molecule. This 5′-guanylate cap can then be methylated to generate an N7-methyl-guanylate residue. The ribose sugars of the terminal and/or anteterminal transcribed nucleotides of the 5′ end of the mRNA can optionally also be 2′-O-methylated. 5′-decapping through hydrolysis and cleavage of the guanylate cap structure can target a nucleic acid molecule, such as an mRNA molecule, for degradation.

In some embodiments, the polynucleotides (e.g., a polynucleotide comprising a nucleotide sequence encoding a polypeptide) incorporate a cap moiety.

In some embodiments, polynucleotides comprise a non-hydrolyzable cap structure preventing decapping and thus increasing mRNA half-life. Because cap structure hydrolysis requires cleavage of 5′-ppp-5′ phosphodiester linkages, modified nucleotides can be used during the capping reaction. For example, a Vaccinia Capping Enzyme from New England Biolabs (Ipswich, MA) can be used with α-thio-guanosine nucleotides according to the manufacturer's instructions to create a phosphothioate linkage in the 5′-ppp-5′ cap. Additional modified guanosine nucleotides can be used such as α-methyl-phosphonate and seleno-phosphate nucleotides.

Additional modifications include, but are not limited to, 2′-O-methylation of the ribose sugars of 5′-terminal and/or 5′-anteterminal nucleotides of the polynucleotide (as mentioned above) on the 2′-hydroxyl group of the sugar ring. Multiple distinct 5′-cap structures can be used to generate the 5′-cap of a nucleic acid molecule, such as a polynucleotide that functions as an mRNA molecule. Cap analogs, which herein are also referred to as synthetic cap analogs, chemical caps, chemical cap analogs, or structural or functional cap analogs, differ from natural (i.e., endogenous, wild-type or physiological) 5′-caps in their chemical structure, while retaining cap function. Cap analogs can be chemically (i.e., non-enzymatically) or enzymatically synthesized and/or linked to the polynucleotides.

For example, the Anti-Reverse Cap Analog (ARCA) cap contains two guanines linked by a 5′-5′-triphosphate group, wherein one guanine contains an N7 methyl group as well as a 3′-O-methyl group (i.e., N7,3′-O-dimethyl-guanosine-5′-triphosphate-5′-guanosine (m⁷G-3′mppp-G; which can equivalently be designated 3′ O-Me-m⁷G(5′)ppp(5′)G). The 3′-O atom of the other, unmodified, guanine becomes linked to the 5′-terminal nucleotide of the capped polynucleotide. The N7- and 3′-O-methlyated guanine provides the terminal moiety of the capped polynucleotide.

Another exemplary cap is mCAP, which is similar to ARCA but has a 2′-O-methyl group on guanosine (i.e., N7,2′-O-dimethyl-guanosine-5′-triphosphate-5′-guanosine, m⁷Gm-ppp-G).

Another exemplary cap is m⁷G-ppp-Gm-AG (i.e., N7,guanosine-5′-triphosphate-2′-O-dimethyl-guanosine-adenosine-guanosine).

In some embodiments, the cap is a dinucleotide cap analog. As a non-limiting example, the dinucleotide cap analog can be modified at different phosphate positions with a boranophosphate group or a phosphoroselenoate group such as the dinucleotide cap analogs described in U.S. Pat. No. 8,519,110, the contents of which are herein incorporated by reference in its entirety.

In another embodiment, the cap is a cap analog is a N7-(4-chlorophenoxyethyl) substituted dinucleotide form of a cap analog known in the art and/or described herein. Non-limiting examples of a N7-(4-chlorophenoxyethyl) substituted dinucleotide form of a cap analog include a N7-(4-chlorophenoxyethyl)-G(5′)ppp(5′)G and a N7-(4-chlorophenoxyethyl)-m^{Y}-^{O}G(5′)ppp(5′)G cap analog (See, e.g., the various cap analogs and the methods of synthesizing cap analogs described in Kore et al. Bioorganic & Medicinal Chemistry 2013 21:4570-4574; the contents of which are herein incorporated by reference in its entirety). In another embodiment, a cap analog is a 4-chloro/bromophenoxyethyl analog.

Polynucleotides can also be capped post-manufacture (whether IVT or chemical synthesis), using enzymes, in order to generate more authentic 5′-cap structures. As used herein, the phrase “more authentic” refers to a feature that closely mirrors or mimics, either structurally or functionally, an endogenous or wild type feature. That is, a “more authentic” feature is better representative of an endogenous, wild-type, natural or physiological cellular function and/or structure as compared to synthetic features or analogs, etc., of the prior art, or which outperforms the corresponding endogenous, wild-type, natural or physiological feature in one or more respects. Non-limiting examples of more authentic 5′cap structures are those that, among other things, have enhanced binding of cap binding proteins, increased half-life, reduced susceptibility to 5′ endonucleases and/or reduced 5′decapping, as compared to synthetic 5′cap structures known in the art (or to a wild-type, natural or physiological 5′cap structure). For example, recombinant Vaccinia Virus Capping Enzyme and recombinant 2′-O-methyltransferase enzyme can create a canonical 5′-5′-triphosphate linkage between the 5′-terminal nucleotide of a polynucleotide and a guanine cap nucleotide wherein the cap guanine contains an N7 methylation and the 5′-terminal nucleotide of the mRNA contains a 2′-O-methyl. Such a structure is termed the CapI structure. This cap results in a higher translational-competency and cellular stability and a reduced activation of cellular pro-inflammatory cytokines, as compared, e.g., to other 5′cap analog structures known in the art. Cap structures include, but are not limited to, 7mG(5′)ppp(5′)N,pN2p (cap 0), 7mG(5′)ppp(5′)NlmpNp (cap 1), and 7mG(5′)-ppp(5′)NlmpN2mp (cap 2).

As a non-limiting example, capping chimeric polynucleotides post-manufacture can be more efficient as nearly 100% of the chimeric polynucleotides can be capped. This is in contrast to ˜80% when a cap analog is linked to a chimeric polynucleotide in the course of an in vitro transcription reaction.

In some embodiments, 5′ terminal caps can include endogenous caps or cap analogs. In some embodiments, a 5′ terminal cap can comprise a guanine analog. Useful guanine analogs include, but are not limited to, inosine, N1-methyl-guanosine, 2′fluoro-guanosine, 7-deaza-guanosine, 8-oxo-guanosine, 2-amino-guanosine, LNA-guanosine, and 2-azido-guanosine.

Also described are exemplary caps including those that can be used in co-transcriptional capping methods for ribonucleic acid (RNA) synthesis, using RNA polymerase, e.g., wild type RNA polymerase or variants thereof, e.g., such as those variants described. In one embodiment, caps can be added when RNA is produced in a “one-pot” reaction, without the need for a separate capping reaction. Thus, the methods, in some embodiments, comprise reacting a polynucleotide template with a RNA polymerase variant, nucleoside triphosphates, and a cap analog under in vitro transcription reaction conditions to produce RNA transcript.

In some embodiments, the cap analog binds to a polynucleotide template that comprises a promoter region comprising a transcriptional start site having a first nucleotide at nucleotide position +1, a second nucleotide at nucleotide position +2, and a third nucleotide at nucleotide position +3. In some embodiments, the cap analog hybridizes to the polynucleotide template at least at nucleotide position +1, such as at the +1 and +2 positions, or at the +1, +2, and +3 positions.

A cap analog may be, for example, a dinucleotide cap, a trinucleotide cap, or a tetranucleotide cap. In some embodiments, a cap analog is a dinucleotide cap. In some embodiments, a cap analog is a trinucleotide cap. In some embodiments, a cap analog is a tetranucleotide cap. As used here the term “cap” includes the inverted G nucleotide and can comprise additional nucleotides 3′ of the inverted G, .e.g., 1, 2, or more nucleotides 3′ of the inverted G and 5′ to the 5′ UTR.

Exemplary caps comprise a sequence GG, GA, or GGA wherein the underlined, italicized G is an inverted G.

A nucleotide cap (e.g., a trinucleotide cap or tetranucleotide cap), in some embodiments, comprises a compound of formula (I)

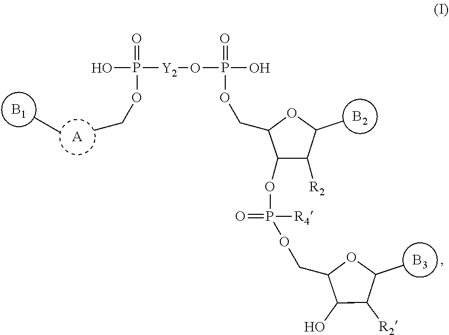

(or a stereoisomer, tautomer or salt thereof, wherein

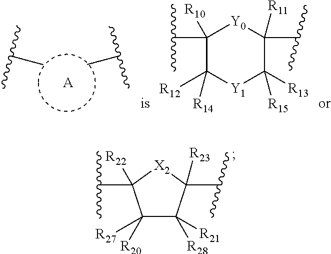

ring B₁ is a modified or unmodified Guanine;
ring B₂ and ring B₃ each independently is a nucleobase or a modified nucleobase;
X₂ is O, S(O)ₚ, NR₂₄ or CR₂₅R₂₆ in which p is 0, 1, or 2;
Y₀ is O or CR₆R₇;
Y1 is O, S(O)n, CR₆R7, or NR₈, in which n is 0, 1, or 2;
each [symbol] is a single bond or absent, wherein when each [symbol] is a single bond, Yi is O, S(O) , CR₆R₇, or NR₈; and when each [symbol] is absent, Y₁ is void;
Y₂ is (OP(O)R₄)ₘ in which m is 0, 1, or 2, or —O—(CR₄₀R₄₁)u-Q₀-(CR₄₂R₄₃)ᵥ—, in which Q₀ is a bond, O, S(O)ᵣ, NR₄₄, or CR₄₅R₄₆, r is 0, 1, or 2, and each of u and v independently is 1, 2, 3 or 4;
each R₂ and R₂′ independently is halo, LNA, or OR₃;
each R₃ independently is H, C₁-C₆ alkyl, C₂-C₆ alkenyl, or C₂-C₆ alkynyl and R₃, when being C₁-C₆ alkyl, C₂-C₆ alkenyl, or C₂-C₆ alkynyl, is optionally substituted with one or more of halo, OH and C₁-C₆ alkoxyl that is optionally substituted with one or more OH or OC(O)—C₁-C₆ alkyl;
each R₄ and R₄′ independently is H, halo, C₁-C₆ alkyl, OH, SH, SeH, or BH₃⁻;
each of R₆, R₇, and R₈, independently, is -Q₁-T₁, in which Q₁ is a bond or C₁-C₃ alkyl linker optionally substituted with one or more of halo, cyano, OH and C₁-C₆ alkoxy, and Ti is H, halo, OH, COOH, cyano, or Rₛ₁, in which Rₛ₁ is C₁-C₃ alkyl, C₂-C₆ alkenyl, C₂-C₆ alkynyl, C₁-C₆ alkoxyl, C(O)O—C₁-C₆ alkyl, C₃-C₈ cycloalkyl, C₆-C₁₀ aryl, NR₃₁R₃₂, (NR₃₁R₃₂R₃₃)⁺, 4 to 12-membered heterocycloalkyl, or 5- or 6-membered heteroaryl, and Rₛ₁ is optionally substituted with one or more substituents selected from the group consisting of halo, OH, oxo, C₁-C₆ alkyl, COOH, C(O)O—C₁-C₆ alkyl, cyano, C₁-C₆ alkoxyl, NR₃₁R₃₂, (NR₃₁R₃₂R₃₃)⁺, C₃-C₈ cycloalkyl, C₆-C₁₀ aryl, 4 to 12-membered heterocycloalkyl, and 5- or 6-membered heteroaryl;
each of R₁₀, R¹¹, R₁₂, R₁₃ R₁₄, and R₁₅, independently, is -Q₂-T₂, in which Q₂ is a bond or C₁-C₃ alkyl linker optionally substituted with one or more of halo, cyano, OH and C₁-C₆ alkoxy, and T₂ is H, halo, OH, NH₂, cyano, NO₂, N₃, Rₛ₂, or ORₛ₂, in which Rₛ₂ is C₁-C₆ alkyl, C₂-C₆ alkenyl, C₂-C₆ alkynyl, C₃-C₈ cycloalkyl, C₆-C₁₀ aryl, NHC(O)—C₁-C₆ alkyl, NR₃₁R₃₂, (NR₃₁R32R₃₃)+, 4 to 12-membered heterocycloalkyl, or 5- or 6-membered heteroaryl, and Rₛ₂ is optionally substituted with one or more substituents selected from the group consisting of halo, OH, oxo, C₁-C₆ alkyl, COOH, C(O)O—C₁-C₆ alkyl, cyano, C₁-C₆ alkoxyl, NR₃₁R₃₂, (NR₃₁R₃₂R₃₃)⁺, C₃-C₈ cycloalkyl, C₆-C₁₀ aryl, 4 to 12-membered heterocycloalkyl, and 5- or 6-membered heteroaryl; or alternatively R₁₂ together with R₁₄ is oxo, or R₁₃ together with R₁₅ is oxo,
each of R₂₀, R₂₁, R₂₂, and R₂₃ independently is -Q₃-T₃, in which Q₃ is a bond or C₁-C₃ alkyl linker optionally substituted with one or more of halo, cyano, OH and C₁-C₆ alkoxy, and T₃ is H, halo, OH, NH₂, cyano, NO₂, N₃, Rₛ₃, or ORₛ₃, in which Rₛ₃ is C₁-C₆ alkyl, C₂-C₆ alkenyl, C₂-C₆ alkynyl, C₃-C₈ cycloalkyl, C₆-C₁₀ aryl, NHC(O)—C₁-C₆ alkyl, mono-C₁-C₆ alkylamino, di-C₁-C₆ alkylamino, 4 to 12-membered heterocycloalkyl, or 5- or 6-membered heteroaryl, and Rₛ₃ is optionally substituted with one or more substituents selected from the group consisting of halo, OH, oxo, C₁-C₆ alkyl, COOH, C(O)O—C₁-C₆ alkyl, cyano, C₁-C₆ alkoxyl, amino, mono-C₁-C₆ alkylamino, di-C₁-C₆ alkylamino, C₃-C₈ cycloalkyl, C₆-C₁₀ aryl, 4 to 12-membered heterocycloalkyl, and 5- or 6-membered heteroaryl;
   each of R₂₄, R₂₅, and R₂₆ independently is H or C₁-C₆ alkyl;
each of R₂₇ and R₂₈ independently is H or OR₂₉; or R₂₇ and R₂₈ together form O—R₃₀—O; each R₂₉ independently is H, C₁-C₆ alkyl, C₂-C₆ alkenyl, or C₂-C₆ alkynyl and R₂₉, when being C₁-C₆ alkyl, C₂-C₆ alkenyl, or C₂-C₆ alkynyl, is optionally substituted with one or more of halo, OH and C₁-C₆ alkoxyl that is optionally substituted with one or more OH or OC(O)—C₁-C₆ alkyl;
R₃₀ is C₁-C₆ alkylene optionally substituted with one or more of halo, OH and C₁-C₆ alkoxyl;
each of R₃₁, R₃₂, and R₃₃, independently is H, C₁-C₆ alkyl, C₃-C₈ cycloalkyl, C₆-C₁₀ aryl, 4 to 12-membered heterocycloalkyl, or 5- or 6-membered heteroaryl;
each of R₄₀, R₄₁, R₄₂, and R₄₃ independently is H, halo, OH, cyano, N₃, OP(O)R₄₇R₄₈, or C₁-C₆ alkyl optionally substituted with one or more OP(O)R₄₇R₄₈, or one R₄₁ and one R₄₃, together with the carbon atoms to which they are attached and Q₀, form C₄-C₁₀ cycloalkyl, 4- to 14-membered heterocycloalkyl, C₆-C₁₀ aryl, or 5- to 14-membered heteroaryl, and each of the cycloalkyl, heterocycloalkyl, phenyl, or 5- to 6-membered heteroaryl is optionally substituted with one or more of OH, halo, cyano, N₃, oxo, OP(O)R₄₇R₄₈, C₁-C₆ alkyl, C₁-C₆ haloalkyl, COOH, C(O)O—C₁-C₆ alkyl, C₁-C₆ alkoxyl, C₁-C₆ haloalkoxyl, amino, mono-C₁-C₆ alkylamino, and di-C₁-C₆ alkylamino;
R₄₄ is H, C₁-C₆ alkyl, or an amine protecting group;
each of R₄₅ and R₄₆ independently is H, OP(O)R₄₇R₄₈, or C₁-C₆ alkyl optionally substituted with one or more OP(O)R₄₇R₄₈, and
each of R₄₇ and R₄₈, independently is H, halo, C₁-C₆ alkyl, OH, SH, SeH, or BH₃⁻.

It should be understood that a cap analog may include any of the cap analogs described in international publication WO 2017/066797, published on 20 Apr. 2017, incorporated by reference herein in its entirety.

In some embodiments, the B₂ middle position can be a non-ribose molecule, such as arabinose.

In some embodiments R₂ is ethyl-based.

Thus, in some embodiments, a trinucleotide cap comprises the following structure:

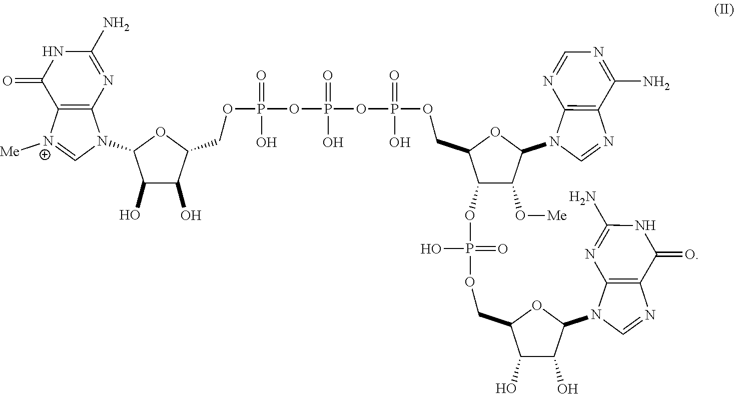 [SMILES: CO[C@@H]1[C@H](OP(=O)(O)OC[C@H]2O[C@@H](n3cnc4c(=O)[nH]c(N)nc43)[C@H](O)[C@@H]2O)[C@@H](COP(=O)(O)OP(=O)(O)OP(=O)(O)OC[C@H]2O[C@@H](n3c[n+](C)c4c(=O)[nH]c(N)nc43)[C@H](O)[C@@H]2O)O[C@H]1n1cnc2c(N)ncnc21]

In other embodiments, a trinucleotide cap comprises the following structure:

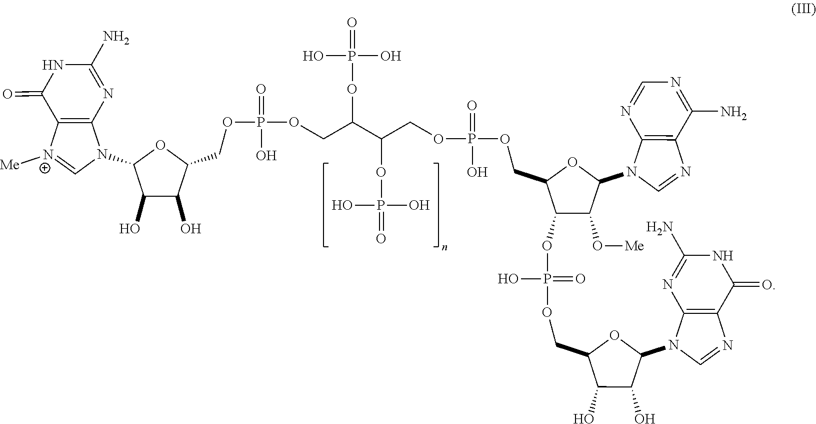

In yet other embodiments, a trinucleotide cap comprises the following structure:

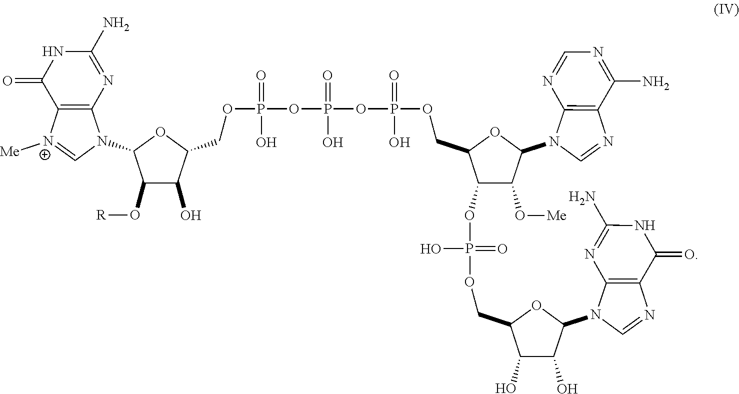

In still other embodiments, a trinucleotide cap comprises the following structure:

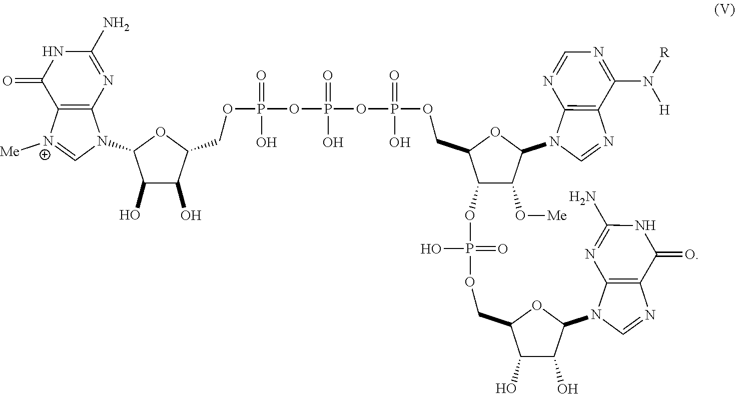

Thus, in some embodiments, a tetranucleotide cap comprises the following structure:

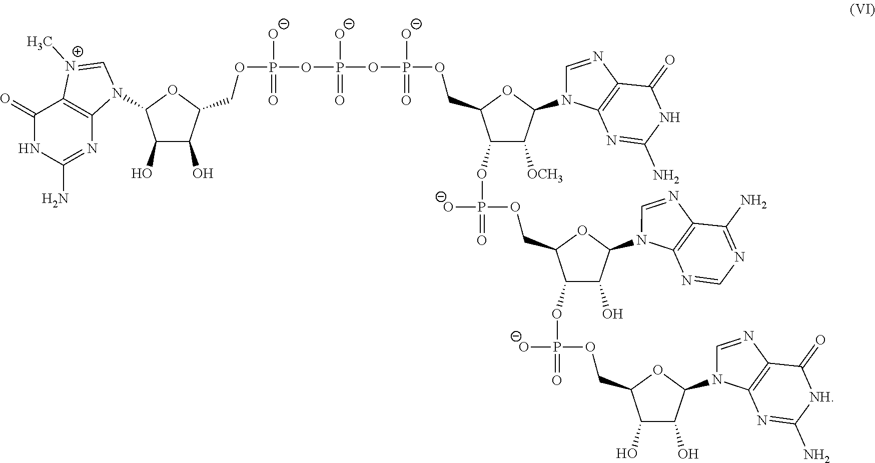

In other embodiments, a tetranucleotide cap comprises the following structure:

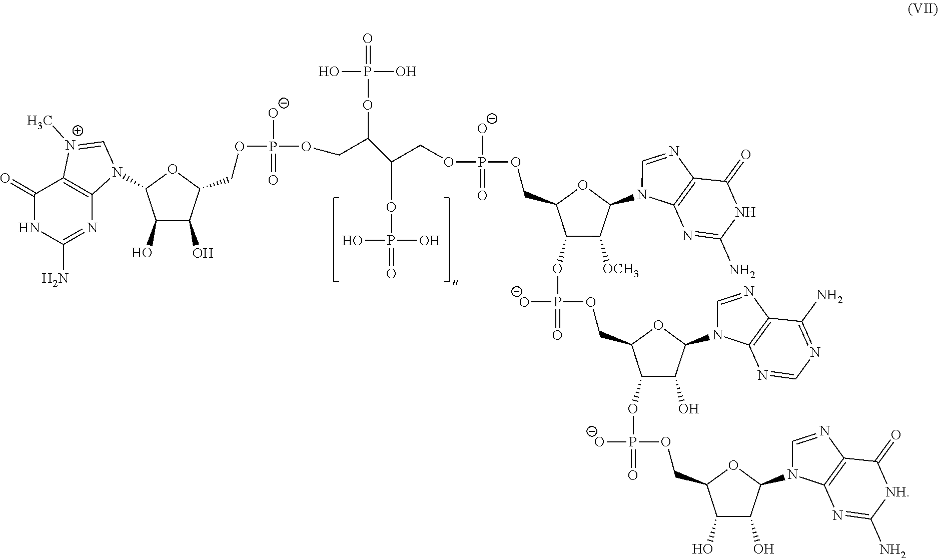

In yet other embodiments, a tetranucleotide cap comprises the following structure:

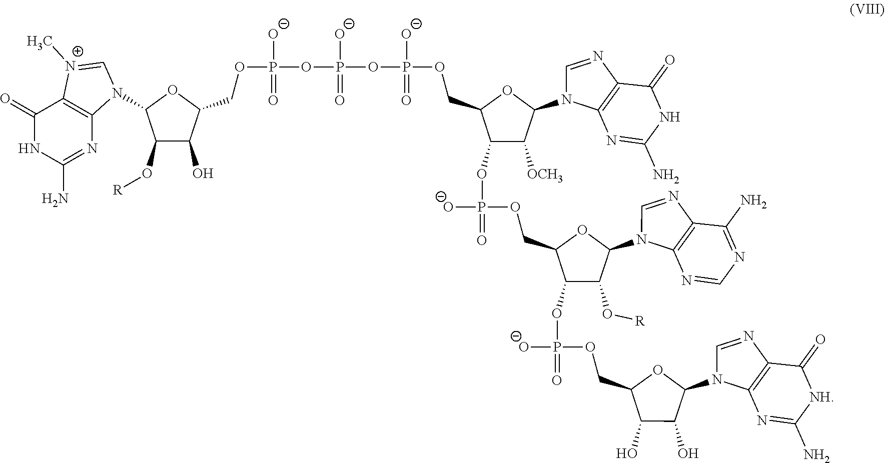

In yet other embodiments, a tetranucleotide cap comprises the following structure:

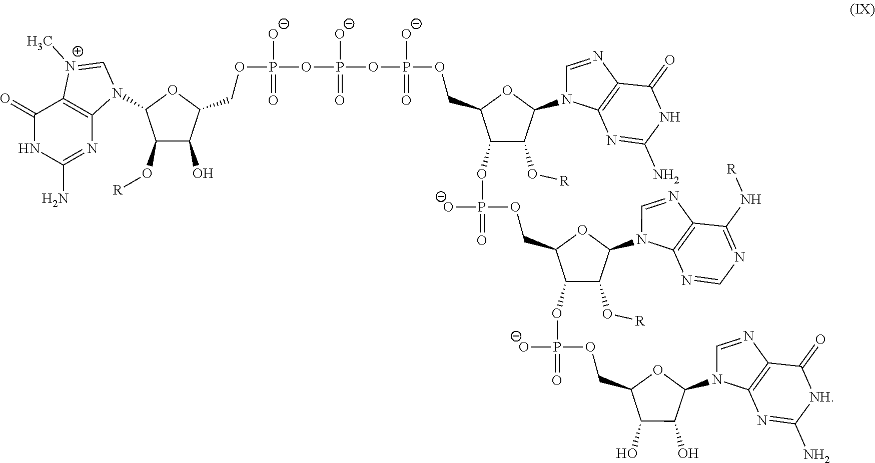

In some embodiments, R is an alkyl (e.g., C₁-C₆ alkyl). In some embodiments, R is a methyl group (e.g., C₁ alkyl). In some embodiments, R is an ethyl group (e.g., C₂ alkyl). In some embodiments, R is a hydrogen.

A trinucleotide cap, in some embodiments, comprises a sequence selected from the following sequences: GAA, GAC, GAG, GAU, GCA, GCC, GCG, GCU, GGA, GGC, GGG, GGU, GUA, GUC, GUG, and GUU. In some embodiments, a trinucleotide cap comprises GAA. In some embodiments, a trinucleotide cap comprises GAC. In some embodiments, a trinucleotide cap comprises GAG. In some embodiments, a trinucleotide cap comprises GAU. In some embodiments, a trinucleotide cap comprises GCA. In some embodiments, a trinucleotide cap comprises GCC. In some embodiments, a trinucleotide cap comprises GCG. In some embodiments, a trinucleotide cap comprises GCU. In some embodiments, a trinucleotide cap comprises GGA. In some embodiments, a trinucleotide cap comprises GGC. In some embodiments, a trinucleotide cap comprises GGG. In some embodiments, a trinucleotide cap comprises GGU. In some embodiments, a trinucleotide cap comprises GUA. In some embodiments, a trinucleotide cap comprises GUC. In some embodiments, a trinucleotide cap comprises GUG. In some embodiments, a trinucleotide cap comprises GUU.

In some embodiments, a trinucleotide cap comprises a sequence selected from the following sequences: m⁷GpppApA, m⁷GpppApC, m⁷GpppApG, m⁷GpppApU, m⁷GpppCpA, m⁷GpppCpC, m⁷GpppCpG, m⁷GpppCpU, m⁷GpppGpA, m⁷GpppGpC, m⁷GpppGpG, m⁷GpppGpU, m⁷GpppUpA, m⁷GpppUpC, m⁷GpppUpG, and m⁷GpppUpU.

In some embodiments, a trinucleotide cap comprises m⁷GpppApA. In some embodiments, a trinucleotide cap comprises m⁷GpppApC. In some embodiments, a trinucleotide cap comprises m⁷GpppApG. In some embodiments, a trinucleotide cap comprises m⁷GpppApU. In some embodiments, a trinucleotide cap comprises m⁷GpppCpA. In some embodiments, a trinucleotide cap comprises m⁷GpppCpC. In some embodiments, a trinucleotide cap comprises m⁷GpppCpG. In some embodiments, a trinucleotide cap comprises m⁷GpppCpU. In some embodiments, a trinucleotide cap comprises m⁷GpppGpA. In some embodiments, a trinucleotide cap comprises m⁷GpppGpC. In some embodiments, a trinucleotide cap comprises m⁷GpppGpG. In some embodiments, a trinucleotide cap comprises m⁷GpppGpU. In some embodiments, a trinucleotide cap comprises m⁷GpppUpA. In some embodiments, a trinucleotide cap comprises m⁷GpppUpC. In some embodiments, a trinucleotide cap comprises m⁷GpppUpG. In some embodiments, a trinucleotide cap comprises m⁷GpppUpU.

A trinucleotide cap, in some embodiments, comprises a sequence selected from the following sequences: m⁷G_{3′oMe}pppApA, m⁷G_{3′oMe}pppApC, m⁷G_{3′oMe}pppApG, m⁷G_{3′oMe}pppApU, m⁷G_{3′oMe}pppCpA, m⁷G_{3′oMe}pppCpC, m⁷G_{3′oMe}pppCpG, m⁷G_{3′oMe}pppCpU, m⁷G_{3′oMe}pppGpA, m⁷G_{3′oMe}pppGpC, m⁷G_{3′oMe}pppGpG, m⁷G_{3′oMe}pppGpU, m⁷G_{3′oMe}pppUpA, m⁷G_{3′oMe}pppUpC, m⁷G_{3′oMe}pppUpG, and m⁷G_{3′oMe}pppUpU.

In some embodiments, a trinucleotide cap comprises m⁷G_{3′oMe}pppApA. In some embodiments, a trinucleotide cap comprises m⁷G_{3′oMe}pppApC. In some embodiments, a trinucleotide cap comprises m⁷G_{3′oMe}pppApG. In some embodiments, a trinucleotide cap comprises m⁷G_{3′oMe}pppApU. In some embodiments, a trinucleotide cap comprises m⁷G_{3′oMe}pppCpA. In some embodiments, a trinucleotide cap comprises m⁷G3′O_{Me}pppCpC. In some embodiments, a trinucleotide cap comprises m⁷G_{3′oMe}pppCpG. In some embodiments, a trinucleotide cap comprises m⁷G_{3′oMe}pppCpU. In some embodiments, a trinucleotide cap comprises m⁷G_{3′oMe}pppGpA. In some embodiments, a trinucleotide cap comprises m⁷G_{3′oMe}pppGpC. In some embodiments, a trinucleotide cap comprises m⁷G_{3′OMe}pppGpG. In some embodiments, a trinucleotide cap comprises m⁷G_{3′oMe}pppGpU. In some embodiments, a trinucleotide cap comprises m⁷G_{3′oMe}pppUpA. In some embodiments, a trinucleotide cap comprises m⁷G_{3′oMe}pppUpC. In some embodiments, a trinucleotide cap comprises m⁷G_{3′oMe}pppUpG. In some embodiments, a trinucleotide cap comprises m⁷G3′O_{Me}pppUpU.

A trinucleotide cap, in other embodiments, comprises a sequence selected from the following sequences: m⁷G^{3′oMe}pppA_{2′OMe}pA, m⁷G_{3′oMe}pppA_{2′oMe}pC, m⁷G_{3′oMe}pppA_{2′oMe}pG, m⁷G_{3′oMe}pppA_{2′oMe}pU, m⁷G_{3′oMe}pppC_{2′oMe}pA, m⁷G_{3′oMe}pppC_{2′oMe}pC, m⁷G_{3′oMe}pppC_{2′oMe}pG, m⁷G_{3′oMe}pppC_{2′oMe}pU, m⁷G_{3′oMe}pppG_{2′oMep}A, m⁷G_{3′oMe}pppG_{2′oMe}pC, m⁷G_{3′oMe}pppG_{2′oMe}pG, m⁷G_{3′oMe}pppG_{2′oMe}pU, m⁷G_{3′oMe}pppU_{2′oMe}pA, m⁷G_{3′oMe}pppU_{2′oMe}pC, m⁷G_{3′oMe}pppU_{2′oMe}pG, and m⁷G_{3′oMe}pppU_{2′oMe}pU.

In some embodiments, a trinucleotide cap comprises m⁷G^{3′oMe}pppA_{2′oMe}pA. In some embodiments, a trinucleotide cap comprises m⁷G_{3′oMe}pppA_{2′oMe}pC. In some embodiments, a trinucleotide cap comprises m⁷G_{3′oMe}pppA_{2′oMe}pG. In some embodiments, a trinucleotide cap comprises m⁷G_{3′oMe}pppA_{2′oMe}pU. In some embodiments, a trinucleotide cap comprises m⁷G_{3′oMe}pppC_{2′oMe}pA. In some embodiments, a trinucleotide cap comprises m⁷G_{3′oMe}pppC_{2′oMe}pC. In some embodiments, a trinucleotide cap comprises m⁷G_{3′oMe}pppC_{2′oMe}pG. In some embodiments, a trinucleotide cap comprises m⁷G_{3′oMe}pppC_{2′oMe}pU. In some embodiments, a trinucleotide cap comprises m⁷G_{3′oMe}pppG_{2′oMe}pA. In some embodiments, a trinucleotide cap comprises m⁷G_{3′oMe}pppG_{2′oMe}pC. In some embodiments, a trinucleotide cap comprises m⁷G_{3′oMe}pppG_{2′oMe}pG. In some embodiments, a trinucleotide cap comprises m⁷G_{3′oMe}pppG_{2′oMe}pU. In some embodiments, a trinucleotide cap comprises m⁷G_{3′oMe}pppU_{2′oMe}pA. In some embodiments, a trinucleotide cap comprises m⁷G_{3′oMe}pppU_{2′oMe}pC. In some embodiments, a trinucleotide cap comprises m⁷G_{3′oMe}pppU_{2′oMe}pG. In some embodiments, a trinucleotide cap comprises m⁷G_{3′oMe}pppU_{2′oMe}pU.

A trinucleotide cap, in still other embodiments, comprises a sequence selected from the following sequences: m⁷GpppA_{2′OMe}pA, m⁷GpppA_{2′oMe}pC, m⁷GpppA_{2′oMe}pG, m⁷GpppA_{2′oMe}pU, m⁷GpppC_{2′oMe}pA, m⁷GpppC_{2′oMe}pC, m⁷GpppC_{2′oMe}pG, m⁷GpppC_{2′oMe}pU, m⁷GpppG_{2′oMe}pA, m⁷GpppG_{2′oMe}pC, m⁷GpppG_{2′oMe}pG, m⁷GpppG_{2′oMe}pU, m⁷GpppU_{2′oMe}pA, m⁷GpppU_{2′oMe}pC, m⁷GpppU_{2′oMe}pG, and m⁷GpppU_{2′oMe}pU.

In some embodiments, a trinucleotide cap comprises m⁷GpppA_{2′oMe}pA. In some embodiments, a trinucleotide cap comprises m⁷GpppA_{2′oMe}pC. In some embodiments, a trinucleotide cap comprises m⁷GpppA_{2′oMe}pG. In some embodiments, a trinucleotide cap comprises m⁷GpppA_{2′oMe}pU. In some embodiments, a trinucleotide cap comprises m⁷GpppC_{2′oMe}pA. In some embodiments, a trinucleotide cap comprises m⁷GpppC_{2′OMe}pC. In some embodiments, a trinucleotide cap comprises m⁷GpppC_{2′oMe}pG. In some embodiments, a trinucleotide cap comprises m⁷GpppC_{2′oMe}pU. In some embodiments, a trinucleotide cap comprises m⁷GpppG_{2′oMe}pA. In some embodiments, a trinucleotide cap comprises m⁷GpppG_{2′oMe}pC. In some embodiments, a trinucleotide cap comprises m⁷GpppG_{2′OMe}pG. In some embodiments, a trinucleotide cap comprises m⁷GpppG_{2′oMe}pU. In some embodiments, a trinucleotide cap comprises m⁷GpppU_{2′oMe}pA. In some embodiments, a trinucleotide cap comprises m⁷GpppU_{2′oMe}pC. In some embodiments, a trinucleotide cap comprises m⁷GpppU_{2′oMe}pG. In some embodiments, a trinucleotide cap comprises m⁷GpppU_{2′OMe}pU.

In some embodiments, a trinucleotide cap comprises m⁷Gpppm⁶A_{2′ome}pG. In some embodiments, a trinucleotide cap comprises m⁷Gpppe⁶A_{2′ome}pG.

In some embodiments, a trinucleotide cap comprises GAG. In some embodiments, a trinucleotide cap comprises GCG. In some embodiments, a trinucleotide cap comprises GUG. In some embodiments, a trinucleotide cap comprises GGG.

In some embodiments, a trinucleotide cap comprises any one of the following structures:

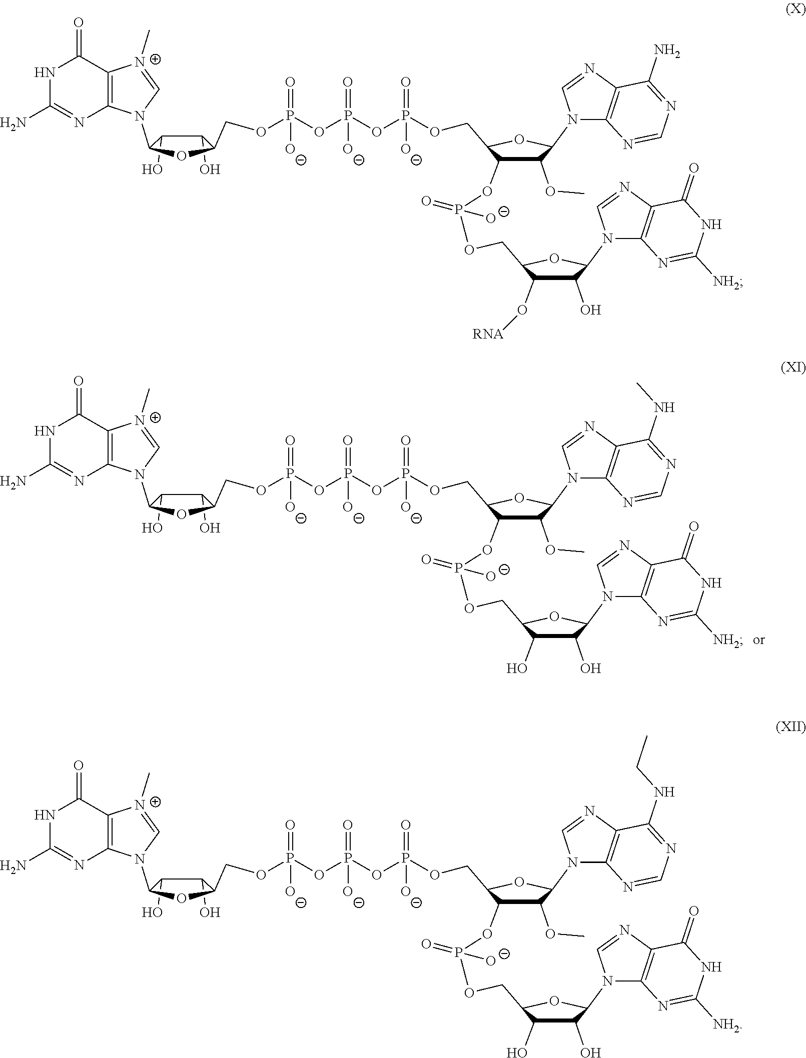

In some embodiments, the cap analog comprises a tetranucleotide cap. In some embodiments, the cap analog comprises GGAG.

In some embodiments, a tetranucleotide cap comprises any one of the following structures:

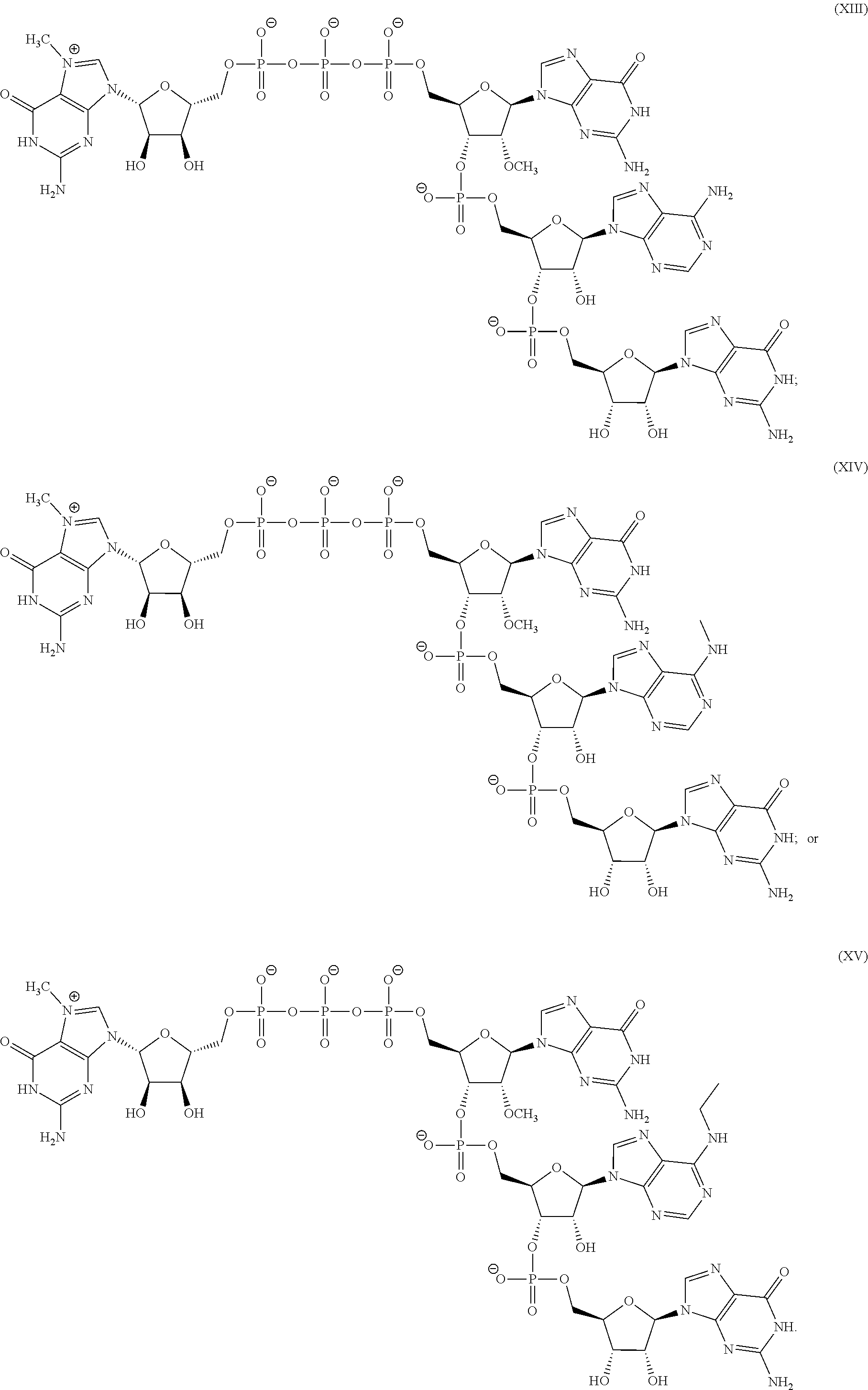

In some embodiments, the tetranucleotide cap comprises a trinucleotide as set forth above. In some embodiments, the tetranucleotide cap comprises ^{m7}GpppN₁N₂N₃, where N₁, N₂, and N₃ are optional (i.e., can be absent or one or more can be present) and are independently a natural, a modified, or an unnatural nucleoside base. In some embodiments, ^{m7}G is further methylated, e.g., at the 3′ position. In some embodiments, the ^{m7}G comprises an O-methyl at the 3′ position. In some embodiments N₁, N₂, and N₃ if present, optionally, are independently an adenine, a uracil, a guanidine, a thymine, or a cytosine. In some embodiments, one or more (or all) of N₁, N₂, and N₃, if present, are methylated, e.g., at the 2′ position. In some embodiments, one or more (or all) of N₁, N₂, and N₃, if present have an O-methyl at the 2′ position.

In some embodiments, the tetranucleotide cap comprises the following structure:

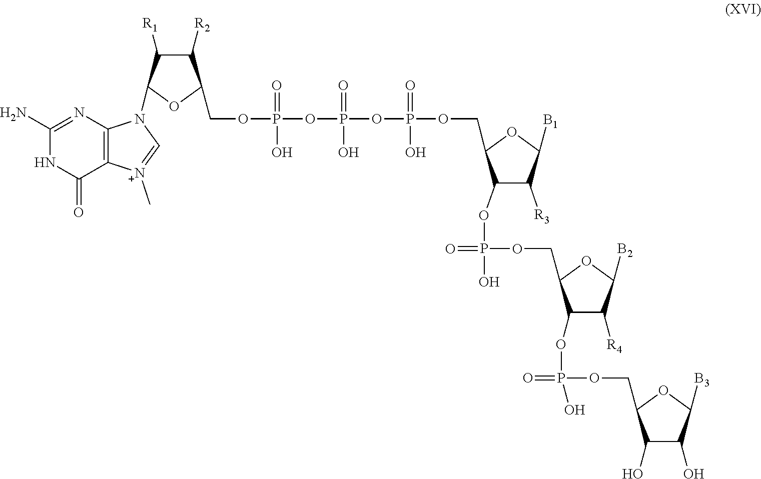

wherein B₁, B₂, and B₃ are independently a natural, a modified, or an unnatural nucleoside based; and R₁, R₂, R₃, and R₄ are independently OH or O-methyl. In some embodiments, R₃ is O-methyl and R₄ is OH. In some embodiments, R₃ and R₄ are O-methyl. In some embodiments, R₄ is O-methyl. In some embodiments, R₁ is OH, R₂ is OH, R₃ is O-methyl, and R₄ is OH. In some embodiments, R₁ is OH, R₂ is OH, R₃ is O-methyl, and R₄ is O-methyl. In some embodiments, at least one of R₁ and R₂ is O-methyl, R₃ is O-methyl, and R₄ is OH. In some embodiments, at least one of R₁ and R₂ is O-methyl, R₃ is O-methyl, and R₄ is O-methyl.

In some embodiments, B₁, B₃, and B₃ are natural nucleoside bases. In some embodiments, at least one of B₁, B₂, and B₃ is a modified or unnatural base. In some embodiments, at least one of B₁, B₂, and B₃ is N₆-methyladenine. In some embodiments, B₁ is adenine, cytosine, thymine, or uracil. In some embodiments, B₁ is adenine, B₂ is uracil, and B₃ is adenine. In some embodiments, R₁ and R₂ are OH, R₃ and R₄ are O-methyl, B₁ is adenine, B₂ is uracil, and B₃ is adenine.

In some embodiments the tetranucleotide cap comprises a sequence selected from the following sequences: GAAA, GACA, GAGA, GAUA, GCAA, GCCA, GCGA, GCUA, GGAA, GGCA, GGGA, GGUA, GUCA, and GUUA. In some embodiments the tetranucleotide cap comprises a sequence selected from the following sequences: GAAG, GACG, GAGG, GAUG, GCAG, GCCG, GCGG, GCUG, GGAG, GGCG, GGGG, GGUG, GUCG, GUGG, and GUUG. In some embodiments the tetranucleotide cap comprises a sequence selected from the following sequences: GAAU, GACU, GAGU, GAUU, GCAU, GCCU, GCGU, GCUU, GGAU, GGCU, GGGU, GGUU, GUAU, GUCU, GUGU, and GUUU. In some embodiments the tetranucleotide cap comprises a sequence selected from the following sequences: GAAC, GACC, GAGC, GAUC, GCAC, GCCC, GCGC, GCUC, GGAC, GGCC, GGGC, GGUC, GUAC, GUCC, GUGC, and GUUC.

A tetranucleotide cap, in some embodiments, comprises a sequence selected from the following sequences: m⁷G_{3′OMe}pppApApN, m⁷G_{3′oMe}pppApCpN, m⁷G_{3′oMe}pppApGpN, m⁷G_{3′oMe}pppApUpN, m⁷G_{3′oMe}pppCpApN, m⁷G_{3′oMe}pppCpCpN, m⁷G_{3′oMe}pppCpGpN, m⁷G_{3′oMe}pppCpUpN, m⁷G_{3′oMe}pppGpApN, m⁷G_{3′oMe}pppGpCpN, m⁷G_{3′oMe}pppGpGpN, m⁷G_{3′oMe}pppGpUpN, m⁷G_{3′oMe}pppUpApN, m⁷G_{3′oMe}pppUpCpN, m⁷G_{3′oMe}pppUpGpN, and m⁷G_{3′oMe}pppUpUpN, where N is a natural, a modified, or an unnatural nucleoside base.

A tetranucleotide cap, in other embodiments, comprises a sequence selected from the following sequences: m⁷G_{3′OMe}pppA_{2′OMe}pApN, m⁷G_{3′oMe}pppA_{2′oMe}pCpN, m⁷G_{3′oMe}pppA_{2′oMe}pGpN, m⁷G_{3′oMe}pppA_{2′oMe}pUpN, m⁷G_{3′oMe}pppC_{2′oMe}pApN, m⁷G_{3′oMe}pppC_{2′oMe}pCpN, m⁷G_{3′oMe}pppC_{2′oMe}pGpN, m⁷G_{3′oMe}pppC_{2′oMe}pUpN, m⁷G_{3′oMe}pppG_{2′oMep}ApN, m⁷G_{3′oMe}pppG_{2′oMe}pCpN, m⁷G_{3′oMe}pppG_{2′oMe}pGpN, m⁷G_{3′oMe}pppG_{2′oMe}pUpN, m⁷G_{3′oMe}pppU_{2′oMe}pApN, m⁷G_{3′oMe}pppU_{2′oMe}pCpN, m⁷G_{3′oMe}pppU_{2′oMe}pGpN, and m⁷G_{3′oMe}pppU_{2′oMe}pUpN, where N is a natural, a modified, or an unnatural nucleoside base.

A tetranucleotide cap, in still other embodiments, comprises a sequence selected from the following sequences: m⁷GpppA_{2′OMe}pApN, m⁷GpppA_{2′oMe}pCpN, m⁷GpppA_{2′oMe}pGpN, m⁷GpppA_{2′oMe}pUpN, m⁷GpppC_{2′oMe}pApN, m⁷GpppC_{2′oMe}pCpN, m⁷GpppC_{2′oMe}pGpN, m⁷GpppC_{2′oMe}pUpN, m⁷GpppG_{2′oMe}pApN, m⁷GpppG_{2′oMe}pCpN, m⁷GpppG_{2′oMe}pGpN, m⁷GpppG_{2′oMe}pUpN, m⁷GpppU_{2′oMe}pApN, m⁷GpppU_{2′oMe}pCpN, m⁷GpppU_{2′oMe}pGpN, and m⁷GpppU_{2′oMe}pUpN, where N is a natural, a modified, or an unnatural nucleoside base.

A tetranucleotide cap, in other embodiments, comprises a sequence selected from the following sequences: m⁷G_{3′OMe}pppA_{2′OMe}pA_{2′OMe}pN, m⁷G_{3′oMe}pppA_{2′oMe}pC_{2′oMe}pN, m⁷G_{3′oMe}pppA_{2′oMe}pG_{2′oMe}pN, m⁷G_{3′oMe}pppA_{2′oMe}pU_{2′oMe}pN, m⁷G_{3′oMe}pppC_{2′oMe}pA_{2′oMe}pN, m⁷G_{3′oMe}pppC_{2′oMe}pC_{2′oMe}pN, m⁷G_{3′oMe}pppC_{2′oMe}pG_{2′oMe}pN, m⁷G_{3′oMe}pppC_{2′oMe}pU_{2′oMe}pN, m⁷G_{3′oMe}pppG_{2′oMep}A_{2′oMe}pN, m⁷G_{3′oMe}pppG_{2′oMe}pC_{2′oMe}pN, m⁷G_{3′oMe}pppG_{2′oMe}pG_{2′oMe}pN, m⁷G_{3′oMe}pppG_{2′oMe}pU_{2′oMe}pN, m⁷G_{3′oMe}pppU_{2′oMe}pA_{2′oMe}pN, m⁷G_{3′oMe}pppU_{2′oMe}pC_{2′oMe}pN, m⁷G_{3′oMe}pppU_{2′oMe}pG_{2′oMe}pN, and m⁷G_{3′oMe}pppU_{2′oMe}pU_{2′oMe}pN, where N is a natural, a modified, or an unnatural nucleoside base.

A tetranucleotide cap, in still other embodiments, comprises a sequence selected from the following sequences: m⁷GpppA_{2′oMe}pA_{2′oMe}pN, m⁷GpppA_{2′oMe}pC_{2′oMe}pN, m⁷GpppA_{2′oMe}pG_{2′oMe}pN, m⁷GpppA_{2′oMe}pU_{2′oMe}pN, m⁷GpppC_{2′oMe}pA_{2′oMe}pN, m⁷GpppC_{2′oMe}pC_{2′oMe}pN, m⁷GpppC_{2′oMe}pG_{2′oMe}pN, m⁷GpppC_{2′oMe}pU_{2′oMe}pN, m⁷GpppG_{2′oMep}A_{2′oMe}pN, m⁷GpppG_{2′oMe}pC_{2′oMe}pN, m⁷GpppG_{2′oMe}pG_{2′oMe}pN, m⁷GpppG_{2′oMe}pU_{2′oMe}pN, m⁷GpppU_{2′oMe}pA_{2′oMe}pN, m⁷GpppU_{2′oMe}pC_{2′oMe}pN, m⁷GpppU_{2′oMe}pG_{2′oMe}pN, and m⁷GpppU_{2′oMe}pU_{2′oMe}pN, where N is a natural, a modified, or an unnatural nucleoside base.

In some embodiments, a tetranucleotide cap comprises GGAG. In some embodiments, a tetranucleotide cap comprises the following structure:

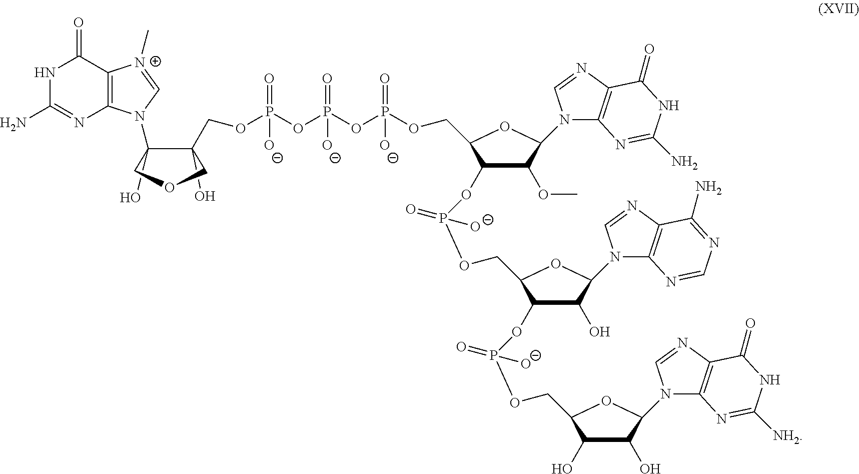

The capping efficiency of a post-transcriptional or co-transcriptional capping reaction may vary. The term “capping efficiency” may refer to the amount (e.g., expressed as a percentage) of mRNAs comprising a cap structure relative to the total mRNAs in a mixture (e.g., a post-translational capping reaction or a co-transcriptional calling reaction). In some embodiments, the capping efficiency of a capping reaction is at least 60%, 70%, 80%, 90%, 95%, 99%, or 99.9% (e.g., after the capping reaction at least 60%, 70%, 80%, 90%, 95%, 99%, or 99.9% of the input mRNAs comprise a cap). In some embodiments, multivalent co-IVT reactions do not affect the capping efficiency of the mRNAs resulting from the IVT reaction.

In vitro Transcription Methods Some aspects relate to methods of producing (e.g., synthesizing) a RNA transcript (e.g., mRNA transcript) comprising contacting a DNA template (e.g., a first input DNA and a second input DNA) with a RNA polymerase (e.g., a T7 RNA polymerase, a T7 RNA polymerase variant, etc.) under conditions that result in the production of the RNA transcript.

Some aspects relate to methods of performing an IVT reaction, comprising contacting a DNA template with the RNA polymerase (e.g., a T7 RNA polymerase, such as a T7 RNA polymerase variant) in the presence of nucleoside triphosphates and buffer under conditions that result in the production of RNA transcripts.

Other aspects provide co-transcriptional capping methods that comprise reacting a polynucleotide template with a T7 RNA polymerase variant, nucleoside triphosphates, and a cap analog under in vitro transcription reaction conditions to produce RNA transcript.

In some embodiments, a co-transcriptional capping method for RNA synthesis comprises reacting a polynucleotide template with (a) a T7 RNA polymerase variant comprising at least one amino acid substitution, relative to wild-type RNA polymerase (e.g., a T7 polymerase variant comprising amino acid substitutions at positions 437, 387, 350, and 351, relative to SEQ ID NO: 1), (b) nucleoside triphosphates, and (c) a cap analog (e.g., trinucleotide cap comprising sequence GpppA_{2′ome}pG), under in vitro transcription reaction conditions to produce RNA transcript, wherein the polynucleotide template includes a 2′-deoxythymidine residue at template position +1.

IVT conditions typically require a purified linear DNA template containing a promoter, nucleoside triphosphates, a buffer system that includes dithiothreitol (DTT) and magnesium ions, and an RNA polymerase. The exact conditions used in the transcription reaction depend on the amount of RNA needed for a specific application. Typical IVT reactions are performed by incubating a DNA template with an RNA polymerase and nucleoside triphosphates, including GTP, ATP, CTP, and UTP (or nucleotide analogs) in a transcription buffer. An RNA transcript having a 5′ terminal guanosine triphosphate is produced from this reaction.

The “percent identity,” “sequence identity,” “% identity,” or “% sequence identity” (as they may be interchangeably used herein) of two sequences (e.g., nucleic acid or amino acid) refers to a quantitative measurement of the similarity between two sequences (e.g., nucleic acid or amino acid). Percent identity can be determined using the algorithms of Karlin and Altschul, Proc. Natl. Acad. Sci. USA 87:2264-68, 1990, modified as in Karlin and Altschul, Proc. Natl. Acad. Sci. USA 90:5873-77, 1993. Such algorithms are incorporated into the NBLAST and XBLAST programs (version 2.0) of Altschul et al., J. Mol. Biol. 215:403-10, 1990. BLAST protein searches can be performed with the XBLAST program, score=50, word length=3, to obtain amino acid sequences homologous to the protein molecules of interest. Where gaps exist between two sequences, Gapped BLAST can be utilized as described in Altschul et al., Nucleic Acids Res. 25(17):3389-3402, 1997. When utilizing BLAST and Gapped BLAST programs, the default parameters of the respective programs (e.g., XBLAST and NBLAST) can be used. When a percent identity is stated, or a range thereof (e.g., at least, more than, etc.), unless otherwise specified, the endpoints shall be inclusive and the range (e.g., at least 70% identity) shall include all ranges within the cited range.

The input deoxyribonucleic acid (DNA) serves as a nucleic acid template for RNA polymerase. A DNA template may include a polynucleotide encoding a polypeptide of interest (e.g., an antigenic polypeptide). A DNA template, in some embodiments, includes a RNA polymerase promoter (e.g., a T7 RNA polymerase promoter) located 5′ from and operably linked to polynucleotide encoding a polypeptide of interest. A DNA template may also include a nucleotide sequence encoding a polyadenylation (polyA) tail located at the 3′ end of the gene of interest. In some embodiments, an input DNA comprises plasmid DNA (pDNA). The terms “plasmid DNA” or “pDNA” may refer to an extrachromosomal DNA molecule that is physically separated from chromosomal DNA in a cell and can replicate independently. In some embodiments, plasmid DNA is isolated from a cell (e.g., as a plasmid DNA preparation). In some embodiments, plasmid DNA comprises an origin of replication, which may contain one or more heterologous nucleic acids, for example nucleic acids encoding therapeutic proteins that may serve as a template for RNA polymerase. Plasmid DNA may be circularized or linear (e.g., plasmid DNA that has been linearized by a restriction enzyme digest).

In some embodiments, each input DNA (e.g., population of input DNA molecules) in a co-IVT reaction is obtained from a different source (e.g., synthesized separately, for example in different cells or populations of cells). In some embodiments, each input DNA (e.g., population of input DNA) is obtained from a different bacterial cell or population of bacterial cells. For example, in a co-IVT reaction having three populations of input DNAs, the first input DNA is produced in bacterial cell population A, the second input DNA is produced in bacterial cell population B, and the third input DNA is produced in bacterial population C, where each of A, B, and C are not the same bacterial culture (e.g., co-cultured in the same container or plate). In another example, two input DNAs obtained from different sources are i) chemically synthesized in separate synthesis reactions, or ii) produced by separate amplification (e.g., polymerase chain reactions (PCR reactions)). Methods of obtaining populations of input DNAs (e.g., plasmid DNAs) are known, for example as described by Sambrook, Joseph. Molecular Cloning: a Laboratory Manual. Cold Spring Harbor, N.Y. :Cold Spring Harbor Laboratory Press, 2001.

Some aspects comprise normalizing the amount of DNA used in the multivalent co-IVT reaction. In some embodiments, the normalization is based on the molar mass of the input DNAs. In some embodiments, the normalization is based on the degradation rate of the input DNAs. In some embodiments, the normalization is based on the degradation rate of the resultant mRNAs (e.g., measured based upon polyA variants present in the reaction mixture, or T7 polymerase abortive transcripts or truncated transcripts). In some embodiments, the normalization is based on the nucleotide content (e.g., amount of A, G, C, U, or any combination thereof) of the input DNAs. In some embodiments, the normalization is based on the purity of the input DNAs. In some embodiments the normalization is based on the polyA-tailing efficiency of the input DNAs. In some embodiments, the normalization is based on the lengths of the input DNAs.

In some embodiments, the normalization is based on the lowest level present in the input DNAs (e.g., lowest molar mass, degradation rate (e.g., of the input DNA and/or output RNA), nucleotide content, purity, and/or polyA-tailing efficiency). In some embodiments, the normalization is based on the highest level present in the input DNAs (e.g., highest molar mass, degradation rate (e.g., of the input DNA and/or output RNA), nucleotide context, purity, and/or polyA-tailing efficiency). In some embodiments, the normalization is based on the rate of RNA production of the input DNAs (e.g., the highest rate of RNA production of an input DNA or the lowest rate of RNA production of an input DNA in a reaction mixture).

Some aspects relate to IVT methods in which the amount of input DNA (e.g., a first DNA or second DNA) is adjusted or normalized in order to improve production of multivalent RNA compositions having a pre-defined mRNA ratio of components. The disclosure is based, in part, on the discovery that certain factors affecting multivalent RNA composition purity, such as large differences in size between input DNAs (e.g., a difference of more than 100, 200, 500, 1000, or more nucleotides in length) and/or polyA-tailing efficiency of a given DNA during IVT, may be addressed prior to the IVT by normalizing the amount of input DNA based upon one or more of those factors. For example, in some embodiments, the amount of two input DNAs is calculated based upon the desired molar ratio of the first RNA to the second RNA that are transcribed from the input DNAs. In some embodiments, the calculating comprises determining a plasmid mass ratio based upon the desired molar ratio of the input DNAs. In some embodiments, the amount of input DNAs is normalized based upon the highest polyA-tailing efficiency of the input DNAs during IVT.

The number of input DNAs (e.g., populations of input DNA molecules) used in an IVT reaction may vary, depending upon the number of different RNA molecules desired to be included in the multivalent RNA composition. In some embodiments, an IVT reaction mixture comprises 2 or more different input DNAs, for example 2, 3, 4, 5, 6, 7, 8, 9, 10, or more different input DNAs. In some embodiments, the IVT reaction comprises more than 10 different input DNAs. The term “different input DNAs” encompasses input DNAs that encode different RNAs, e.g., that have i) different lengths (whether or not the RNAs are identical over the entirety of the shorter of the two lengths), ii) different nucleotide sequences, iii) different chemical modification patterns, or iv) any combination of the foregoing.

The concentration of each of the populations of DNA molecules may also vary. In some embodiments, the concentration of each population of DNA molecules in an IVT reaction ranges from about 0.005 mg/mL to about 0.5 mg/ml. In some embodiments, the concentration of each population of DNA molecules in an IVT reaction ranges from about 0.02 mg/ml to about 0.05 mg/ml, 0.02 to about 0.15 mg/ml, about 0.05 mg/ml to about 0.20 mg/ml, about 0.175 to about 0.3 mg/ml, about 0.2 mg/ml to about 0.5 mg/ml, about 0.3 mg/ml to about 0.6 mg/ml, about 0.5 mg/ml to about 0.75 mg/ml, about 0.5 mg/ml to about 1.0 mg/ml, about 0.75 mg/ml to about 0.9 mg/ml, about 0.75 mg/ml to about 1.5 mg/ml, about 0.8 mg/ml to about 1.2 mg/ml, about 1.0 mg/ml to about 1.5 mg/ml, about 1.0 mg/ml to about 2.5 mg/ml, about 1.5 mg/ml to about 3.0 mg/ml, about 2.0 mg/ml to about 4.0 mg/ml, or about 2.5 mg/ml to about 5.0 mg/ml.

In some embodiments, the input DNAs are added to an IVT reaction are a predefined DNA ratio, which may comprise a ratio between 2, 3, 4, 5, 6, 7, 8, 9, 10, or more different input DNAs (e.g., depending on the number of different RNAs in a composition). In some embodiments, a pre-defined input DNA ratio comprises a ratio between more than 10 input DNAs. The term “pre-defined input DNA ratio” may refer to the desired final ratio of DNA molecules in an IVT reaction. The desired final ratio of input DNAs can depend upon the final peptide(s) or polypeptide product(s) encoded by RNAs encoded by the input DNAs. In some embodiments, the input DNAs can have a desired ratio that may comprise between 2 and 8 input DNAs (e.g., a:b, a:b:c, a:b:c:d, a:b:c:d:e, a:b:c:d:e:f, a:b:c:d:e:f:g, a:b:c:d:e:f:g:h, etc., where each of a-h is a number between 1 and 10). In some embodiments, the pre-defined input DNA ratio is different form the pre-defined mRNA ratio.

The size of two or more input DNAs (e.g., DNAs in two or more different populations of input DNAs) may vary. In some embodiments, an input DNA includes from about 15 to about 8,000 base pairs (e.g., from 15 to 50, 15 to 100, 15 to 200, 15 to 300, 15 to 400, 15 to 500, 15 to 600, 15 to 700, 15 to 800, 15 to 900, 15 to 1000, 15 to 1200, 15 to 1400, 15 to 1500, 15 to 1800, 15 to 2000, 15 to 2500, 15 to 3000, 50 to 100, 50 to 200, 50 to 300, 50 to 400, 50 to 500, 50 to 600, 50 to 700, 50 to 800, 50 to 900, 50 to 1000, 50 to 1200, 50 to 1400, 50 to 1500, 50 to 1800, 50 to 2000, 50 to 2500, 50 to 3000, 100 to 200, 100 to 300, 100 to 400, 100 to 500, 100 to 600, 100 to 700, 100 to 800, 100 to 900, 100 to 1000, 100 to 1200, 100 to 1400, 100 to 1500, 100 to 1800, 100 to 2000, 100 to 2500, 100 to 3000, 200 to 300, 200 to 400, 200 to 500, 200 to 600, 200 to 700, 200, to 800, 200 to 900, 200 to 1000, 200 to 1500, 200 to 3000, 500 to 1000, 500 to 1500, 500 to 2000, 500 to 2500, 500 to 3000, 1000 to 1500, 1000 to 2000, 1000 to 2500, 1000 to 3000, 1500 to 3000, 2500 to 3000, 2000 to 3000, 2500 to 4000, 3000 to 5000, 3500 to 6500, 5000 to 7500, or 6500 to 8000 base pairs.

The mass of each population of input DNA molecules in an IVT reaction may vary. In some embodiments, the mass of each population of input DNA ranges based upon the total volume of the IVT reaction mixture. In some embodiments, the mass of each population of each input DNA molecule in an IVT mixture individually varies from about 0.5% to about 99.9% of the total input DNA present in the IVT reaction mixture. In some embodiments, the molar ratio of each population of input DNA molecules in an IVT reaction may vary.

In some embodiments, two or more of the input DNA molecules used in an IVT reaction have a different length (e.g., comprises a different number of nucleotides). In some embodiments, the difference in length between two or more (e.g., 3, 4, 5, 6, 7, 8, 9, 10, or more) of the different input DNA molecules in an IVT reaction mixture is greater than 70 base pairs, 80 base pairs, 90 base pairs, or 100 base pairs (e.g., two input DNAs in a composition are not within 70, 80, 90, or 100 base pairs in length of one another). In some embodiments, the difference in length between two or more (e.g., 3, 4, 5, 6, 7, 8, 9, 10, or more) of the different input DNA molecules is more than 100 base pairs, for example 500 base pairs, 1000 base pairs, 1500 base pairs, 2000 base pairs, 3000 base pairs, 4000 base pairs, 5000 base pairs, 6000 base pairs, 7000 base pairs, 8000 base pairs, or more.

In some embodiments, two or more of the input DNA molecules used in an IVT reaction encode mRNA molecules that have a different length (e.g., comprises a different number of nucleotides). In some embodiments, the difference in length between two or more of the mRNA molecules encoded by different input DNA molecules in an IVT reaction mixture is greater than 70 nucleotides, 80 nucleotides, 90 nucleotides, or 100 nucleotides (e.g., two input DNAs in a composition encode mRNA molecules that are not are within 70, 80, 90, or 100 nucleotides in length of one another). In some embodiments, the difference in length between two or more of the mRNA molecules encoded by different input DNA molecules is more than 100 nucleotides, for example 500 nucleotides, 1000 nucleotides, 1500 nucleotides, 2000 nucleotides, 3000 nucleotides, 4000 nucleotides, or more.

In some embodiments, the multivalent IVT comprises co-transcription of at least 2 different input DNAs (e.g., at least 2 of DNA A, B, C, D, E, F, F, H, I, J, etc.) at a ratio of A:B:C:D:E:F:G:H:I:J, wherein if DNA A is normalized to 1, one or more of DNA B, C, D, E, F, G, H, I, J, etc. can each independently be present at an amount (e.g., a concentration) that is from 0.01 to 100 times the amount (e.g., a concentration) of A, such as from 0.05 times to 20 times the amount of A, 0.1 times to 10 times the amount of A, 0.2 times to 5 times the amount of A, 0.3 times to 3 times the amounts of A, 0.5 times to 2 times the amounts of A, 0.75 times to 1.4 times the amount of A, 0.8 times to 1.25 times the amount of A, or 0.9 times to 1.15 times the amount of A. One or more of DNA B, C, D, E, F, G, H, I, or J may also be absent.

In some embodiments, a multivalent RNA composition is produced by combining RNA transcripts (e.g., mRNAs) from separate sources. In some embodiments, a multivalent RNA composition is produced by separately transcribing two or more DNA templates in separate IVT reactions, and combining the transcribed RNAs. In some embodiments, an RNA transcript is produced by IVT, then added to one or more other RNAs. RNAs may be combined in any desired amount to produce a multivalent RNA composition comprising two or more RNAs in a specific ratio.

A RNA transcript, in some embodiments, is the product of an IVT reaction. A RNA transcript, in some embodiments, is a messenger RNA (mRNA) that includes a nucleotide sequence encoding a polypeptide of interest (e.g., a therapeutic protein or therapeutic peptide) linked to a polyA tail. In some embodiments, the mRNA is modified mRNA (mmRNA), which includes at least one modified nucleotide.

The nucleoside triphosphates (NTPs) may comprise unmodified or modified ATP, modified or unmodified UTP, modified or unmodified GTP, and/or modified or unmodified CTP. In some embodiments, NTPs of an IVT reaction comprise unmodified ATP. In some embodiments, NTPs of an IVT reaction comprise modified ATP. In some embodiments, NTPs of an IVT reaction comprise unmodified UTP. In some embodiments, NTPs of an IVT reaction comprise modified UTP. In some embodiments, NTPs of an IVT reaction comprise unmodified GTP. In some embodiments, NTPs of an IVT reaction comprise modified GTP. In some embodiments, NTPs of an IVT reaction comprise unmodified CTP. In some embodiments, NTPs of an IVT reaction comprise modified CTP.

The composition of NTPs in an IVT reaction may also vary. In some embodiments, each NTP in an IVT reaction is present in an equimolar amount. In some embodiments, each NTP in an IVT reaction is present in non-equimolar amounts. For example, ATP may be used in excess of GTP, CTP and UTP. As a non-limiting example, an IVT reaction may include 7.5 millimolar GTP, 7.5 millimolar CTP, 7.5 millimolar UTP, and 3.75 millimolar ATP. In some embodiments, the molar ratio of G:C:U:A is 2:1:0.5:1. In some embodiments, the molar ratio of G:C:U:A is 1:1:0.7:1. In some embodiments, the molar ratio of G:C:A:U is 1:1:1:1. The same IVT reaction may include 3.75 millimolar cap analog (e.g., trinucleotide cap or tetranucleotide cap). In some embodiments, the molar ratio of G:C:U:A:cap is 1:1:1:0.5:0.5. In some embodiments, the molar ratio of G:C:U:A:cap is 1:1:0.5:1:0.5. In some embodiments, the molar ratio of G:C:U:A:cap is 1:0.5:1:1:0.5. In some embodiments, the molar ratio of G:C:U:A:cap is 0.5:1:1:1:0.5. In some embodiments, the amount of NTPs in a co-IVT reaction is calculated empirically. For example, the rate of consumption for each NTP in an IVT reaction may be empirically determined for each individual input DNA, and then balanced ratios of NTPs based on those individual NTP consumption rates may be added to a co-IVT comprising multiple of the input DNAs.

In some embodiments, an IVT reaction mixture further comprises cap analog. The concentration of nucleoside triphosphates and cap analog present in an IVT reaction may vary. In some embodiments, NTPs and cap analog are present in the reaction at equimolar concentrations. In some embodiments, the molar ratio of cap analog (e.g., trinucleotide cap or tetranucleotide cap) to nucleoside triphosphates in the reaction is greater than 1:1. For example, the molar ratio of cap analog to nucleoside triphosphates in the reaction may be 2:1, 3:1, 4:1, 5:1, 6:1, 7:1, 8:1, 9:1, 10:1, 15:1, 20:1, 25:1, 50:1, or 100:1. In some embodiments, the molar ratio of cap analog (e.g., trinucleotide cap or tetranucleotide cap) to nucleoside triphosphates in the reaction is less than 1:1. For example, the molar ratio of cap analog (e.g., trinucleotide cap or tetranucleotide cap) to nucleoside triphosphates in the reaction may be 1:2, 1:3, 1:4, 1:5, 1:6, 1:7, 1:8, 1:9, 1:10, 1:15, 1:20, 1:25, 1:50, or 1:100.

In some embodiments, a RNA transcript (e.g., mRNA transcript) includes a modified nucleobase selected from pseudouridine (ψ), 1-methylpseudouridine (m¹ψ), 5-methoxyuridine (mo⁵U), 5-methylcytidine (m⁵C), α-thio-guanosine and α-thio-adenosine. In some embodiments, a RNA transcript (e.g., mRNA transcript) includes a combination of at least two (e.g., 2, 3, 4 or more) of the foregoing modified nucleobases.

In some embodiments, a RNA transcript (e.g., mRNA transcript) includes pseudouridine (ψ). In some embodiments, a RNA transcript (e.g., mRNA transcript) includes 1-methylpseudouridine (m¹ψ). In some embodiments, a RNA transcript (e.g., mRNA transcript) includes 5-methoxyuridine (mo⁵U). In some embodiments, a RNA transcript (e.g., mRNA transcript) includes 5-methylcytidine (m⁵C). In some embodiments, a RNA transcript (e.g., mRNA transcript) includes α-thio-guanosine. In some embodiments, a RNA transcript (e.g., mRNA transcript) includes α-thio-adenosine.

In some embodiments, the polynucleotide (e.g., RNA polynucleotide, such as mRNA polynucleotide) is uniformly modified (e.g., fully modified, modified throughout the entire sequence) for a particular modification. For example, a polynucleotide can be uniformly modified with 1-methylpseudouridine (m¹ψ), meaning that all uridine residues in the mRNA sequence are replaced with 1-methylpseudouridine (m¹ψ). Similarly, a polynucleotide can be uniformly modified for any type of nucleoside residue present in the sequence by replacement with a modified residue such as any of those set forth above. Alternatively, the polynucleotide (e.g., RNA polynucleotide, such as mRNA polynucleotide) may not be uniformly modified (e.g., partially modified, part of the sequence is modified). Each possibility represents a separate embodiment.

The buffer system of an IVT reaction mixture may vary. In some embodiments, the buffer system contains tris. The concentration of tris used in an IVT reaction, for example, may be at least 10 mM, at least 20 mM, at least 30 mM, at least 40 mM, at least 50 mM, at least 60 mM, at least 70 mM, at least 80 mM, at least 90 mM, at least 100 mM or at least 110 mM phosphate. In some embodiments, the concentration of phosphate is 20-60 mM or 10-100 mM.

In some embodiments, the buffer system contains dithiothreitol (DTT). The concentration of DTT used in an IVT reaction, for example, may be at least 1 mM, at least 5 mM, or at least 50 mM. In some embodiments, the concentration of DTT used in an IVT reaction is 1-50 mM or 5-50 mM. In some embodiments, the concentration of DTT used in an IVT reaction is 5 mM.

In some embodiments, the buffer system contains magnesium. In some embodiments, the molar ratio of NTP to magnesium ions (Mg²⁺; e.g., MgCl2) present in an IVT reaction is 1:1 to 1:5. For example, the molar ratio of NTP to magnesium ions may be 1:0.25, 1:0.5, 1:1, 1:2, 1:3, 1:4 or 1:5.

In some embodiments, the molar ratio of NTP plus cap analog (e.g., trinucleotide cap, such as GAG) to magnesium ions (Mg²⁺; e.g., MgCl₂) present in an IVT reaction is 1:1 to 1:5. For example, the molar ratio of NTP+trinucleotide cap (e.g., GAG) to magnesium ions may be 1:1, 1:2, 1:3, 1:4 or 1:5.

In some embodiments, the buffer system contains Tris-HCl, spermidine (e.g., at a concentration of 1-30 mM), TRITON® X-100 (polyethylene glycol p-(1,1,3,3-tetramethylbutyl)-phenyl ether) and/or polyethylene glycol (PEG).

In some embodiments, IVT methods further comprise a step of separating (e.g., purifying) in vitro transcription products (e.g., mRNA) from other reaction components. In some embodiments, the separating comprises performing chromatography on the IVT reaction mixture. In some embodiments, the chromatography comprises size-based (e.g., length-based) chromatography. In some embodiments, the chromatography comprises oligo-dT chromatography.

The addition of nucleoside triphosphates (NTPs) to the 3′ end of a growing RNA strand is catalyzed by a polymerase, such as T7 RNA polymerase, for example, a T7 RNA polymerase variant (e.g., RNA polymerase comprising D653W/E350W/D351V substitutions). In some embodiments, the RNA polymerase (e.g., T7 RNA polymerase variant) is present in a reaction (e.g., an IVT reaction) at a concentration of 0.01 mg/ml to 1 mg/ml. For example, the RNA polymerase may be present in a reaction at a concentration of 0.01 mg/mL, 0.05 mg/ml, 0.1 mg/ml, 0.5 mg/ml or 1.0 mg/ml.

Surprisingly, use of the combination of a T7 RNA polymerase variant (e.g., RNA polymerase comprising D653W/E350W/D351V substitutions) with a cap analog (e.g., GpppA_{2′ome}pG), in an in vitro transcription reaction, for example, results in the production of RNA transcript, wherein greater than 80% of the RNA transcript produced includes a functional cap. In some embodiments, greater than 85% of the RNA transcript produced includes a functional cap. In some embodiments, greater than 90% of the RNA transcript produced includes a functional cap. In some embodiments, greater than 95% of the RNA transcript produced includes a functional cap. In some embodiments, greater than 96% of the RNA transcript produced includes a functional cap. In some embodiments, greater than 97% of the RNA transcript produced includes a functional cap. In some embodiments, greater than 98% of the RNA transcript produced includes a functional cap. In some embodiments, greater than 99% of the RNA transcript produced includes a functional cap.

Also surprising was the finding that use of a polynucleotide template that includes a 2′-deoxythymidine residue or 2′-deoxycytidine residue at template position +1 results in the production of RNA transcript, wherein greater than 80% (e.g., greater than 85%, greater than 90%, or greater than 95%) of the RNA transcript produced includes a functional cap. Thus, in some embodiments, a polynucleotide (e.g., DNA) template used, for example, in an IVT reaction, includes a 2′-deoxythymidine residue at template position +1. In other embodiments, a polynucleotide (e.g., DNA) template used, for example, in an IVT reaction, includes a 2′-deoxycytidine residue at template position +1.

### Applications

The RNA transcripts produced using an RNA polymerase variant may include mRNA (including modified mRNA and/or unmodified RNA), lncRNA, self-replicating RNA, circular RNA, CRISPR guide RNA, and the like. In embodiments, the RNA is RNA (e.g., mRNA or self-replicating RNA) that encodes a polypeptide (e.g., a therapeutic polypeptide). Thus, the RNA transcripts produced using RNA polymerase variants may be used in a myriad of applications.

For example, the RNA transcripts may be used to produce polypeptides of interest, e.g., therapeutic proteins, vaccine antigen, and the like. In some embodiments, the RNA transcripts are therapeutic RNAs. A therapeutic mRNA is an mRNA that encodes a therapeutic protein (the term ‘protein’ encompasses peptides). Therapeutic proteins mediate a variety of effects in a host cell or in a subject to treat a disease or ameliorate the signs and symptoms of a disease. For example, a therapeutic protein can replace a protein that is deficient or abnormal, augment the function of an endogenous protein, provide a novel function to a cell (e.g., inhibit or activate an endogenous cellular activity, or act as a delivery agent for another therapeutic compound (e.g., an antibody-drug conjugate). Therapeutic mRNA may be useful for the treatment of the following diseases and conditions: bacterial infections, viral infections, parasitic infections, cell proliferation disorders, genetic disorders, and autoimmune disorders. Other diseases and conditions are encompassed herein.

An RNA transcript produced using an RNA polymerase variant may encode one or more biologics. A biologic is a polypeptide-based molecule that may be used to treat, cure, mitigate, prevent, or diagnose a serious or life-threatening disease or medical condition. Biologics include, but are not limited to, allergenic extracts (e.g. for allergy shots and tests), blood components, gene therapy products, human tissue or cellular products used in transplantation, vaccines, monoclonal antibodies, cytokines, growth factors, enzymes, thrombolytics, and immunomodulators, among others.

One or more biologics currently being marketed or in development may be encoded by the RNA produced by an RNA polymerase variant. While not wishing to be bound by theory, it is believed that incorporation of the encoding polynucleotides of a known biologic into the RNA will result in improved therapeutic efficacy due at least in part to the specificity, purity and/or selectivity of the construct designs.

An RNA transcript produced using an RNA polymerase variant may encode one or more antibodies. The term “antibody” includes monoclonal antibodies (including full length antibodies which have an immunoglobulin Fc region), antibody compositions with polyepitopic specificity, multispecific antibodies (e.g., bispecific antibodies, diabodies, and single-chain molecules), as well as antibody fragments. The term “immunoglobulin” (Ig) is used interchangeably with “antibody” herein. A monoclonal antibody is an antibody obtained from a population of substantially homogeneous antibodies, i.e., the individual antibodies comprising the population are identical except for possible naturally occurring mutations and/or post-translation modifications (e.g., isomerizations, amidations) that may be present in minor amounts. Monoclonal antibodies are highly specific, being directed against a single antigenic site.

Monoclonal antibodies specifically include chimeric antibodies (immunoglobulins) in which a portion of the heavy and/or light chain is identical with or homologous to corresponding sequences in antibodies derived from a particular species or belonging to a particular antibody class or subclass, while the remainder of the chain(s) is(are) identical with or homologous to corresponding sequences in antibodies derived from another species or belonging to another antibody class or subclass, as well as fragments of such antibodies, so long as they exhibit the desired biological activity. Chimeric antibodies include, but are not limited to, “primatized” antibodies comprising variable domain antigen-binding sequences derived from a non-human primate (e.g., Old World Monkey, Ape etc.) and human constant region sequences.

An RNA transcript produced using an RNA polymerase variant may encode one or more vaccine antigens. A vaccine antigen is a biological preparation that improves immunity to a particular disease or infectious agent. One or more vaccine antigens currently being marketed or in development may be encoded by the RNA. Vaccine antigens encoded in the RNA may be utilized to treat conditions or diseases in many therapeutic areas such as, but not limited to, cancer, allergy and infectious disease. In some embodiments, a cancer vaccine may be a personalized cancer vaccine in the form of a concatemer or individual RNAs encoding peptide epitopes or a combination thereof.

An RNA transcript produced using an RNA polymerase variant may be designed to encode on or more antimicrobial peptides (AMP) or antiviral peptides (AVP). AMPs and AVPs have been isolated and described from a wide range of animals such as, but not limited to, microorganisms, invertebrates, plants, amphibians, birds, fish, and mammals.

In some embodiments, RNA transcripts are used as radiolabeled RNA probes. In some embodiments, RNA transcripts are used for non-isotopic RNA labeling. In some embodiments, RNA transcripts are used as guide RNA (gRNA) for gene targeting. In some embodiments, RNA transcripts (e.g., mRNA) are used for in vitro translation and micro injection. In some embodiments, RNA transcripts are used for RNA structure, processing and catalysis studies. In some embodiments, RNA transcripts are used for RNA amplification. In some embodiments, RNA transcripts are used as anti-sense RNA for gene expression experiment.

Wild-type T7 RNA Polymerase
(SEQ ID NO: 1)
MNTINIAKNDFSDIELAAIPFNTLADHYGERLAREQLALEHESYEMGEARFRKMFERQLKAGEVADNAAAKPLITTL
LPKMIARINDWFEEVKAKRGKRPTAFQFLQEIKPEAVAYITIKTTLACLTSADNTTVQAVASAIGRAIEDEARFGRI
RDLEAKHFKKNVEEQLNKRVGHVYKKAFMQVVEADMLSKGLLGGEAWSSWHKEDSIHVGVRCIEMLIESTGMVSLHR
QNAGVVGQDSETIELAPEYAEAIATRAGALAGISPMFQPCVVPPKPWTGITGGGYWANGRRPLALVRTHSKKALMRY
EDVYMPEVYKAINIAQNTAWKINKKVLAVANVITKWKHCPVEDIPAIEREELPMKPEDIDMNPEALTAWKRAAAAVY
RKDKARKSRRISLEFMLEQANKFANHKAIWFPYNMDWRGRVYAVSMFNPQGNDMTKGLLTLAKGKPIGKEGYYWLKI
HGANCAGVDKVPFPERIKFIEENHENIMACAKSPLENTWWAEQDSPFCFLAFCFEYAGVQHHGLSYNCSLPLAFDGS
CSGIQHFSAMLRDEVGGRAVNLLPSETVQDIYGIVAKKVNEILQADAINGTDNEVVTVTDENTGEISEKVKLGTKAL
AGQWLAYGVTRSVTKRSVMTLAYGSKEFGFRQQVLEDTIQPAIDSGKGLMFTQPNQAAGYMAKLIWESVSVTVVAAV
EAMNWLKSAAKLLAAEVKDKKTGEILRKRCAVHWVTPDGFPVWQEYKKPIQTRLNLMFLGQFRLQPTINTNKDSEID
AHKQESGIAPNFVHSQDGSHLRKTVVWAHEKYGIESFALIHDSFGTIPADAANLFKAVRETMVDTYESCDVLADFYD
QFADQLHESQLDKMPALPAKGNLNLRDILESDFAFA

### ADDITIONAL EMBODIMENTS

Additional embodiments are encompassed by the following numbered paragraphs 1-71:

1. A ribonucleic acid (RNA) polymerase variant comprising: an amino acid sequence comprising (i) an amino acid substitution at position E350, (ii) an amino acid substitution at position D351, and (iii) an amino acid substitution at position K387, position N437, or at position K387 and position N437, relative to a wild-type T7 RNA polymerase comprising the amino acid sequence of SEQ ID NO: 1.
2. The RNA polymerase variant of paragraph 1, wherein the amino acid sequence of the variant comprises an amino acid substitution at position K387.
3. The RNA polymerase variant of paragraph 1, wherein the amino acid sequence of the variant comprises an amino acid substitution at position N437.
4. The RNA polymerase variant of paragraph 1, wherein the amino acid sequence of the variant comprises an amino acid substitution at position K387 and at position N437.
5. The RNA polymerase variant of any one of paragraphs 1-4, wherein the amino acid substitution at position K387 is a polar, neutral amino acid.
6. The RNA polymerase variant of paragraph 5, wherein the polar, neutral amino acid is selected from asparagine (N), cysteine (C), glutamine (Q), methionine (M), serine (S), and threonine (T).
7. The RNA polymerase variant of paragraph 6, wherein the polar, neutral amino acid is asparagine (K387N).
8. The RNA polymerase variant of paragraph 6, wherein the polar, neutral amino acid is cysteine (K387C).
9. The RNA polymerase variant of paragraph 6, wherein the polar, neutral amino acid is glutamine (K387Q).
10. The RNA polymerase variant of paragraph 6, wherein the polar, neutral amino acid is methionine (K387M).
11. The RNA polymerase variant of paragraph 6, wherein the polar, neutral amino acid is serine (K387S).
12. The RNA polymerase variant of paragraph 6, wherein the polar, neutral amino acid is threonine (K387T).
13. The RNA polymerase variant of any one of paragraphs 1-12, wherein the amino acid substitution at position N437 is an aromatic amino acid.
14. The RNA polymerase variant of paragraph 13, wherein the aromatic amino acid is selected from tryptophan (W), tyrosine (Y), and phenylalanine (F).
15. The RNA polymerase variant of paragraph 14, wherein the aromatic amino acid is tryptophan (N437W).
16. The RNA polymerase variant of paragraph 14, wherein the aromatic amino acid is tyrosine (N437Y).
17. The RNA polymerase variant of paragraph 14, wherein the aromatic amino acid is phenylalanine (N437F).
18. A ribonucleic acid (RNA) polymerase variant comprising an amino acid sequence that comprises (i) an amino acid substitution at position E350, (ii) an amino acid substitution at D351, and (iii) an amino acid substitution at position D653, relative to a wild-type T7 RNA polymerase comprising the amino acid sequence of SEQ ID NO: 1.
19. The RNA polymerase variant of paragraph 18, wherein the amino acid substitution at position D653 is an aromatic amino acid.
20. The RNA polymerase variant of paragraph 19, wherein the aromatic amino acid is selected from tryptophan (W), tyrosine (Y), and phenylalanine (F).
21. The RNA polymerase variant of paragraph 20, wherein the aromatic amino acid is tryptophan (D653W).
22. The RNA polymerase variant of paragraph 20, wherein the aromatic amino acid is tyrosine (D653Y).
23. The RNA polymerase variant of paragraph 20, wherein the aromatic amino acid is phenylalanine (D653F).
24. The RNA polymerase variant of any one of paragraphs 1-23, wherein the amino acid substitution at position E350 is an aromatic amino acid.
25. The RNA polymerase variant of paragraph 24, wherein the aromatic amino acid is selected from tryptophan (W), tyrosine (Y), and phenylalanine (F).
26. The RNA polymerase variant of paragraph 25, wherein the aromatic amino acid is tryptophan (E350W).
27. The RNA polymerase variant of paragraph 25, wherein the aromatic amino acid is tyrosine (E350Y).
28. The RNA polymerase variant of paragraph 25, wherein the aromatic amino acid is phenylalanine (E350F).
29. The RNA polymerase variant of paragraphs 1-28, wherein the amino acid substitution at position D351 is a non-polar, aliphatic amino acid.
30. The RNA polymerase variant of paragraph 29, wherein the non-polar, aliphatic amino acid is selected from alanine (A), glycine (G), isoleucine (I), leucine (L), proline (P), and valine (V).
31. The RNA polymerase variant of paragraph 30, wherein the non-polar, aliphatic amino acid is alanine (D351A).
32. The RNA polymerase variant of paragraph 30, wherein the non-polar, aliphatic amino acid is glycine (D351G).
33. The RNA polymerase variant of paragraph 30, wherein the non-polar, aliphatic amino acid is isoleucine (D351I).
34. The RNA polymerase variant of paragraph 30, wherein the non-polar, aliphatic amino acid is leucine (D351L).
35. The RNA polymerase variant of paragraph 30, wherein the non-polar, aliphatic amino acid is proline (D351P).
36. The RNA polymerase variant of paragraph 30, wherein the non-polar, aliphatic amino acid is valine (D351V).
37. An RNA polymerase variant comprising: an amino acid sequence having at least 70% identity to the amino acid sequence of SEQ ID NO: 1, wherein the amino acid sequence of the variant comprises (i) an amino acid substitution at position E350, (ii) an amino acid substitution at D351, and (iii) an amino acid substitution at position K387, position N437, or at position K387 and position N437, relative to a wild-type T7 RNA polymerase comprising the amino acid sequence of SEQ ID NO: 1.
38. The RNA polymerase variant of paragraph 37, wherein the amino acid sequence has at least 75%, at least 80%, at least 85%, at least 95%, or at least 98% identity to the amino acid sequence of SEQ ID NO: 1.
39. The RNA polymerase variant of paragraph 37 or 38, wherein the amino acid sequence of the variant comprises an amino acid substitution at position K387.
40. The RNA polymerase variant of paragraph 37 or 38, wherein the amino acid sequence of the variant comprises an amino acid substitution at position N437.
41. The RNA polymerase variant of paragraph 37 or 38, wherein the amino acid sequence of the variant comprises an amino acid substitution at position K387 and at position N437.
42. The RNA polymerase variant of any one of paragraphs 37-41, wherein the amino acid substitution at position K387 is a polar, neutral amino acid.
43. The RNA polymerase variant of paragraph 42, wherein the polar, neutral amino acid is selected from asparagine (K387N), cysteine (K387C), glutamine (K387Q), methionine (K387M), serine (K387S), and threonine (K387T).
44. The RNA polymerase variant of any one of paragraphs 37-42, wherein the amino acid substitution at position N437 is an aromatic amino acid.
45. The RNA polymerase variant of paragraph 44, wherein the aromatic amino acid is selected from tryptophan (N437W), tyrosine (N437Y), and phenylalanine (N437F).
46. An RNA polymerase variant comprising: an amino acid sequence having at least 70% identity to the amino acid sequence of SEQ ID NO: 1, wherein the amino acid sequence of the variant comprises (i) an amino acid substitution at position E350, (ii) an amino acid substitution at D351, and (iii) an amino acid substitution at position D653, relative to a wild-type T7 RNA polymerase comprising the amino acid sequence of SEQ ID NO: 1.
47. The RNA polymerase variant of paragraph 46, wherein the amino acid sequence has at least 75%, at least 80%, at least 85%, at least 95%, or at least 98% identity to the amino acid sequence of SEQ ID NO: 1.
48. The RNA polymerase variant of paragraph 46 or 47, wherein the amino acid substitution at position D653 is an aromatic amino acid.
49. The RNA polymerase variant of paragraph 48, wherein the aromatic amino acid is selected from tryptophan (D653W), tyrosine (D653Y), and phenylalanine (D653F).
50. The RNA polymerase variant of any one of paragraphs 37-49, wherein the amino acid substitution at position E350 is an aromatic amino acid.
51. The RNA polymerase variant of paragraph 50, wherein the aromatic amino acid is selected from tryptophan (E350W), tyrosine (E350Y), and phenylalanine (E350F).
52. The RNA polymerase variant of any one of paragraphs 37-51, wherein the amino acid substitution at position D351 is a non-polar, aliphatic amino acid.
53. The RNA polymerase variant of paragraph 52, wherein the non-polar, aliphatic amino acid is selected from alanine (D351A), glycine (D351G), isoleucine (D351I), leucine (D351L), proline (D351P), and valine (D351V).
54. A ribonucleic acid (RNA) polymerase variant comprising the amino acid sequence of SEQ ID NO: 2, wherein X¹ is an aromatic amino acid, optionally selected from W, Y, and F; X² is selected from a non-polar, aliphatic amino acid, optionally selected from A, G, I, L, P, and V; X³ is a polar, neutral amino acid, optionally selected from N, C, Q, M, S, and T; and X⁴ is an aromatic amino acid, optionally selected from W, Y, and F.
55. A ribonucleic acid (RNA) polymerase variant comprising the amino acid sequence of SEQ ID NO: 6.
56. A ribonucleic acid (RNA) polymerase variant comprising the amino acid sequence of SEQ ID NO: 3, wherein X¹ is an aromatic amino acid, optionally selected from W, Y, and F; X² is selected from a non-polar, aliphatic amino acid, optionally selected from A, G, I, L, P, and V; and X⁴ is an aromatic amino acid, optionally selected from W, Y, and F.
57. A ribonucleic acid (RNA) polymerase variant comprising the amino acid sequence of SEQ ID NO: 7.
58. A ribonucleic acid (RNA) polymerase variant comprising the amino acid sequence of SEQ ID NO: 4, wherein X¹ is an aromatic amino acid, optionally selected from W, Y, and F; X² is selected from a non-polar, aliphatic amino acid, optionally selected from A, G, I, L, P, and V; and X³ is a polar, neutral amino acid, optionally selected from N, C, Q, M, S, and T.
59. A ribonucleic acid (RNA) polymerase variant comprising the amino acid sequence of SEQ ID NO: 8.
60. A ribonucleic acid (RNA) polymerase variant comprising the amino acid sequence of SEQ ID NO: 5, wherein X¹ is an aromatic amino acid, optionally selected from W, Y, and F; X² is selected from a non-polar, aliphatic amino acid, optionally selected from A, G, I, L, P, and V; and X⁵ is an aromatic amino acid, optionally selected from W, Y, and F.
61. A ribonucleic acid (RNA) polymerase variant comprising the amino acid sequence of SEQ ID NO: 9.
62. A method comprising: producing a messenger RNA (mRNA) in an in vitro transcription reaction that comprises a DNA, nucleoside triphosphates, the RNA polymerase variant of any one of paragraphs 1-53, and optionally a cap analog.
63. The method of paragraph 62, wherein the reaction comprises the cap analog.
64. The method of paragraph 62 or 63, wherein the cap analog is a dinucleotide cap analog, a trinucleotide cap analog, or a tetranucleotide cap analog.
65. The method of paragraph 64, wherein the cap analog is a trinucleotide cap analog comprising a GAG sequence.
66. The method of paragraph 65, wherein the GAG cap analog comprises a compound selected from:

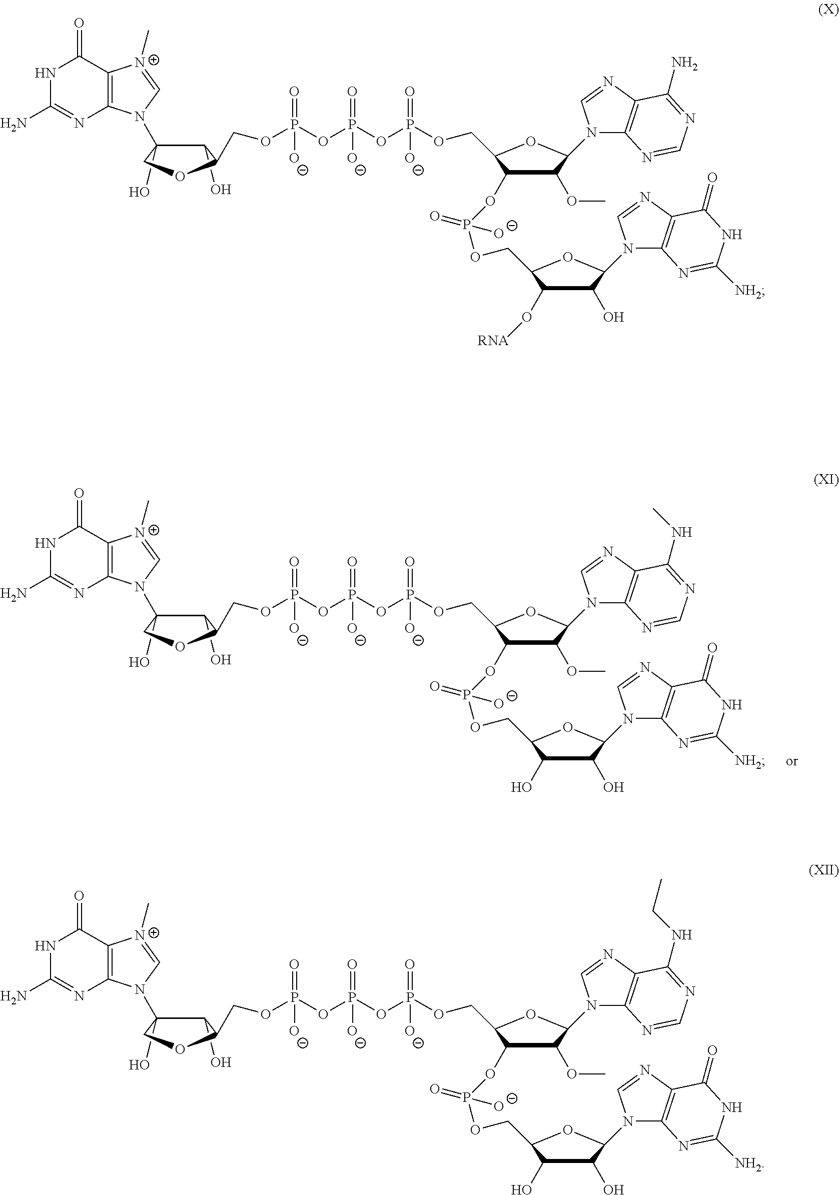

67. The method of paragraph 64, wherein the tetranucleotide cap analog comprises a GGAG sequence.
68. The method of paragraph 67, wherein the tetranucleotide cap analog comprises a compound selected from:

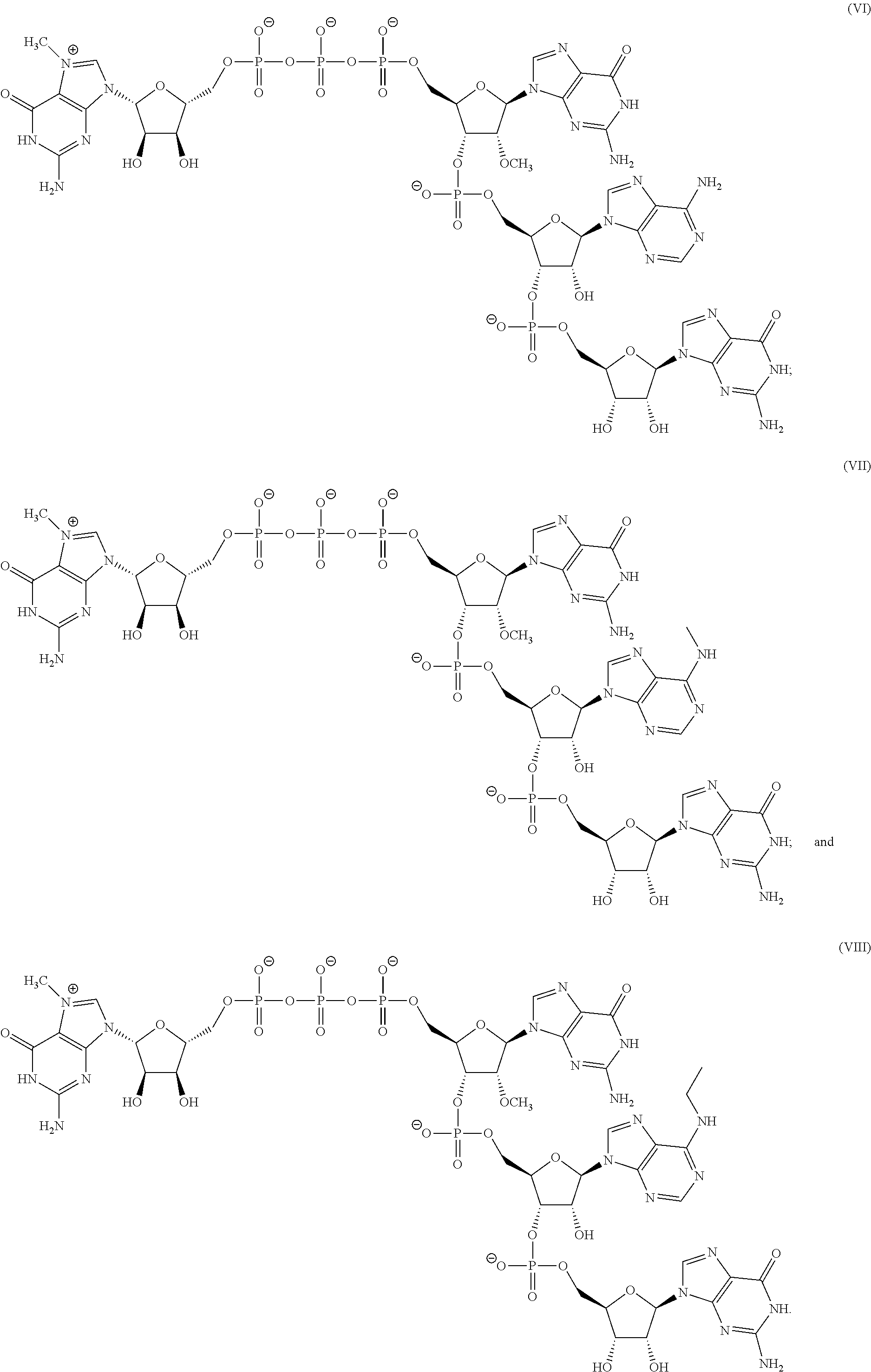

69. The method of any one of the paragraphs 62-68, wherein the DNA includes a 2′-deoxythymidine residue or a 2′-deoxycytidine residue at position +1.
70. A composition or kit comprising the RNA polymerase variant of any one of paragraphs 1-61 and an in vitro transcription (IVT) reagent selected from the group consisting of a DNA, nucleoside triphosphates, and a cap analog.
71. A nucleic acid encoding the RNA polymerase variant of any one of paragraphs 1-61.

### EXAMPLES

### Example 1. IVT Reactions Using RNA Polymerase Variants

In vitro transcription (IVT) reactions were performed using DNA template, GGAG cap analog, and selected individual RNA polymerase variants as provided in Table 1. Specifically, RNA polymerase variants comprising N437Y, K387S, E350W, and D351V substitutions (SEQ ID NO: 6; “Variant A”); N437Y, E350W, and D351V substitutions (SEQ ID NO: 7; “Variant B”); K387S, E350W, and D351V substitutions (SEQ ID NO: 8; “Variant C”); and D653W, E350W, and D351V substitutions (SEQ ID NO: 9; “Variant D”) were tested in this Example. Reactions were also performed using control T7 RNA polymerase (SEQ ID NO: 1). Following IVT reactions, transcribed RNA products from each reaction were characterized to address the quality of said RNA products, including total RNA yield, capping efficiency (percentage of total RNA comprising a GGAG cap), dsRNA contamination, and tail purity.

The overall yields of total RNA, following an oligo dT purification, were measured by UV absorption. The total RNA products were analyzed by LC-MS to determine capping efficiency (i.e., percent of transcribed RNA comprising a GGAG cap). A standard ELISA was used to assess dsRNA contaminants (e.g., dsRNA longer than 40 nucleotide base pairs) following IVT reactions in this Example. A Tris RP (reverse-phase) method was used to assess percent tailed RNA (i.e., percent of transcribed RNA comprising a polyA tail).

Each of the tested RNA polymerase variants generated RNA in IVT reactions with at least 80% capped RNA (percentage of total RNA comprising a GGAG cap) and at least ˜80% tailed RNA (i.e., percent of transcribed RNA comprising a polyA tail). RNA polymerase variants comprising N437Y, E350W, and D351V substitutions (SEQ ID NO: 7); and K387S, E350W, and D351V substitutions (SEQ ID NO: 8) generated less than 0.007% dsRNA (w:w) Further, the yields of total RNA for each of the tested RNA polymerase variants (greater than 8 mg/mL) was comparable to control T7 RNA polymerase.

Each of the tested RNA polymerase variants performed comparably or better than the control T7 RNA polymerase across each of the tested characteristics. Specifically, N437Y+K387S+E350W+D351V provided RNA with higher capping efficiency (˜85% capped RNA), similar yield and similar tailed purity relative to control T7 RNA polymerase. N437Y+E350W +D351V provided RNA with higher capping efficiency (˜80% capped RNA), similar yield, similar tailed purity, and similar dsRNA contamination relative to control T7 RNA polymerase. K387S+E350W+D351V provided RNA with higher capping efficiency (˜83% capped RNA), similar yield, higher tailed purity (˜85% tailed RNA), and less dsRNA contamination (0.00327 dsRNA wt:wt) relative to control T7 RNA polymerase. D653W+E350W+D351V provided RNA with higher capping efficiency (˜95% capped RNA) relative to control T7 RNA polymerase.

Data for each of tested RNA polymerases is provided in Table 3 and FIGS. 1A-1D.

TABLE 3
Characteristics of RNA produced by
RNA polymerases used in Example 1

| | | | | Percent |
|---|---|---|---|---|
| | Yield | Percent | Percent | dsRNA |
| | (mg/ml) | Capping | tailed | (w:w) |
| Control RNAP | 9.34 | 69.51 | 83.17 | 0.00624 |
| N437Y + K387S + E350W + | 11.14 | 84.95 | 79.51 | 0.01583 |
| D351V | | | | |
| N437Y + E350W + D351V | 9.56 | 80.19 | 80.87 | 0.006955 |
| K387S + E350W + D351V | 9.20 | 82.88 | 85.00 | 0.00327 |
| D653W + E350W + D351V | 8.87 | 94.47 | 80.30 | 0.02815 |

### Example 2. RNA Polymerase Variants Produce RNA Products with High Levels of Capping Efficiency at Low Concentrations of GGAG Cap Analog

In vitro transcription reactions were performed using DNA template, equimolar NTPs, a variable amount of GGAG tetranucleotide cap analog (0.25 mM, 0.5 mM, 0.75 mM, 1 mM, 1.25 mM, 1.5 mM, 3 mM) and T7 RNA polymerase. RNA polymerase variants comprising N437Y, K387S, E350W, and D351V substitutions (SEQ ID NO: 6); N437Y, E350W, and D351V substitutions (SEQ ID NO: 7); K387S, E350W, and D351V substitutions (SEQ ID NO: 8); and D653W, E350W, and D351V substitutions (SEQ ID NO: 9) were tested in this Example. Reactions were also performed using control T7 RNA polymerase.

Following the IVT reaction, mRNA products were oligo-dT purified before being analyzed by LC-MS to determine the % capped RNA (i.e., percent of transcribed RNA comprising a cap), by HPLC to determine the RNA yield of the reaction, and by Tris RP (reverse-phase) method to determine percent tailed RNA.

Each of the tested RNA polymerase variants produced RNA with percent capped RNA at higher levels than the control polymerase variant in the presence of GGAG cap analog, regardless of the concentration of the GGAG analog (FIG. 2A). Even at the lowest tested concentrations of GGAG cap analog (0.25 mM), all of tested variants produced at least 50% capped RNA, considerably higher than the ˜25% capped RNA produced by the control polymerase variant. At 1.5 mM GGAG cap analog, all of tested variants produced about 80-95% capped RNA.

Each of the tested variants produced RNA with comparable yield (FIG. 2B) and percent tailed RNA (FIG. 2C) relative to the control polymerase variant.

These data demonstrate that each of tested RNA polymerase variants (i.e., N437Y+K387S+E350W+D351V; N437Y+E350W+D351V; K387S+E350W+D351V; and D653W+E350W+D351V) are capable of producing RNA with higher capping efficiency than control T7 RNA polymerase without giving up any yield or tailed content.

### Equivalents and Scope

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1. A ribonucleic acid (RNA) polymerase variant comprising an amino acid sequence having at least 90% sequence identity to the amino acid sequence of any one of SEQ ID NOs: 2-9, wherein the amino acid sequence comprises an amino acid substitution at position D351 and at least two additional amino acid substitutions, relative to a RNA polymerase comprising the amino acid sequence of SEQ ID NO: 1.

2. The RNA polymerase variant of claim 1, comprising an amino acid sequence having at least 95% sequence identity to the amino acid sequence of any one of SEQ ID NOs: 2-9.

3. The RNA polymerase variant of claim 1, comprising an amino acid sequence comprising the amino acid sequence of any one of SEQ ID NOs: 2-9.

4. The RNA polymerase variant of claim 1, comprising at least one, at least two, at least three, or at least four amino acid substitutions, relative to a wild-type T7 RNA polymerase comprising the amino acid sequence of SEQ ID NO: 1.

5. A ribonucleic acid (RNA) polymerase variant comprising the amino acid sequence of SEQ ID NO: 6.

6. A ribonucleic acid (RNA) polymerase variant comprising the amino acid sequence of SEQ ID NO: 7.

7. A ribonucleic acid (RNA) polymerase variant comprising the amino acid sequence of SEQ ID NO: 8.

8. A ribonucleic acid (RNA) polymerase variant comprising the amino acid sequence of SEQ ID NO: 9.

9. A method comprising: producing a messenger RNA (mRNA) in an in vitro transcription reaction that comprises a DNA, nucleoside triphosphates, the RNA polymerase variant of claim 1.

10. The method of claim 9, wherein the reaction further comprises a cap analog.

11. The method of claim 10, wherein the cap analog is a dinucleotide cap analog, a trinucleotide cap analog, or a tetranucleotide cap analog.

12. The method of claim 11, wherein the cap analog is a trinucleotide cap analog comprising a GAG sequence.

13. The method of claim 11, wherein the cap analog comprises a compound selected from:

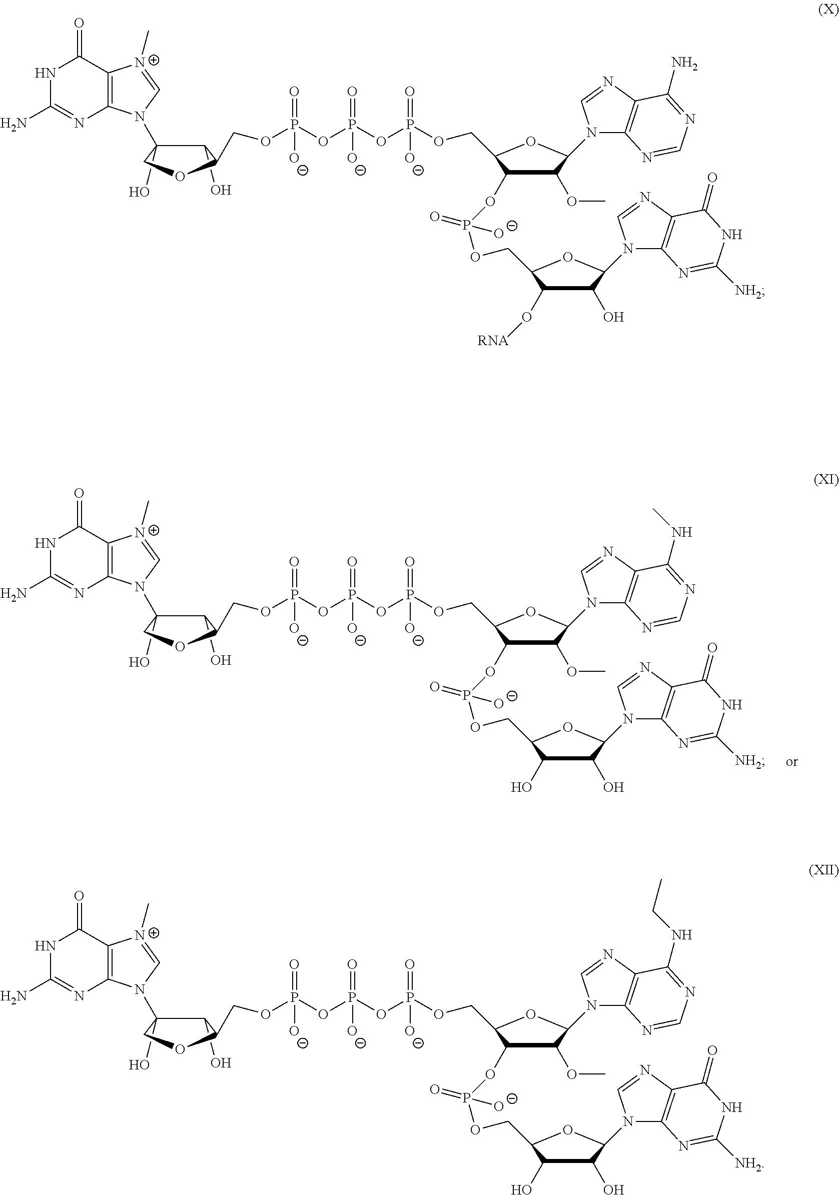

14. The method of claim 11, wherein the tetranucleotide cap analog comprises a GGAG sequence.

15. The method of claim 11, wherein the tetranucleotide cap analog comprises a compound selected from:

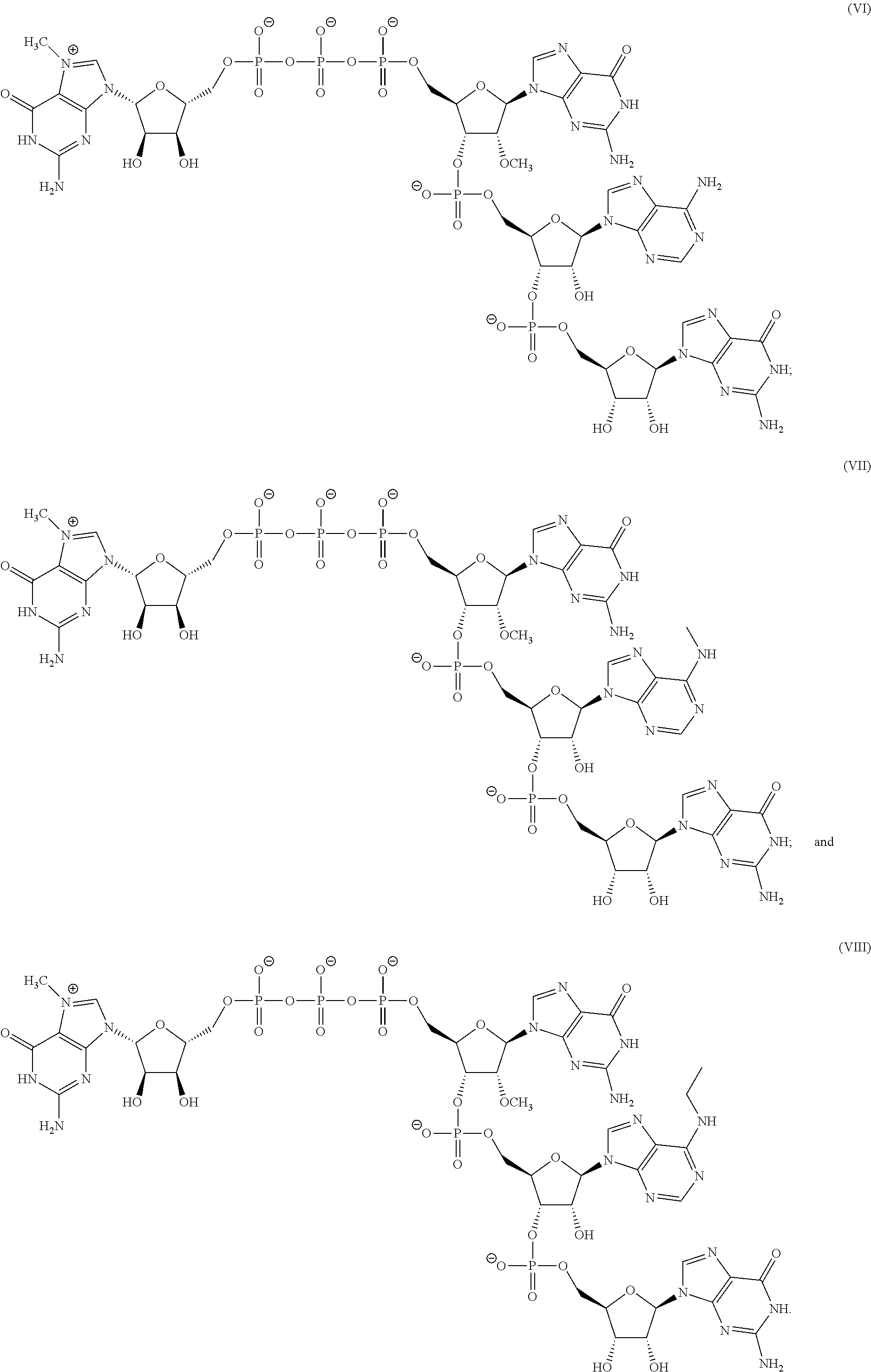

16. The method of claim 9, wherein the DNA includes a 2′-deoxythymidine residue or a 2′-deoxycytidine residue at position +1.

17. A composition or kit comprising the RNA polymerase variant of claim 1 and an in vitro transcription (IVT) reagent selected from the group consisting of a DNA, nucleoside triphosphates, and a cap analog.

18. A nucleic acid encoding the RNA polymerase variant of claim 1.
